SEQUENCE LISTING (48 sequences)

SEQ ID NO: 1 - DNA, 1230 bp, Saccharomyces cerevisiae
  CDS (1)..(1230)
atggttaaagaaactaagttttacgatattctaggtgttccagtaactgccactgatgtcgaaattaagaaagcttatagaaaatgcgccttaaaataccatccagataagaatccaagtgaggaagctgcagaaaagttcaaagaagcttcagcagcctatgaaattttatcagatcctgaaaagagagatatatatgaccaatttggtgaagatggtctaagtggtgctggtggcgctggcggattcccaggtggtggattcggttttggtgacgatatcttttcccaattctttggtgctggtggcgcacaaagaccaagaggtccccaaagaggtaaagatatcaagcatgaaatttctgcctcacttgaagaattatataagggtaggacagctaagttagcccttaacaaacagatcctatgtaaagaatgtgaaggtcgtggtggtaagaaaggcgccgtcaagaagtgtaccagctgtaatggtcaaggtattaaatttgtaacaagacaaatgggtccaatgatccaaagattccaaacagagtgtgatgtctgtcacggtactggtgatatcattgatcctaaggatcgttgtaaatcttgtaacggtaagaaagttgaaaacgaaaggaagatcctagaagtccatgtcgaaccaggtatgaaagatggtcaaagaatcgttttcaaaggtgaagctgaccaagccccagatgtcattccaggtgatgttgtcttcatagtttctgagagaccacacaagagcttcaagagagatggtgatgatttagtatatgaggctgaaattgatctattgactgctatcgctggtggtgaatttgcattggaacatgtttctggtgattggttaaaggtcggtattgttccaggtgaagttattgccccaggtatgcgtaaggtcatcgaaggtaaaggtatgccaattccaaaatacggtggctatggtaatttaatcatcaaatttactatcaagttcccagaaaaccatttcacatcagaagaaaacttgaagaagttagaagaaattttgcctccaagaattgtcccagccattccaaagaaagctactgtggacgaatgtgtactcgcagactttgacccagccaaatacaacagaacacgggcctccaggggtggtgcaaactatgattccgatgaagaagaacaaggtggcgaaggtgttcaatgtgcatctcaatga

SEQ ID NO: 2 - protein, 409 aa, Saccharomyces cerevisiae
MVKETKFYDILGVPVTATDVEIKKAYRKCALKYHPDKNPSEEAAEKFKEASAAYEILSDPEKRDIYDQFGEDGLSGAGGAGGFPGGGFGFGDDIFSQFFGAGGAQRPRGPQRGKDIKHEISASLEELYKGRTAKLALNKQILCKECEGRGGKKGAVKKCTSCNGQGIKFVTRQMGPMIQRFQTECDVCHGTGDIIDPKDRCKSCNGKKVENERKILEVHVEPGMKDGQRIVFKGEADQAPDVIPGDVVFIVSERPHKSFKRDGDDLVYEAEIDLLTAIAGGEFALEHVSGDWLKVGIVPGEVIAPGMRKVIEGKGMPIPKYGGYGNLIIKFTIKFPENHFTSEENLKKLEEILPPRIVPAIPKKATVDECVLADFDPAKYNRTRASRGGANYDSDEEEQGGEGVQCASQ

SEQ ID NO: 3 - DNA, 1263 bp, Triticum aestivum
  CDS (1)..(1263)
atggtaaaagataccaaactatatgatactctgggtatttccccgacctgtactgaagccgagttaaaaaaagcatacaaaatcggagcacttaaacaccatcctgataaaaacgcctcaaatccagccgccgcagaaaaatttaaagaaatatcgcacgcatatgaagtactatctgaccctcaaaaaagacacatatacgaccaatatggcgaagagggccttgagggaggtggtggtgctgcgggagggatgaacgcagaagatttattctctcaattcttcagcggtggctctgccttcggaggtggaggattgggtggcatgttcgggggagggccacagcaacgtggccccccaaaagcccgcaccattcatcacgttcacaaggtatctctagaagatatctaccgcggtaaaatctcaaaactggcactacaaaagtcagtcatatgccacaagtgtgagggacggggtggcaaagatggtgcagtaaaaaaatgtgccggctgtgatggacatggaatgaaaacaatgatgcgtcaaatgggtcctatgattcagcggtttcaaactcactgccccgactgcaatggtgagggagaagtcatccgagagaaagataaatgtaagacgtgtaacggtaaaaagaccaacgtggaacgcaaagtactccacgttcatgtggacagaggtgttcgatcggggcaccggattgaatttaaaggtgaaggagaccaaacccccggagttcaacctggagatgttatctttgaaattgagcagaaaccacatccaagattccaacgaaaagacgatgaccttatttaccacgcagagatcgaccttgttactgccttagcgggcgggtcaatcttcattgagcacttagacgaaagatggctgagtgtggagatacttcctggagaggttatctcacctggatccgttaagatgatacgcggtcagggtatgccatcccatcgtcaccacgactatggaaatatgtttgtacagtttgatgtcaaattccccgaaagtaactttgctgcaaattccgaggcatacgcagctctgaagagtattattccgccgactgtggtacctatcactccacccactgataccatgactgaaactgtatacttcgaagacattgaccctactcaacaagctcgtgcacagggtgcgacagcaatggatgaagacgatgaagatggccatccagccggcgccgaacgggttcaatgtgcgtcacagtaa

SEQ ID NO: 4 - protein, 420 aa, Triticum aestivum
MVKDTKLYDTLGISPTCTEAELKKAYKIGALKHHPDKNASNPAAAEKFKEISHAYEVLSDPQKRHIYDQYGEEGLEGGGGAAGGMNAEDLFSQFFSGGSAFGGGGLGGMFGGGPQQRGPPKARTIHHVHKVSLEDIYRGKISKLALQKSVICHKCEGRGGKDGAVKKCAGCDGHGMKTMMRQMGPMIQRFQTHCPDCNGEGEVIREKDKCKTCNGKKTNVERKVLHVHVDRGVRSGHRIEFKGEGDQTPGVQPGDVIFEIEQKPHPRFQRKDDDLIYHAEIDLVTALAGGSIFIEHLDERWLSVEILPGEVISPGSVKMIRGQGMPSHRHHDYGNMFVQFDVKFPESNFAANSEAYAALKSIIPPTVVPITPPTDTMTETVYFEDIDPTQQARAQGATAMDEDDEDGHPAGAERVQCASQ

SEQ ID NO: 5 - DNA, 1284 bp, Arabidopsis thaliana
  CDS (1)..(1284)
atgcgaagattcaactgggttctgcggcatgtacaagctcgaagaacttttgattccgcgatcggattgcgtcaagggtctcagaagccgttgttcgagcgatacattcacgctacaggtataaacaactccagtgctcgtaattactatgatgttctcggtgtttctcctaaagctacacgggaggagattaaaaaatcatttcatgagcttgcgaaaaaattccaccctgatacaaatagaaataatccgtcagcaaaaaggaagttccaggaaataagagaggcatatgagaccctgggaaattcggaaagaagagaagaatatgataagctgcagtatcggaattcggattatgtaaataatgacggtggtgattcagagaggttcagacgtgcataccagtccaatttctcggatactttccacaagattttttctgagatatttgagaacaatcagataaaacctgatattcgggtggaactgtcgctttctctttccgaagctgcagaagggtgcacaaaacgtttgtcttttgatgcatatgtcttttgtgattcctgtgatgggcttggccaccctagcgatgctgccatgagcatttgtccaacatgcaggggggttggacgagtaactattcctccttttacagcatcatgccagacgtgcaaggggacgggacacattattaaggaatactgcatgtcttgtagaggatcaggtattgtggaaggcacaaagacagctgaacttgtgatccctggaggggtggagtctgaagctacaatcacaatcgtaggtgctggtaatgtaagttcaagaacaagtcaacctgggaacttgtatatcaaactaaaggttgctaatgattcaactttcactagggatggctcagatatatatgtggatgctaatattagctttacacaagctattttggggggcaaagttgtggtgccaacactttcaggcaagatacagctagatataccaaaggggactcagcctgatcaacttcttgttttaagaggcaaaggactaccgaagcaaggcttttttgtagatcatggagatcagtatgttcgcttccgcgttaactttcctactgaagtgaatgaacgtcagcgtgctatactggaagagtttgcaaaggaagaaatcaacaatgagttgagcgactctgctgaaggaagttggtggaatctaacgggtcctcagatcatccgcgacttctcgttaatggtgctgctggcgctattgttgagcaggttaatgggatga

SEQ ID NO: 6 - protein, 427 aa, Arabidopsis thaliana
MRRFNWVLRHVQARRTFDSAIGLRQGSQKPLFERYIHATGINNSSARNYYDVLGVSPKATREEIKKSFHELAKKFHPDTNRNNPSAKRKFQEIREAYETLGNSERREEYDKLQYRNSDYVNNDGGDSERFRRAYQSNFSDTFHKIFSEIFENNQIKPDIRVELSLSLSEAAEGCTKRLSFDAYVFCDSCDGLGHPSDAAMSICPTCRGVGRVTIPPFTASCQTCKGTGHIIKEYCMSCRGSGIVEGTKTAELVIPGGVESEATITIVGAGNVSSRTSQPGNLYIKLKVANDSTFTRDGSDIYVDANISFTQAILGGKVVVPTLSGKIQLDIPKGTQPDQLLVLRGKGLPKQGFFVDHGDQYVRFRVNFPTEVNERQRAILEEFAKEEINNELSDSAEGSWWNLTGPQIIRDFSLMVLLALLLSRLMG

SEQ ID NO: 7 - DNA, 1224 bp, Schizosaccharomyces pombe 972h-
  CDS (1)..(1224)
atggtgaaagaaactaaactatacgaagtcttgaacgttgatgtcactgcttctcaagctgaattgaagaaagcttaccgcaagcttgctttaaaatatcatcccgacaaaaaccctaatgccggcgataagtttaaggaaattagccgtgcttatgaaattcttgctgatgaagagaaacgtgctacttatgatcgttttggtgaagaaggtttacaaggtggtggtgccgatggtggtatgtctgctgatgacttgtttgcttccttttttggtggtggaatgtttggtggtggtatgccccgtggtcctcgcaagggcaaggatcttgttcataccatcaaggttactttggaggacctctatcgtggtaagactacaaagcttgctttgcaaaagaaggtcatttgccccaagtgtagcggtcgtggtggcaaggaaggctctgtcaaatcttgtgcctcttgtaatggtagcggtgtcaaattcattactcgtgccatgggtccaatgatacaacgtatgcaaatgacctgtcctgattgtaatggtgcaggtgaaaccatccgcgatgaagaccgttgcaaagaatgtgatggtgccaaggtcatttctcaacgtaaaatccttaccgtacatgttgagaagggtatgcacaatggtcagaagattgtatttaaggaagaaggcgagcaagctcctggaatcattcccggtgatgtaatctttgtcattgatcaaaaggaacatcctcgtttcaagcgcagtggtgatcatttgttttatgaggctcatgtcgatttactcactgctttggctggtggtcaaattgtcgtcgagcacctggacgaccgttggcttactatccccatcatccccggagagtgcattcgtcccaatgaactcaaagttcttcctggtcaaggtatgctttctcaacgccatcatcaacctggaaacctttacattcgcttccatgtcgactttcctgaacccaacttcgctaccccagaacagcttgcattgcttgaaaaggctttacctccccgtaagattgagagcgctcccaaaaatgctcacactgaggaatgtgttttggcaactgtcgatcctactgagaaggttcgcattgacaataacgtggaccccactactgccacttcgatggatgaagatgaagatgaagaaggtggacaccccggtgttcaatgtgctcaacagtaa

SEQ ID NO: 8 - protein, 407 aa, Schizosaccharomyces pombe 972h-
MVKETKLYEVLNVDVTASQAELKKAYRKLALKYHPDKNPNAGDKFKEISRAYEILADEEKRATYDRFGEEGLQGGGADGGMSADDLFASFFGGGMFGGGMPRGPRKGKDLVHTIKVTLEDLYRGKTTKLALQKKVICPKCSGRGGKEGSVKSCASCNGSGVKFITRAMGPMIQRMQMTCPDCNGAGETIRDEDRCKECDGAKVISQRKILTVHVEKGMHNGQKIVFKEEGEQAPGIIPGDVIFVIDQKEHPRFKRSGDHLFYEAHVDLLTALAGGQIVVEHLDDRWLTIPIIPGECIRPNELKVLPGQGMLSQRHHQPGNLYIRFHVDFPEPNFATPEQLALLEKALPPRKIESAPKNAHTEECVLATVDPTEKVRIDNNVDPTTATSMDEDEDEEGGHPGVQCAQQ

SEQ ID NO: 9 - DNA, 1254 bp, Gibberella zeae PH-1
  CDS (1)..(1254)
atggtcaaggaaaccaagtactacgacacactgggtgtcgcccctactgctactgagcaggaactgaagaaggcttacaaggtcggagccctcaagtaccaccctgacaagaacgcacacaaccccgatgccgaagaaaagttcaaggaggtttcgcatgcctacgaaattctctccgatccccagaagcgacaagtctacgaccagtatggtgaggccggcctcgagggcggtgccggaggtggtggcatggccgctgaggacttgtttgctcagttcttcggaggtggtggcttcggtggcatgggcggtatgtttggcggcggcggtatgaaccgcggcccccccaaggcccgaaccattcaccacacccacaaggtatcactagaagacatctatcggggtaagatctcgaagcttgctcttcaacggtcaatcatttgccctaagtgcgaaggccttggtggaaaggagggtgcggttaagcgttgcactggctgtgatggccacggtatgaagaccatgatgcgccagatgggtcccatgatccagcgcttccagactgtctgccccgactgtaacggagagggtgagatcatcaaggagaaggaccgctgcaagcagtgcaacggaaagaagaccactgtcgaccgcaaggtcctccacgtccacgtcgacaagggtgtccgcagtggcaccaaggtcgagttccgaggcgagggtgaccaagcaccaggtgttcaggcaggtgacgtcgttttcgagattgagcagaagccccatgctcgcttcactcgtcgcgaagatgatttgctttataactgcgatattgagcttgttacagctttggctggtggtaccatctacatcgagcacctcgatgaccgatggctggccgttgacatcctccccggcgaggctatctctcaagatgctgtcaagatgattcgcggccagggtatgccttcgcccaggcaccacgacttcggcaacatgtaccttaagttcaacgtcaagttccccgagaagaactggaccgatgacgccgagactttcgagactctccgaaaggttctccccgctccctccgtccagaacatcccccccggtgatgctatgtctgagcccgccagcctcgaggatctcgacaactccgcccaaagcagagtcttcggtggctctgatggcatgatggatgacgatgacgaggatggccaccccggtggtgagcgcgtgcagtgcgcttcccagtaa

SEQ ID NO: 10 - protein, 417 aa, Gibberella zeae PH-1
MVKETKYYDTLGVAPTATEQELKKAYKVGALKYHPDKNAHNPDAEEKFKEVSHAYEILSDPQKRQVYDQYGEAGLEGGAGGGGMAAEDLFAQFFGGGGFGGMGGMFGGGGMNRGPPKARTIHHTHKVSLEDIYRGKISKLALQRSIICPKCEGLGGKEGAVKRCTGCDGHGMKTMMRQMGPMIQRFQTVCPDCNGEGEIIKEKDRCKQCNGKKTTVDRKVLHVHVDKGVRSGTKVEFRGEGDQAPGVQAGDVVFEIEQKPHARFTRREDDLLYNCDIELVTALAGGTIYIEHLDDRWLAVDILPGEAISQDAVKMIRGQGMPSPRHHDFGNMYLKFNVKFPEKNWTDDAETFETLRKVLPAPSVQNIPPGDAMSEPASLEDLDNSAQSRVFGGSDGMMDDDDEDGHPGGERVQCASQ

SEQ ID NO: 11 - DNA, 1224 bp, Candida glabrata CBS 138
  CDS (1)..(1224)
atggttaaggatactaagttgtacgacacgctgggtgtgtcgcctggtgcgagcgatgcagagatcaagaaggcgtacaggaagagtgcgttgaagtaccatccggacaagaacccttctgaggaggctgctgagaagtttaaggaggtttccagtgcttatgagattctttcagactcgcagaagcgtgaggtgtacgaccagttcggtgaggaaggtctgagcggaaacggtggtgccggtttcccaggcggcttcggttttggcgaggacatcttctcgcagttcttcggcggtgccaccggtggcaggcctagaggtcctcagcgtggtagggacatcaagcacgagatggctgcctctctggaggagctttacaagggtagaaccgccaagctggcgctgaacaagcagatcttgtgtaagagctgtgaaggtagaggtggtaaggaaggcgctgtcaagaagtgtagcagctgtaacggtcaaggtatcaagttcgtcaccagacagatgggtcctatgatccagagattccaaaccgagtgtgacgtgtgtcacggtacaggtgatatcatagacgccaaggaccgttgtaagtcttgtaacggtaagaaggtcgacaacgagagaaagatccttgaggtccgtatcgagccaggtatgaaagacggccagaagatcgtcttcaaaggtgaagccgaccaagcaccagatgtcatccctggtgatgtcgtcttcgtgatcagtgaaaagccacacaagcacttccaaagagccggtgacgacttgatctacgaggccgagatcgacctactaaccgctttggccggtggccagttcgccctggaacacgtttccggtgactggttgaaggtcgatatcgttccaggtgaagttatcgccccaggtgcccgcaagatcgtcgaaggtaaaggtatgcctattcagaaatacggtggttacggtaacttgttgatcaagttcaacatcaagttcccagaaaaccacttcacatccgaagaaaacttgaagaaactggaagaaatcttgccaccaagaagacaaatcaacatacctgccaaggcccaagtcgatgactgtgttctaagcgaattcgacccatccaagtttggtcaatccaatggtagaagcggtgcaaactacgactctgacgacgaggatgcccacggtggtgaaggtgttcaatgtgcatctcaatga

SEQ ID NO: 12 - protein, 407 aa, Candida glabrata CBS 138
MVKDTKLYDTLGVSPGASDAEIKKAYRKSALKYHPDKNPSEEAAEKFKEVSSAYEILSDSQKREVYDQFGEEGLSGNGGAGFPGGFGFGEDIFSQFFGGATGGRPRGPQRGRDIKHEMAASLEELYKGRTAKLALNKQILCKSCEGRGGKEGAVKKCSSCNGQGIKFVTRQMGPMIQRFQTECDVCHGTGDIIDAKDRCKSCNGKKVDNERKILEVRIEPGMKDGQKIVFKGEADQAPDVIPGDVVFVISEKPHKHFQRAGDDLIYEAEIDLLTALAGGQFALEHVSGDWLKVDIVPGEVIAPGARKIVEGKGMPIQKYGGYGNLLIKFNIKFPENHFTSEENLKKLEEILPPRRQINIPAKAQVDDCVLSEFDPSKFGQSNGRSGANYDSDDEDAHGGEGVQCASQ

SEQ ID NO: 13 - DNA, 1230 bp, Kluyveromyces lactis NRRL Y-1140
  CDS (1)..(1230)
atggttaaggatacaaaactatacgatcttttgggggtttctccaggtgctgatgacaaccaaatcaagaaggcttatagaaaaagcgccttaaaattccatccagataagaatccaagtgaagaagctgctgagaaattcaaagaaatcacttccgcttacgaaatcttatctgattctcaaaagagagaagtatatgaccagtttggtttggaaggtctttctggccaaggtgcaggcggtccaggtggcttcggtggcttcggtgaagacttattctctcaattctttggtggcggtagttcaagaccaagaggtcctcaaaagggtagggatattagacatgaaattccagccactttagaacaattattcaaaggtagaactgccaaactggcattaaacaaacagttaatctgtaagtcatgtgaaggtcgtggtggtaaggaaggtagtgttaagaaatgtactgcttgtagcggtcaaggtttcaagttcgtaaccagacaaatgggtcctatgatccaaagattccaagttgagtgtgaatcttgtcatggtgctggtgaaataatcgatccaaagggccgttgtaaggtctgtagcggtaagaaggttgtaaacgaaagaaaggttttagaagtcaacattgaaccaggtatgaaagacggccaaagaatcgttttccagggtgaagctgaccaatctccaggtattattccaggtgacgttgtctttgtcgtttctgaacaaccacatccagtcttcaaaagagatggtaacgacttacactacgacgctgaaatcgatttgctatctgctattgcaggtggtcaatttgccgtcaaacacgtatcaggtgaatatttgaaggtcgaaatcgtaccaggtgaagtgatctctccaggttctgttaaagttatagaaggcaaaggtatgccaattccaaaatacggtggttacggtaacttgttgatcaagttcaacatcaagttcccacctgcacacttcacggacgatgaaaccttgaagaagctcgaggaaattctaccaccaagaaatgtaccttctattcctgcggacgcagaagttgaagattgtgttttagcagacttcgactccagtaagcatggtgctagagctggtggtaacggcagaggccaaagctatgattcagatgatgaagacggacaccacggtgctgaaggtgttcaatgtgcctcacagtaa

SEQ ID NO: 14 - protein, 409 aa, Kluyveromyces lactis NRRL Y-1140
MVKDTKLYDLLGVSPGADDNQIKKAYRKSALKFHPDKNPSEEAAEKFKEITSAYEILSDSQKREVYDQFGLEGLSGQGAGGPGGFGGFGEDLFSQFFGGGSSRPRGPQKGRDIRHEIPATLEQLFKGRTAKLALNKQLICKSCEGRGGKEGSVKKCTACSGQGFKFVTRQMGPMIQRFQVECESCHGAGEIIDPKGRCKVCSGKKVVNERKVLEVNIEPGMKDGQRIVFQGEADQSPGIIPGDVVFVVSEQPHPVFKRDGNDLHYDAEIDLLSAIAGGQFAVKHVSGEYLKVEIVPGEVISPGSVKVIEGKGMPIPKYGGYGNLLIKFNIKFPPAHFTDDETLKKLEEILPPRNVPSIPADAEVEDCVLADFDSSKHGARAGGNGRGQSYDSDDEDGHHGAEGVQCASQ

SEQ ID NO: 15 - DNA, 1221 bp, Debaryomyces hansenii CBS767
  CDS (1)..(1221)
atggttaaggaaacaaagttttacgatcaattaggtgtgtcgccatcagctggagataccgaattaaagaaagcttatagaaaggctgcattgaaatatcatccagataagaatccatcaccagaagccgctgaaaagttcaaggaactctcacatgcttacgagattctttcggatgaacagaagagagaagtgtatgatagctatggtgaagaagggttatcaggtgccggtggtatgggcggcggtatgggagcagaagacatcttttcgcaattcttcggaggaggatttggcggtatgggtggcggagcttcccgtggaccagctagaggcaaggacatcaagcactcgatcagttgtaccttagaagagttgtacaagggtagaactgcgaaattggcattgaacaagactatattgtgtaagacatgtgaaggtcgtggtggtaaagaaggtaagatcaagcagtgttcttcttgtcacggtcaaggtatgaagtttgtcacaagacaaatgggtcctatgatacaaagattccaaaccgtttgtgacgcttgtcaaggttctggtgacatctgtgacgctaaggaccgttgtaccgcgtgtaagggtaagaagactcaaactgaacgtaagatattacaagtccacattgatcctggtatgaaggatggccaaagaatcgtattcagcggtgaaggtgaccaagaaccaggtgtcactcctggtgatgtcgtgtttgttgttgacgaaaagcaacacgaaaagttcaccagaaaggctaacgacttgtactacgaagctgaagtggatttagccactgccttgactggtggtgaacttgccttcaagcacgtttctggcgactacatcaagatcccaatcaccccaggtgaagttattgccccaggtgttaccaaggtaattgaaaaccaaggtatgccaatctacagacacggtggcaacggtcatatgtttgttaaattcaccgttaagttcccaaagaacaactttgctactgaagcaaaattgaaggaattagaagctattttacctccaaaggctaaggttaccattccaaagggtactgaggttgatgaatgtgaattggtcgatgttgacccacgtaagcatcaatctgctggaagacgtgatgcttatgattctgatgacgaagaaggtggtgccggcccaggtgtccaatgtgcatctcaatag

SEQ ID NO: 16 - protein, 406 aa, Debaryomyces hansenii CBS767
MVKETKFYDQLGVSPSAGDTELKKAYRKAALKYHPDKNPSPEAAEKFKELSHAYEILSDEQKREVYDSYGEEGLSGAGGMGGGMGAEDIFSQFFGGGFGGMGGGASRGPARGKDIKHSISCTLEELYKGRTAKLALNKTILCKTCEGRGGKEGKIKQCSSCHGQGMKFVTRQMGPMIQRFQTVCDACQGSGDICDAKDRCTACKGKKTQTERKILQVHIDPGMKDGQRIVFSGEGDQEPGVTPGDVVFVVDEKQHEKFTRKANDLYYEAEVDLATALTGGELAFKHVSGDYIKIPITPGEVIAPGVTKVIENQGMPIYRHGGNGHMFVKFTVKFPKNNFATEAKLKELEAILPPKAKVTIPKGTEVDECELVDVDPRKHQSAGRRDAYDSDDEEGGAGPGVQCASQ

SEQ ID NO: 17 - DNA, 1254 bp, Yarrowia lipolytica CLIB122
  CDS (1)..(1254)
atggtcaaggaatcaaaactctacgacgttctcggcgtcagcgtaaccgccaccgaagtggaaatcaaaaaggcataccgagttggtgctctcaaataccaccccgataagaaccccggcaacgtcgaggccgaggccaagttcaaggaaatctccatggcctacgaggttctgtcaaacgaccagaagagagctgcatacgacaactttggagaggctggcctcggcggaggagccgacggaggaatgggcggcggatctgctgaggagctgttttcgcatttctttggcggcggcggtggcatgggcggcatgggcggcatgttcggcggcggccagccccagggcccccgacgatcccgtgacattgttcacgccgtgtccgtgaccctcgaggacctgttccgaggaaagacctccaagatggctctcaagaagaccgtgctctgtaacggctgtgacggtatcggaggcaaggccggctccgtcaacaagtgtgagacctgtaagggtcagggattcaagtttgtcacccgacaaatgggccccatgctgcagcgataccagaccaagtgtaacgactgtaacggcgagggcgagatcatcgaccccaaggatcgatgcaaggactgtaacggaagaaagaccaaggaggagcgaaaggtgctcgaggtcaacattgataagggtatggtcaacggacagaagatcaccttctctggtgagggtgaccagggtcctgatattattcctggtgacgttgtctttgtgctggatgagcagccccacgcccgatttgtccgacgaggcgacgatctgtactaccacgccaagattgatctcaacactgcccttaccggtggctctttcatgattgagcatcttgagaaggaggagtggatcaaggtggagatcatccccggcgagatcatttcgcatggcaccaccaaggtcgtggagggcaagggtatgccctcctaccgacaccaggttcacggcaacctgttcattcagtttgaggtcgagttccccgcatctggctctctcaacgaggagactctgcaacagctgtctgctcttctgcctgccaagcctgctcttcccagtgtgcccgagagtgtccatgttgacgatgttgttctggctgacgtggatcctctcaagcaccgaggagctatgggcggcgatgatgagatggacatggatgaggatggacctggaggagcccagggtgtccagtgtgcttctcagtaa

SEQ ID NO: 18 - protein, 417 aa, Yarrowia lipolytica CLIB122
MVKESKLYDVLGVSVTATEVEIKKAYRVGALKYHPDKNPGNVEAEAKFKEISMAYEVLSNDQKRAAYDNFGEAGLGGGADGGMGGGSAEELFSHFFGGGGGMGGMGGMFGGGQPQGPRRSRDIVHAVSVTLEDLFRGKTSKMALKKTVLCNGCDGIGGKAGSVNKCETCKGQGFKFVTRQMGPMLQRYQTKCNDCNGEGEIIDPKDRCKDCNGRKTKEERKVLEVNIDKGMVNGQKITFSGEGDQGPDIIPGDVVFVLDEQPHARFVRRGDDLYYHAKIDLNTALTGGSFMIEHLEKEEWIKVEIIPGEIISHGTTKVVEGKGMPSYRHQVHGNLFIQFEVEFPASGSLNEETLQQLSALLPAKPALPSVPESVHVDDVVLADVDPLKHRGAMGGDDEMDMDEDGPGGAQGVQCASQ

SEQ ID NO: 19 - DNA, 1017 bp, Candida albicans SC5314
  CDS (1)..(1017): transl_table=12
atggttaaagacacaaagttttacgatgccttgggggtatcaccaaatgcatctgatgctgaattgaaaaaagcttatagaaaagctgctttaaaatatcatccagataaaaatccttccccagaagcagctgaaaagtttaaagaactttctcatgcttatgaaattttgagtgatgaccaaaaaagagaaatatatgatcaatatggtgaagaagggttgagtggacaaggtgctggaggcttcggtatgaatgccgatgacatctttgcccaattctttggtggtggtttccatggaggtccacaacgtccatctagaggtaaagacatcaagcattccattgcttgttccttagaagagttatacaagggtaaaactgtcaagttggccttgaacaaaactgtcttgtgtggcgaatgtaagggccgtggaggagcagaaggtaaggttgcacaatgtcctgattgtcacggtaatggtatgaaatttgttactaagcaaatgggtccaatgattcaaagattccaaactgtatgtgacaagtgtcaaggtactggtgatttgattgatccaaaggatcgttgtaagaaatgtaacggtaaaaaaaccgagtcggaaagaaagattttggaagttcatgtgaaacctggtatgaaggatggagatcatattacatttgctggagaaggtgatcaaacacctggagtaacacctggtgatgttgtattcattatatctcagaaaccacacccagtgttccaaagaaaaggtaatgatttattgattgaacaagagattgaattggctacagcattggctggtggtgaaattgctttcaaacacatttcaggtgattgggttagaattgaaattccagctggtgaagttattgctccaggatctattaaaatggttgaaggatttggtatgccagtcagaactcacaaaggtaatttaataatccatttcaatgtcaaattcccagaaaataattttgctgatgaagaagcttga

SEQ ID NO: 20 - protein, 338 aa, Candida albicans SC5314
MVKDTKFYDALGVSPNASDAELKKAYRKAALKYHPDKNPSPEAAEKFKELSHAYEILSDDQKREIYDQYGEEGLSGQGAGGFGMNADDIFAQFFGGGFHGGPQRPSRGKDIKHSIACSLEELYKGKTVKLALNKTVLCGECKGRGGAEGKVAQCPDCHGNGMKFVTKQMGPMIQRFQTVCDKCQGTGDLIDPKDRCKKCNGKKTESERKILEVHVKPGMKDGDHITFAGEGDQTPGVTPGDVVFIISQKPHPVFQRKGNDLLIEQEIELATALAGGEIAFKHISGDWVRIEIPAGEVIAPGSIKMVEGFGMPVRTHKGNLIIHFNVKFPENNFADEEA

SEQ ID NO: 21 - DNA, 1182 bp, Candida albicans SC5314
  CDS (1)..(1182): transl_table=12
atggttaaagacacaaagttttacgatgccttgggggtatcaccaaatgcatctgatgctgaattgaaaaaagcttatagaaaagctgctttaaaatatcatccagataaaaatccttccccagaagcagctgaaaagtttaaagaactttctcatgcttatgaaattttgagtgatgaccaaaaaagagaaatatatgatcaatatggtgaagaagggttgagtggacaaggtgctggaggcttcggtatgaatgccgatgacatctttgcccaattctttggtggtggtttccatggaggtccacaacgtccatctagaggtaaagacatcaagcattccattgcttgttccttagaagagttatacaagggtaaaactgtcaagttggccttgaacaaaactgtcttgtgtggcgaatgtaagggccgtggaggagcagaaggtaaggttgcacaatgtcctgattgtcacggtaatggtatgaaatttgttactaagcaaatgggtccaatgattcaaagattccaaactgtatgtgacaagtgtcaaggtactggtgatttgattgatccaaaggatcgttgtaagaaatgtaacggtaaaaaaaccgagtcggaaagaaagattttggaagttcatgtgaaacctggtatgaaggatggagatcatattacatttgctggagaaggtgatcaaacacctggagtaacacctggtgatgttgtattcattatatctcagaaaccacacccagtgttccaaagaaaaggtaatgatttattgattgaacaagagattgaattggctacagcattggctggtggtgaaattgctttcaaacacatttcaggtgattgggttagaattgaaattccagctggtgaagttattgctccaggatctattaaaatggttgaaggatttggtatgccagtcagaactcacaaaggtaatttaataatccatttcaatgtcaaattcccagaaaataattttgctgatgaagaaagcttgaagaaattggctagtcttttaccaaaaccaaaagaagtcaagatcccagccgatgccgatgttgatgactgtaccatggtcccagctaaattggaacaaagtaatccatacgagtcagatgaagaagctcacggaggtccaggggtccaatgtgctagtcaatag

SEQ ID NO: 22 - protein, 393 aa, Candida albicans SC5314
MVKDTKFYDALGVSPNASDAELKKAYRKAALKYHPDKNPSPEAAEKFKELSHAYEILSDDQKREIYDQYGEEGLSGQGAGGFGMNADDIFAQFFGGGFHGGPQRPSRGKDIKHSIACSLEELYKGKTVKLALNKTVLCGECKGRGGAEGKVAQCPDCHGNGMKFVTKQMGPMIQRFQTVCDKCQGTGDLIDPKDRCKKCNGKKTESERKILEVHVKPGMKDGDHITFAGEGDQTPGVTPGDVVFIISQKPHPVFQRKGNDLLIEQEIELATALAGGEIAFKHISGDWVRIEIPAGEVIAPGSIKMVEGFGMPVRTHKGNLIIHFNVKFPENNFADEESLKKLASLLPKPKEVKIPADADVDDCTMVPAKLEQSNPYESDEEAHGGPGVQCASQ

SEQ ID NO: 23 - DNA, 1242 bp, Aspergillus niger
  CDS (1)..(1242)
atggtcaaggaaactaagttctacgacatcctgggggttcccccgacggcctctgaggcccaactcaagactgcctacaagaagggtgccctgaagtaccaccctgacaagaacacaaacaaccccgaagccgctgagaagttcaaggaattgtctgccgcttacgagaccctctccgatccccagaagcgtagcctctacgaccagctcggtgaggagggtcttgagcatggcggtgctggcggtggcatgggcgccgaggacctctttgctcagttcttcggcggcggcggtggtttcggtggcatgttcggtggtggcatgcgtgaccagggccccaagaaggctcgtaccatccaccacgttcacaaggtcaacctcgaggatatctaccgcggtaaggtctcgaagctggctctgcagaagtcagtcatttgcccgggttgtgatggtcgcggtggtaaggagggtgctgtcaagtcgtgtaccggctgcaatggttccggtatgaagaccatgatgcgccagatgggtcccatgatccagcgcttccagacggtttgccccgactgcaatggtgagggcgagatcatccgtgagaaggaccgctgcaagcgctgtaacggcaagaagaccaccgtggagcgcaaggttctccacgtccacgttgaccgtggtgtgaagaacggccacaagattgaattccgcggcgagggtgaccagatgcctggtgttctgcccggcgacgttgttttcgagattgagcagaagccccacccccgcttccagcgcaaggacgacgacctcttctaccaggccgagatcgacctgctcactgctcttggcggtggtaccatcaacattgagcaccttgatgaccggtggttgaccgtgaccgtcgctccgggcgaggtcatcactcctggtgctatcaaggttatcaagggtcagggtatgccttcctaccgccaccacgacttcggcaacctctacatccaattcgatgtcaagttccccgagaaggatcagctcaagaacctcgagttgctcgagcaggtcctgcctcctcgtatggagcagtcccagcccccgcaggacgccatgatcgaggactttgagctggaggacattgatggcagcgagtcgtctcaggctcgcgcacacggtgccgccagcgccatggacgaggatgacgaggatgttcctcctggcgctgagcgtgtgcagtgcgcatcgcagtaa

SEQ ID NO: 24 - protein, 413 aa, Aspergillus niger
MVKETKFYDILGVPPTASEAQLKTAYKKGALKYHPDKNTNNPEAAEKFKELSAAYETLSDPQKRSLYDQLGEEGLEHGGAGGGMGAEDLFAQFFGGGGGFGGMFGGGMRDQGPKKARTIHHVHKVNLEDIYRGKVSKLALQKSVICPGCDGRGGKEGAVKSCTGCNGSGMKTMMRQMGPMIQRFQTVCPDCNGEGEIIREKDRCKRCNGKKTTVERKVLHVHVDRGVKNGHKIEFRGEGDQMPGVLPGDVVFEIEQKPHPRFQRKDDDLFYQAEIDLLTALGGGTINIEHLDDRWLTVTVAPGEVITPGAIKVIKGQGMPSYRHHDFGNLYIQFDVKFPEKDQLKNLELLEQVLPPRMEQSQPPQDAMIEDFELEDIDGSESSQARAHGAASAMDEDDEDVPPGAERVQCASQ

SEQ ID NO: 25 - DNA, 1242 bp, Aspergillus oryzae
  CDS (1)..(1242)
atggtcaaggaaaccaagttttacgacgttctcggggttgcccccacagccacagaggctcaactgaagaccgcctataagaagggtgccctcaaatatcaccctgacaagaacgcaaacaaccccgatgctgctgaaaagttcaaggagctctcccgtgcctatgaaattctctccgactcccagaagcgttctatttacgaccagctcggtgaggagggtctcgaaaatggtggaggcgccggtggaatgggtgctgaggatctctttgcccagttcttcggtggcggcggtggctttggaggtatgtttggtggtggcatgcgggagcagggccccaagaaggcccgcaccatccatcacgttcacaaggtcaacctggaggacatctaccgtggaaaggtttcgaagttggccctgcagaagtctgtcatttgccctggctgtgatggccgtggtggtaaggaaggtgccgtcaagtcgtgtggcggctgcaatggtaccggtatgaagactatgatgcgccagatgggacctatgatccagcggttccagactgtttgcccagactgcagtggtgagggtgagaccattcgggagcgcgatcgctgcaagcgctgcaacggtaagaagaccgttgttgagcgcaaggtcctccacgtccatgtcgacaagggtgtcaggaacggccacaagatcgagttccgtggggagggtgaccagatgcctggcgtcctacccggagatgtggtcttcgagattgaacagaagcctcacccccggttccagcgtaaggaagatgacctcttctatcacgctgaaatcgatcttctcacagctcttgctggcggtaccatcaacattgagcacctcgatgaccgctggttgactgtgaacatcgcacctggcgaggttgttactcctggcgctatcaaggtgatcaagggccagggtatgccgtcattccgccaccatgacttcggcaacctctatattcagtttgacgtcaagttccccgagaaggatcagctcaacaacctcaaccttttggaacaggttctgcccccccggatggagcagcctcaaccacctaccgattctatggtggaggacttcgagctggaggacattgactctagcgagtactcccaggcacgcgcccatggtgctgccggttccatggatgaggatgatgacgacgttcctcctggtgctgagagagtgcagtgcgcctctcagtaa

SEQ ID NO: 26 - protein, 413 aa, Aspergillus oryzae
MVKETKFYDVLGVAPTATEAQLKTAYKKGALKYHPDKNANNPDAAEKFKELSRAYEILSDSQKRSIYDQLGEEGLENGGGAGGMGAEDLFAQFFGGGGGFGGMFGGGMREQGPKKARTIHHVHKVNLEDIYRGKVSKLALQKSVICPGCDGRGGKEGAVKSCGGCNGTGMKTMMRQMGPMIQRFQTVCPDCSGEGETIRERDRCKRCNGKKTVVERKVLHVHVDKGVRNGHKIEFRGEGDQMPGVLPGDVVFEIEQKPHPRFQRKEDDLFYHAEIDLLTALAGGTINIEHLDDRWLTVNIAPGEVVTPGAIKVIKGQGMPSFRHHDFGNLYIQFDVKFPEKDQLNNLNLLEQVLPPRMEQPQPPTDSMVEDFELEDIDSSEYSQARAHGAAGSMDEDDDDVPPGAERVQCASQ

SEQ ID NO: 27 - DNA, 1329 bp, Arabidopsis thaliana
  CDS (1)..(1329)
atggctataatacaacttggaagtacatgtgttgctcaatggagtattcgtcctcaatttgcagtcagagcttattatcccagcagaatcgaatcaactcgccatcaaaattccagtagccaagtaaattgtttgggagcttcaaagtcgagtatgttctcacatgggtcattgcccttcttgtccatgacgggaatgtccagaaatatgcatcctcctcgcagaggatctcgcttcactgttagagctgatgcagattactattcggtactcggagtttcgaaaaatgcaaccaaagctgagattaaaagcgcttatcggaagctggctaggaattaccatccggatgtgaacaaggatcctggcgcagaagagaaattcaaagaaataagtaacgcatatgaggttttatcagatgatgaaaagaaatctctttacgataggtatggtgaggccggacttaaaggcgctgctggatttggcaatggggattttagtaatccgttcgatctattcgactcattattcgaaggcttcggtggtgggatgggtagaggttcaagaagcagagctgtggatggtcaagacgagtattacacgctaatcttaaacttcaaagaagcggttttcggaatggagaaagaaatagagatatcccggctcgagagctgtgggacatgtgaaggttcaggtgcaaaacctggaaccaaaccgaccaaatgcaccacgtgtggcggacaaggccaagtggtttcagcagctagaactcctctgggcgtgttccaacaagtcatgacttgctcgtcctgtaatggcactggagagatctcgacgccgtgtggtacttgctctggagacggacgcgtgaggaagacaaaacggataagtctcaaagtaccagccggggttgattcaggtagccggttgagagtgagaggagaaggcaatgccgggaagagaggcggatcaccgggcgatctgtttgttgttatagaagttatcccagacccgattttgaaacgagacgatacaaatattctctacacttgcaagatatcgtatatcgatgcgattttagggacgacactgaaagtaccaacagtggatgggacagtagatttgaaagttccagctgggacacaacctagcacgacgcttgtgatggcgaaaaaaggagttccggtattgaacaagagtaatatgagaggagatcagttggtgagagtacaagtggagatacctaagagattgagcaaagaggagaaaaaacttattgaagagcttgctgatatgagcaagaacaggactgctaatagcaccagtagatga

SEQ ID NO: 28 - protein, 442 aa, Arabidopsis thaliana
MAIIQLGSTCVAQWSIRPQFAVRAYYPSRIESTRHQNSSSQVNCLGASKSSMFSHGSLPFLSMTGMSRNMHPPRRGSRFTVRADADYYSVLGVSKNATKAEIKSAYRKLARNYHPDVNKDPGAEEKFKEISNAYEVLSDDEKKSLYDRYGEAGLKGAAGFGNGDFSNPFDLFDSLFEGFGGGMGRGSRSRAVDGQDEYYTLILNFKEAVFGMEKEIEISRLESCGTCEGSGAKPGTKPTKCTTCGGQGQVVSAARTPLGVFQQVMTCSSCNGTGEISTPCGTCSGDGRVRKTKRISLKVPAGVDSGSRLRVRGEGNAGKRGGSPGDLFVVIEVIPDPILKRDDTNILYTCKISYIDAILGTTLKVPTVDGTVDLKVPAGTQPSTTLVMAKKGVPVLNKSNMRGDQLVRVQVEIPKRLSKEEKKLIEELADMSKNRTANSTSR

SEQ ID NO: 29 - DNA, 1329 bp, Arabidopsis thaliana
  CDS (1)..(1329)
atggctataatacaacttggaagtacatgtgttgctcaatggagtattcgtcctcaatttgcagtcagagcttattatcccagcagaatcgaatcaactcgccatcaaaattccagtagccaagtaaattgtttgggagcttcaaagtcgagtatgttctcacatgggtcattgcccttcttgtccatgacgggaatgtccagaaatatgcatcctcctcgcagaggatctcgcttcactgttagagctgatgcagattactattcggtactcggagtttcgaaaaatgcaaccaaagctgagattaaaagcgcttatcggaagctggctaggaattaccatccggatgtgaacaaggatcctggcgcagaagagaaattcaaagaaataagtaacgcatatgaggttttatcagatgatgaaaagaaatctctttacgataggtatggtgaggccggacttaaaggcgctgctggatttggcaatggggattttagtaatccgttcgatctattcgactcattattcgaaggcttcggtggtgggatgggtagaggttcaagaagcagagctgtggatggtcaagacgagtattacacgctaatcttaaacttcaaagaagcggttttcggaatggagaaagaaatagagatatcccggctcgagagctgtgggacatgtgaaggttcaggtgcaaaacctggaaccaaaccgaccaaatgcaccacgtgtggcggacaaggccaagtggtttcagcagctagaactcctctgggcgtgttccaacaagtcatgacttgctcgtcctgtaatggcactggagagatctcgacgccgtgtggtacttgctctggagacggacgcgtgaggaagacaaaacggataagtctcaaagtaccagccggggttgattcaggtatccggttgagagtgagaggagaaggcaatgccgggaagagaggcggatcaccaggcgatctgtttgttgttatagaagttatcccagacccgattttgaaacgagacgatacaaatgttctctacacttgcaagatatcgtatatcgatgcgattttagggacgacactgaaagtaccaacagtggatgggacagtagatttgaaagttccagctgggacacaacctagcacgacgcttgtgatggccaaaaaaggagttccggtattgaacaagagtaatatgagaggagatcagttggtgagagtacaagtggagatacctaagagattgagcaaagaggagaaaaaacttattgaagagcttgctgatatgagcaagaacaagactgctaatagcaccagtagatga

SEQ ID NO: 30 - protein, 442 aa, Arabidopsis thaliana
MAIIQLGSTCVAQWSIRPQFAVRAYYPSRIESTRHQNSSSQVNCLGASKSSMFSHGSLPFLSMTGMSRNMHPPRRGSRFTVRADADYYSVLGVSKNATKAEIKSAYRKLARNYHPDVNKDPGAEEKFKEISNAYEVLSDDEKKSLYDRYGEAGLKGAAGFGNGDFSNPFDLFDSLFEGFGGGMGRGSRSRAVDGQDEYYTLILNFKEAVFGMEKEIEISRLESCGTCEGSGAKPGTKPTKCTTCGGQGQVVSAARTPLGVFQQVMTCSSCNGTGEISTPCGTCSGDGRVRKTKRISLKVPAGVDSGIRLRVRGEGNAGKRGGSPGDLFVVIEVIPDPILKRDDTNVLYTCKISYIDAILGTTLKVPTVDGTVDLKVPAGTQPSTTLVMAKKGVPVLNKSNMRGDQLVRVQVEIPKRLSKEEKKLIEELADMSKNKTANSTSR

SEQ ID NO: 31 - DNA, 1386 bp, Arabidopsis thaliana
  CDS (1)..(1386)
atggtcccttccaatggcgcaaaggttcttcgcttgttgagtcgtcgatgtctctcttcatcgcttattcaagatttagccaatcagaaactgagaggagtatgtattgggagttataggagattgaatacgagtgttggcaatcatgctaacgtgattggagattacgcttcaaaatctggacatgatcggaaatggatcaactttggaggttttaatactaattttggttctacaaggtcttttcatggaacaggttcttcgtttatgtctgctaaggactactatagtgttcttggagtgagtaagaatgctcaagaaggtgaaatcaagaaggcttattatgggcttgctaagaaactccatcctgatatgaataaagatgatccagaagctgagacgaagttccaggaagtctcaaaagcatatgaaattttgaaagataaggagaagcgtgacctttatgaccaggttggtcatgaagcatttgagcaaaatgctagtggtggatttccaaatgatcaaggcttcggtggtggtggtggtggtgggtttaacccatttgatatctttgggagcttcaatggcgatatttttaacatgtaccggcaagatattggaggtcaagatgtcaaggttttgcttgatctttctttcatggaagctgttcaaggatgctccaaaactgtgacttttcaaaccgagatggcttgtaatacttgtggtggacaaggtgttcctcctggtaccaaacgagagaaatgcaaagcctgtaatggctctgggatgtttcatttttcagacctcactgaggaggggtatgttaagcatccaaacaacttgccagaaagtatttgcaaatcctgtagaggggctagagtggttcgaggacagaagtcagtgaaagtcactattgatccaggggttgacaatagtgatacattaaaggtggcaagggtgggtggggctgatcctgaaggtgaccagcctggagatctttatgttactctcaaggttcgtgaagatcctgtgttccgcagagaaggatcggatattcatgtggacgcggttctcagtgttacccagcatctattttggacttctggtgcagtatccgccattcttggaggaaccattcaagttccaaccctcactggtgatgttgtcgtgaaggtccgtcctggaacccaacctggtcacaaagtagtgctaagaaataaaggaattagagcaagaaagtcgactaaatttggggatcagtatgtgcatttcaacgtcagcatccctgcaaatataacgcagagacagcgtgaactgcttgaggaatttagtaaagcagaacaaggtgaatacgagcagcgcacagcaactggatcttcccagtga

SEQ ID NO: 32 - protein, 461 aa, Arabidopsis thaliana
MVPSNGAKVLRLLSRRCLSSSLIQDLANQKLRGVCIGSYRRLNTSVGNHANVIGDYASKSGHDRKWINFGGFNTNFGSTRSFHGTGSSFMSAKDYYSVLGVSKNAQEGEIKKAYYGLAKKLHPDMNKDDPEAETKFQEVSKAYEILKDKEKRDLYDQVGHEAFEQNASGGFPNDQGFGGGGGGGFNPFDIFGSFNGDIFNMYRQDIGGQDVKVLLDLSFMEAVQGCSKTVTFQTEMACNTCGGQGVPPGTKREKCKACNGSGMFHFSDLTEEGYVKHPNNLPESICKSCRGARVVRGQKSVKVTIDPGVDNSDTLKVARVGGADPEGDQPGDLYVTLKVREDPVFRREGSDIHVDAVLSVTQHLFWTSGAVSAILGGTIQVPTLTGDVVVKVRPGTQPGHKVVLRNKGIRARKSTKFGDQYVHFNVSIPANITQRQRELLEEFSKAEQGEYEQRTATGSSQ

SEQ ID NO: 33 - DNA, 1317 bp, Arabidopsis thaliana
  CDS (1)..(1317)
atggctgcaatggctcgctgtgctttgattccatctataaacccagctcatagcttccgtcatcagtttccgcaacccaatgcgtcattctatttacctcccactcttccgattttttcgcgtgttcggagatttggaatttccggcggatatcgccgccgtgtgatcaccatggccgccggaactgatcactactcgactttgaatgtgaaccgcaatgccaccttgcaggagatcaagagctcgtatagaaaactcgctcgcaagtatcacccagatatgaacaagaaccctggtgcagaagataagttcaaacagataagtgctgcttacgaggtattatctgatgaggagaagagatctgcctatgatcggttcggcgaggctggtttagaaggtgattttaatggatcacaggatacttcaccaggggtggatccatttgacttgtacagtgcattctttggaggttctgatggattctttgggggaatgggtgaatcaggagggatgggttttgatttcatgaataagagaagcctagaccttgacattcgatatgacctgcggttgagctttgaagaggcagtttttggagtaaaacgggagattgaggtttcttatttagaaacatgtgatggttgtggaggaactggtgctaaatccagtaactccattaaacagtgtagtagttgtgatggtaaaggacgtgtgatgaattctcagagaacaccctttggaatcatgtctcaggtgtccacttgctccaaatgtggtggtgaaggaaaaactatcactgataagtgtcgaaagtgcattggcaacgggagactacgggctaggaaaaagatggatgttgtagtccctcctggtgtcagcgatagagccacaatgcgaattcaaggagaaggtaacatggacaagagaagtggaagagctggtgacttgttcatcgtacttcaagttgatgagaagcgtggaattcggcgggaagggcttaatttatactccaatatcaacatagatttcacagatgctatacttggggcgaccacaaaggtagaaacggttgagggatcaatggatcttcggattccgccaggaactcaacctggcgacaccgtaaaattacctaggaaaggagttccagacacggatagaccttcaatccggggagaccattgctttgtagtgaaaatttcgatccccaaaaaactgagcgagagggagcgcaagttggtagaggaattctcgtcgctcagaagatctagtagtagtactggacctactggtacaatgctatcccaatctaattga

SEQ ID NO: 34 - protein, 438 aa, Arabidopsis thaliana
MAAMARCALIPSINPAHSFRHQFPQPNASFYLPPTLPIFSRVRRFGISGGYRRRVITMAAGTDHYSTLNVNRNATLQEIKSSYRKLARKYHPDMNKNPGAEDKFKQISAAYEVLSDEEKRSAYDRFGEAGLEGDFNGSQDTSPGVDPFDLYSAFFGGSDGFFGGMGESGGMGFDFMNKRSLDLDIRYDLRLSFEEAVFGVKREIEVSYLETCDGCGGTGAKSSNSIKQCSSCDGKGRVMNSQRTPFGIMSQVSTCSKCGGEGKTITDKCRKCIGNGRLRARKKMDVVVPPGVSDRATMRIQGEGNMDKRSGRAGDLFIVLQVDEKRGIRREGLNLYSNINIDFTDAILGATTKVETVEGSMDLRIPPGTQPGDTVKLPRKGVPDTDRPSIRGDHCFVVKISIPKKLSERERKLVEEFSSLRRSSSSTGPTGTMLSQSN

SEQ ID NO: 35 - DNA, 1191 bp, Arabidopsis thaliana
  CDS (1)..(1191)
atgtttgcacagggctctttacccttcttgtcattgacgggagtatctcctaatacacattctcgtagaggagctcgcttcactgttagagctgatactgatttctattctgtccttggagtctcgaaaaatgcaaccaaagctgagattaaaagcgcttatcggaagctcgctaggagttatcatccagatgtgaacaaggatgctggggcagaagataaatttaaagaaataagtaatgcatatgagatcttatcagatgatgagaaaagatctctatacgacagatatggcgaggcaggagttaaaggcgctggaatgggaggcatgggggattatagtaatccgtttgatctatttgagtcattattcgaaggaatgggtgggatgggaggaatgggcggtggaatgggtagtagaggttcaaggagcagagctatcgatggtgaagatgagtattactcactaatcttgaatttcaaagaagcggttttcggtattgagaaagaaattgagatatctcggttagagagctgtgggacttgcaatggttctggagctaaagcgggaaccaaaccaaccaaatgcaaaacatgtggcgggcaaggacaggtggtagcatcaacgaggacaccactcggtgtattccaacaagtgatgacttgctctccgtgtaacggaactggggagatatcaaaaccgtgtggtgcatgctcaggagatggacgtgtgagaaggacaaagcggattagtcttaaagttcctgcgggtgtggattctggaagtaggttaagagtgaggggagaagggaatgcaggaaagagaggtggatcaccgggagatctctttgcggttattgaggttattccagatccggttttgaagcgtgatgatacaaatatactttatacgtgtaagatatcgtatgtagatgccatattggggacgactttgaaggtaccaacagtggatggagaggtggatttgaaagtaccggcagggacacaaccaagcacgacattggtgatggctaaaaaaggagttccggttttgaataagagcaagatgagaggtgatcagttagtgagagtgcaagttgagattcctaagagattgagtaaagaagagaagatgcttgttgaggagctggctgatatgagcaagaacaaggtagctaatagcaggagataa

SEQ ID NO: 36 - protein, 396 aa, Arabidopsis thaliana
MFAQGSLPFLSLTGVSPNTHSRRGARFTVRADTDFYSVLGVSKNATKAEIKSAYRKLARSYHPDVNKDAGAEDKFKEISNAYEILSDDEKRSLYDRYGEAGVKGAGMGGMGDYSNPFDLFESLFEGMGGMGGMGGGMGSRGSRSRAIDGEDEYYSLILNFKEAVFGIEKEIEISRLESCGTCNGSGAKAGTKPTKCKTCGGQGQVVASTRTPLGVFQQVMTCSPCNGTGEISKPCGACSGDGRVRRTKRISLKVPAGVDSGSRLRVRGEGNAGKRGGSPGDLFAVIEVIPDPVLKRDDTNILYTCKISYVDAILGTTLKVPTVDGEVDLKVPAGTQPSTTLVMAKKGVPVLNKSKMRGDQLVRVQVEIPKRLSKEEKMLVEELADMSKNKVANSRR

SEQ ID NO: 37 - DNA, 1260 bp, Arabidopsis thaliana
  CDS (1)..(1260)
atgtttggaagaggaccttcaaggaagagcgataacacaaagttctacgagatccttggtgttcctaagaccgcagcaccagaagatctcaagaaagcttataagaaagccgctatcaaaaaccatcctgataagggtggtgatcccgaaaagtttaaagagttagcacaggcttatgaagttttaagtgatcctgagaagcgtgagatctatgatcaatatggggaagatgcactcaaggaaggaatgggtggtggaggtggtggacacgatccatttgatatcttctcttccttctttggtagtggtggacacccattcggaagtcatagccggggaaggaggcagaggcgtggtgaagatgttgttcatcccttgaaggtttccttagaggatgtttatctcggaacaacaaagaagctctcactttctaggaaggctttgtgctcaaagtgtaacggcaagggttcaaagtctggagcttcaatgaaatgtggtggctgtcaaggttcgggaatgaagatctcgatcaggcagtttggacctggaatgatgcagcaggtgcagcatgcttgtaatgattgcaaaggcacaggagagaccatcaatgatcgggacaggtgtccacaatgcaaaggagagaaggttgtctctgagaagaaggtgcttgaagtaaatgtggagaagggaatgcaacacaatcagaagatcacattcagtggacaagccgatgaagcgcctgatactgtcaccggagatatagtgtttgtcattcagcagaaggagcacccaaagttcaaaagaaagggtgaggatctctttgtggagcacaccatctctctaaccgaggccttgtgtggcttccagtttgtcttgacccatttggacaaaagacagcttctcatcaaatccaagcccggagaggtcgtcaaacctgattcatacaaggcgataagtgatgagggaatgccaatataccaaaggccgttcatgaagggtaagctatacattcacttcacggttgaattcccggaatcgctgagcccggatcagacaaaggccattgaagcagttttgccaaagccaaccaaggcagctataagcgatatggaaatagacgactgcgaagagacgactctgcatgatgtgaacattgaggatgagatgaaaaggaaggcgcaagctcaaagagaggcttatgatgacgatgaggaagatcacccaggcggtgctcagcgtgtgcaatgtgcccagcagtga

SEQ ID NO: 38 - protein, 419 aa, Arabidopsis thaliana
MFGRGPSRKSDNTKFYEILGVPKTAAPEDLKKAYKKAAIKNHPDKGGDPEKFKELAQAYEVLSDPEKREIYDQYGEDALKEGMGGGGGGHDPFDIFSSFFGSGGHPFGSHSRGRRQRRGEDVVHPLKVSLEDVYLGTTKKLSLSRKALCSKCNGKGSKSGASMKCGGCQGSGMKISIRQFGPGMMQQVQHACNDCKGTGETINDRDRCPQCKGEKVVSEKKVLEVNVEKGMQHNQKITFSGQADEAPDTVTGDIVFVIQQKEHPKFKRKGEDLFVEHTISLTEALCGFQFVLTHLDKRQLLIKSKPGEVVKPDSYKAISDEGMPIYQRPFMKGKLYIHFTVEFPESLSPDQTKAIEAVLPKPTKAAISDMEIDDCEETTLHDVNIEDEMKRKAQAQREAYDDDEEDHPGGAQRVQCAQQ

SEQ ID NO: 39 - DNA, 1263 bp, Arabidopsis thaliana
  CDS (1)..(1263)
atgttcggtagaggaccctcgaagaagagcgacaacactaagttctacgagatcttaggtgttcctaagagcgcttcaccagaagatctcaagaaagcttacaaaaaagccgctatcaagaatcatcctgataagggtggagatcccgagaagtttaaggagttagcacaagcttatgaagtgcttagtgacccggagaagcgtgagatttatgaccagtatggagaggatgcactcaaggaaggaatgggtggtggaggaggtggacatgatccatttgatattttctcatccttctttggtggaggcccctttggaggtaataccagccggcaaaggaggcagaggcgtggtgaggatgttgttcatcccttgaaggtatctcttgaggatgtgtaccttggtacaatgaagaagctttcactttctaggaatgctctctgctctaagtgtaacggaaagggatcaaaatctggagcctccttgaaatgtggagggtgtcagggatctggtatgaaggtgtctattaggcagcttggacctggaatgatccagcagatgcagcatgcatgtaatgaatgcaaagggacaggtgagaccatcaatgatcgggacaggtgtccacaatgcaaaggagacaaggtcattcctgagaagaaggtgcttgaagtgaatgtggagaagggaatgcaacacagtcagaagatcacatttgaaggacaagcagatgaagcgcctgacactgtcactggagatatagtgtttgtccttcagcagaaagagcacccaaagttcaagagaaagggagaagacctctttgtggagcacacactttctctaaccgaagctttgtgtggcttccaatttgttctgactcacttggatggcagaagtcttctcattaaatctaatcctggggaggtcgtgaaacctgattcatacaaggcaataagcgatgaaggaatgccgatataccagaggccattcatgaagggtaagctctacatccacttcacagtggagttcccggactcgttgagcccagatcagaccaaagcactggaagctgttctacctaagccgtcaacagctcagttgagtgacatggagatagatgaatgcgaggagaccacgctccacgatgtcaacattgaggatgagatgaggaggaaggcacaagctcaaagagaggcttatgatgatgacgatgaagatgatgaccatccgggtggtgctcaaagggtgcaatgtgcccagcagtaa

SEQ ID NO: 40 - protein, 420 aa, Arabidopsis thaliana
MFGRGPSKKSDNTKFYEILGVPKSASPEDLKKAYKKAAIKNHPDKGGDPEKFKELAQAYEVLSDPEKREIYDQYGEDALKEGMGGGGGGHDPFDIFSSFFGGGPFGGNTSRQRRQRRGEDVVHPLKVSLEDVYLGTMKKLSLSRNALCSKCNGKGSKSGASLKCGGCQGSGMKVSIRQLGPGMIQQMQHACNECKGTGETINDRDRCPQCKGDKVIPEKKVLEVNVEKGMQHSQKITFEGQADEAPDTVTGDIVFVLQQKEHPKFKRKGEDLFVEHTLSLTEALCGFQFVLTHLDGRSLLIKSNPGEVVKPDSYKAISDEGMPIYQRPFMKGKLYIHFTVEFPDSLSPDQTKALEAVLPKPSTAQLSDMEIDECEETTLHDVNIEDEMRRKAQAQREAYDDDDEDDDHPGGAQRVQCAQQ

SEQ ID NO: 41 - DNA, 1263 bp, Oryza sativa
  CDS (1)..(1263)
atgtacggacgcatgccaaagaagagtaacaataccaagtattatgaggtgcttggtgtatctaagacagcaacccaggatgagctgaagaaagcgtaccgtaaagctgccattaaaaaccaccctgataagggtggagaccctgagaagtttaaagaattggctcaagcttacgaggttcttaatgatcctgaaaagagggaaatctatgaccaatatggcgaggatgcactcaaagaaggaatgggaggaggcagcagcagtgatttccatagtcccttcgatttatttgagcaaatttttcagaatcgtggtggctttgggggtagaggacacagacaaaagcgtggcgaagatgtggtacatactatgaaggtttctttagaagacctgtataatggtactaccaaaaaactgtctttgtcacggaatgctctgtgcacaaagtgcaagggtaaaggatccaagagtggggcagcagcaacttgccatggttgtcatggtgcaggaatgagaacaataacaagacaaattgggcttggcatgatccaacagatgaacactgtttgccctgaatgcagaggatcaggtgagatgataagtgacaaggataaatgcccgagttgtaagggaaacaaagtagtccagcagaagaaggtcttggaggttcatgttgagaagggaatgcaacatggccaaaagattgtattccagggtgaagctgatgaagctcctgatacagtgacaggagacatagtttttgtcttgcaacttaaagaccacccaaaatttaagaggaagtttgatgacctctttactgagcacacaatctccctgaccgaggctctgtgtggcttccagtttgttctaacccatcttgatggtcggcaactcctaatcaaatctaatccaggggaggttataaaacctggtcaacacaaggccatcaatgatgaaggcatgccccagcatggccgccctttcatgaaaggtcgtctttttgttgaattcaacgtggagtttcctgagcctggtgcactcactcctggccaatgccgatcgcttgagaagattttgccaccacgacccaggaatcaattgtcagacatggagctagatcaatgtgaggagaccaccatgcatgatgtcaacatagaagaggagatgaggcgcaggcagcagcacaggcggcaggaagcatatgatgaagacgacgacgaggatgctggagctggaccaagggtacagtgtgcccagcagtaa

SEQ ID NO: 42 - protein, 420 aa, Oryza sativa
MYGRMPKKSNNTKYYEVLGVSKTATQDELKKAYRKAAIKNHPDKGGDPEKFKELAQAYEVLNDPEKREIYDQYGEDALKEGMGGGSSSDFHSPFDLFEQIFQNRGGFGGRGHRQKRGEDVVHTMKVSLEDLYNGTTKKLSLSRNALCTKCKGKGSKSGAAATCHGCHGAGMRTITRQIGLGMIQQMNTVCPECRGSGEMISDKDKCPSCKGNKVVQQKKVLEVHVEKGMQHGQKIVFQGEADEAPDTVTGDIVFVLQLKDHPKFKRKFDDLFTEHTISLTEALCGFQFVLTHLDGRQLLIKSNPGEVIKPGQHKAINDEGMPQHGRPFMKGRLFVEFNVEFPEPGALTPGQCRSLEKILPPRPRNQLSDMELDQCEETTMHDVNIEEEMRRRQQHRRQEAYDEDDDEDAGAGPRVQCAQQ

SEQ ID NO: 43 - DNA, 27 bp, Artificial sequence
  primer
atggttaaagaaactaagttttacgat

SEQ ID NO: 44 - DNA, 25 bp, Artificial sequence
  primer
tcattgagatgcacattgaacacct

SEQ ID NO: 45 - protein, 416 aa, Artificial sequence
  consensus sequence
LGVXXXAXXXXXKXAYRXXAXXXHPDXXXXXXXXXXXXXXXXFKXXXXAYXXLXDXXKRXXYDXXGXXXXXXXXXXXXXXXXXXXXXXXXXXXXXXXXXXXXXXXXXXXXXXXXXXFXXXXXXXXXXXXXXXXXXXXXXXXXXXXXXXXXXXXXXXXXXXXXGXDXXXXXXXXXXXXXXXXXXXXXXXXXXXXXXCXXXXXXXXXXXXXXXXXXXCXXXXXXXXXXXXXXXXXXXXXXXXXXXXXXGXXXXXXXXCXXCXGXXXXXXXXXXXXXXXXXGXXXGXXXXXXXXGXXXXXXXXXXXXXXXXXXXXXXXXXFXRXGXXXXXXXXXXXXXXXXXXXXXXXAXXGXXXXXXXXXXGXXXXXXXXXXXXXXXXXXXXXXXXXXXXXXXXXXXXXGXXXXXXXXXXXXXXXXXG

SEQ ID NO: 46 - protein, 14 aa, Artificial sequence
  protein pattern
KXAXXXXAXXXHPD

SEQ ID NO: 47 - protein, 28 aa, Artificial sequence
  protein pattern
EXXXFKXXXXAYXXLXXXXXXXXXDXXG

SEQ ID NO: 48 - DNA, 8659 bp, Artificial sequence
  plasmid pMTX155
agcttggacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcagggcatgccggtcgagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagactcgacggatccacgtgtggaagatatgaatttttttgagaaactagataagattaatgaatatcggtgttttggttttttcttgtggccgtctttgtttatattgagatttttcaaatcagtgcgcaagacgtgacgtaagtatccgagtcagtttttatttttctactaatttggtcgaagctttgggcggatcctctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacagggtaccctggaattccagctgaccaccatggcaattcccggggatcagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaatta

(19) United States
(12) Patent Application Publication
(10) Pub. No.: US 2013/0340119 A1
(43) Pub. Date: Dec. 19, 2013

(54) Method for Increasing Yield and Fine Chemical Production in Plants

(71) Applicants: Gunnar Plesch, Potsdam (DE); Astrid Blau, Stahnsdorf (DE); Michael Manfred Herold, Berlin (DE); Beate Kamlage, Berlin (DE); Birgit Wendel, Berlin (DE); Piotr Puzio, Mariakerke (Gent) (BE); Oliver Bläsing, Potsdam (DE); Oliver Thimm, Neustadt (DE); Janneke Hendriks, Schwielowsee (DE); Christophe Reuzeau, La Chapelle Gonaguet (FR)

(72) Inventors: Gunnar Plesch, Potsdam (DE); Astrid Blau, Stahnsdorf (DE); Michael Manfred Herold, Berlin (DE); Beate Kamlage, Berlin (DE); Birgit Wendel, Berlin (DE); Piotr Puzio, Mariakerke (Gent) (BE); Oliver Bläsing, Potsdam (DE); Oliver Thimm, Neustadt (DE); Janneke Hendriks, Schwielowsee (DE); Christophe Reuzeau, La Chapelle Gonaguet (FR)

(73) Assignee: BASF Plant Science Company GmbH, Ludwigshafen (DE)

(21) Appl. No.: 13/883,450

(22) PCT Filed: Oct. 27, 2011
(86) PCT No.: PCT/IB11/54792

§ 371 Date: May 3, 2013

Related U.S. Application Data
(60) Provisional application No. 61/485,641, filed on May 13, 2011.

(30) Foreign Application Priority Data
Nov. 5, 2010 (EP) 10190115.5
Nov. 8, 2010 (EP) 10190348.2
Nov. 12, 2010 (EP) 10190974.5
Nov. 18, 2010 (EP) PCT/EP2010/006988
May 13, 2011 (EP) 11165957.9

(51) Int. Cl. C12N 15/82

(52) U.S. Cl. CPC C12N 15/8247; C12N 15/8245
USPC 800/290; 435/100; 435/105; 435/134; 435/419; 435/320.1; 536/128; 554/8; 800/278; 800/298

(57) ABSTRACT

A method for enhancing yield-related traits in plants by modulating expression in a plant of a nucleic acid encoding a POI (Protein Of Interest) polypeptide is provided. Methods for the production of plants having modulated expression of a nucleic acid encoding a DnaJ-like chaperone polypeptide are provided, in which plants have enhanced yield-related traits compared to control plants. Nucleic acids encoding DnaJ-like chaperone, constructs comprising the same and uses thereof are also provided.

The instant application is based on and claims the benefit of prior filed: U.S. provisional application 61/485,641, EP 11165957.9, EP 10190115.5, EP 10190348.2, EP 10190974.5 and the international application WO 2011/060920 (PCT/EP2010/006988). The entire content of the above-referenced patent applications are incorporated herein by this reference, and in particular of EP 10190974.5 page 1431, last paragraph to line 24 of page 1432, page 1935 last paragraph to page 1937, line 20 as well as those lines of tables I, II, IV and d relating to Ynl064c and its related sequences as defined therein, and of the international application WO 2011/060920 (PCT/EP2010/006988) page 5816, lines 9 to 25, page 5878, line 21 to line 8 of the following page, page 6235, lines 9 to 25, page 6301, lines 4 to 34, page 1, line 16 to line 8 of the following page, page 1, line 20 to the last line of the following page as well as those lines of tables d, I, II, IV and relating to Ynl064c, SEQ ID NO: 117495 and related sequences (e.g. homologs, paralogues) as defined therein.

The present invention relates generally to the field of molecular biology and concerns a method for enhancing yield-related traits in plants and/or the production of fine chemicals by modulating expression in a plant of a nucleic acid encoding a POI (Protein Of Interest) polypeptide. The present invention also concerns use of POI polypeptides in plants for having modulated expression of a nucleic acid encoding a POI polypeptide, which plants have enhanced yield-related traits or increased content of fine chemicals relative to corresponding wild type plants or other control plants.

The ever-increasing world population and the dwindling supply of arable land available for agriculture fuels research towards increasing the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits.

A trait is increased yield. Yield is normally defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance and early vigour may also be important factors in determining yield. Optimizing the abovementioned factors may therefore contribute to increasing crop yield.

Seed yield is an important trait, since the seeds of many plants are important for human and animal nutrition. Crops such as corn, rice, wheat, canola and soybean account for over half the total human caloric intake, whether through direct consumption of the seeds themselves or through consumption of meat products raised on processed seeds. They are also a source of sugars, oils and many kinds of metabolites used in industrial processes. Seeds contain an embryo (the source of new shoots and roots) and an endosperm (the source of nutrients for embryo growth during germination and during early growth of seedlings). The development of a seed involves many genes, and requires the transfer of metabolites from the roots, leaves and stems into the growing seed. The endosperm, in particular, assimilates the metabolic precursors of carbohydrates, oils and proteins and synthesizes them into storage macromolecules to fill out the grain.

Another important trait for many crops is early vigour. Improving early vigour is an important objective of modern rice breeding programs in both temperate and tropical rice cultivars. Long roots are important for proper soil anchorage in water-seeded rice. Where rice is sown directly into flooded fields, and where plants must emerge rapidly through water, longer shoots are associated with vigour. Where drill-seeding is practiced, longer mesocotyls and coleoptiles are important for good seedling emergence. The ability to engineer early vigour into plants would be of great importance in agriculture. For example, poor early vigour has been a limitation to the introduction of maize (Zea mays L.) hybrids based on Corn Belt germplasm in the European Atlantic.

A further important trait is that of improved abiotic stress tolerance. Abiotic stress is a primary cause of crop loss worldwide, reducing average yields for most major crop plants by more than 50% (Wang et al., Planta 218, 1-14, 2003). Abiotic stresses may be caused by drought, salinity, extremes of temperature, chemical toxicity and oxidative stress. The ability to improve plant tolerance to abiotic stress would be of great economic advantage to farmers worldwide and would allow for the cultivation of crops during adverse conditions and in territories where cultivation of crops may not otherwise be possible.

Crop yield may therefore be increased by optimising one of the above-mentioned factors.

Depending on the end use, the modification of certain yield traits may be favoured over others. For example for applications such as forage or wood production, or bio-fuel resource, an increase in the vegetative parts of a plant may be desirable, and for applications such as flour, starch or oil production, an increase in seed parameters may be particularly desirable. Even amongst the seed parameters, some may be favoured over others, depending on the application. Various mechanisms may contribute to increasing seed yield, whether that is in the form of increased seed size or increased seed number.

Improving the quality of foodstuffs and animal feeds is an important task of the food-and-feed industry. This is necessary since, for example, certain fatty acids E, which occur in plants are limited with regard to the supply of mammals. Especially advantageous for the quality of foodstuffs and animal feeds is an as balanced as possible fatty acid profile since a great excess of certain fatty acids like omega-3-fatty acids above a specific concentration in the food has no further positive effect unless the omega-3-fatty acid content is in balance to the omega-6-fatty acid content of the diet. A further increase in quality is only possible via addition of further fatty acids, which are limiting under these conditions. The targeted addition of the limiting fatty acid in form of synthetic products must be carried out with extreme caution in order to avoid fatty acid imbalance.

To ensure a high quality of foods and animal feeds, it is therefore necessary to add a plurality of fatty acids in a balanced manner to suit the respective organism. Accordingly, there is still a great demand for new and more suitable genes, which encode enzymes or regulators, which participate in the biosynthesis of fatty acids and make it possible to produce certain fatty acids specifically on an industrial scale without unwanted byproducts being formed. In the selection of genes for biosynthesis or regulation two characteristics above all are particularly important. On the one hand, there is as ever a need for improved processes for obtaining the highest possible contents of fatty acids and on the other hand as less as possible byproducts should be produced in the production process.

Fatty acids are the building blocks of triglycerides, phospholipids, lipids, oils and fats. Some of the fatty acids such as linoleic or linolenic acid are “essential” because the human body is not able to synthesize them but needs them, so humans must ingest them through the diet. The human body can synthesize other fatty acids therefore they are not labeled as “essential”. Nevertheless very often the amount of production of for example fatty acids such as eicosapentaenoic acid (=EPA, C20:5Δ^{5,8,11,14,17}) or docosahexaenoic acid (=DHA, C22:6Δ^{4,7,10,13,16,19}) in the body is not sufficient for an optimal body function. Polyunsaturated fatty acids (=PUFA) that mean fatty acids, which have more than 1 double bond in the carbon chain are divided into families depending on where their end-most double bond is located. There are two main subtypes of fatty acids: the omega-3 and omega-6 fatty acids. The Omega-3's are those with their endmost double bond 3 carbons from their methyl end. The Omega-6's are those with their endmost double bond 6 carbons from their methyl end. Linoleic acid (an omega-6) and alpha-linolenic acid (an omega-3) are the only true “essential” fatty acids. Both are used inside the body as starting material to synthesize others such as EPA or DHA.

Fatty acids and triglycerides have numerous applications in the food and feed industry, in cosmetics and in the drug sector. Depending on whether they are free saturated or unsaturated fatty acids or bound, e.g. in form of triglycerides with an increased content of saturated or unsaturated fatty acids, they are suitable for the most varied applications; thus, for example, polyunsaturated fatty acids (=PUFAs) are added to infant formula to increase its nutritional value. The various fatty acids and triglycerides are mainly obtained from microorganisms such as fungi, from animals such as fish or from oil-producing plants including phytoplankton and algae, such as soybean, oilseed rape, sunflower and others, where they are usually obtained in the form of their triacylglycerides.

It is an object of the present invention to develop an inexpensive process for the synthesis of linoleic acid and/or linolenic acid. Linoleic acid and linolenic acid are two of the fatty acids which are most frequently limiting.

It is an object of the present invention to develop an inexpensive process for the synthesis of sucrose, and/or myo-inositol. It is a further object of the present invention to develop an inexpensive process for the synthesis of saccharides, in particular derivates of monosaccharides e.g. myo-inositol; and/or disaccharides, preferably sucrose and to assure that said saccharides are more accessible and facilely to isolate and recover in an industrial scale from the producing organism, preferably from a plant.

It has now been found that various yield-related traits and/or the production of fine chemicals may be improved in plants by modulating expression in a plant of a nucleic acid encoding a POI (Protein Of Interest) polypeptide in a plant by the processes according to the invention described herein and the embodiments characterized herein as well as in the claims.

### BACKGROUND

DnaJ is a molecular co-chaperone of the Hsp40 family. Hsp40 cooperates with chaperone heat shock protein 70 (Hsp70, also called DnaK) and cochaperone nucleotide exchange factor GrpE to facilitate different aspects of cellular protein metabolism that include ribosome assembly, protein translocation, protein folding and unfolding, suppression of polypeptide aggregation and cell signaling (Walid (2001) Curr Protein Peptide Sci 2: 227-244). DnaJ stimulates Hsp70 to hydrolyze ATP, a key step in the stable binding of a substrate to Hsp70. In addition, DnaJ itself also possesses molecular chaperone functions since it has been shown to bind to nascent chains in vitro translation systems and to prevent the aggregation of denatured polypeptides (Laufen et al. (2001) Proc Natl Acad Sci USA 96: 5452-5457). Members of the DnaJ family have been identified in a variety of organisms (both in prokaryotes and eukaryotes) and in a variety of cellular compartments, such as cytosol, mitochondria, peroxisome, glyoxysome, endoplasmic reticulum and chloroplast stroma. Within one organism, multiple Hsp40s can interact with a single Hsp70 to generate Hsp70::Hsp40 pairs that facilitate numerous reactions in cellular protein metabolism.

All DnaJ proteins are defined by the presence of a so-called “J” domain, consisting of approximately 70 amino acids, usually located at the amino terminus of the protein, and by the presence of the highly conserved HPD tri-peptide in the middle of the J-domain (InterPro reference IPR001623; Zdobnov et al., (2002) 18(8): 1149-50); The “J” domain, consisting of 35 four alpha helices, interacts with Hsp70 proteins. In the genome of Arabidopsis thaliana, at least 89 proteins comprising the J-domain have been identified (Miernyk (2001) Cell Stress & Chaperones).

DnaJ proteins have been further classified into Type I, Type II and Type III.

DnaJ domain proteins (or DnaJ proteins) of type I (Miernyk (2001) Cell Stress & Chaperone 6(3): 209-218), comprise (from amino terminus to carboxy terminus) the domains identified within the archetypal DnaJ protein as first characterized in Escherichia coli:
1) a G/F domain region of about 30 amino acid residues, rich in glycine (G) and phenylalanine (F), which is proposed to regulate target polypeptide specificity;
2) a Cys-rich zinc finger domain containing four repeats of the CXXCXGXG, where X represents a charged or polar residue; these four repeats function in pairs to form zinc binding domain I and II (InterPro reference IPR001305; Linke et al. (2003) J Biol Chem 278(45): 44457-44466); the zinc finger domain is thought to mediate direct protein:protein interactions and more specifically to bind non-native polypeptides to be delivered to Hsp70;
3) a .Carboxy-terminal domain (CTD; InterPro reference IPR002939).

Type II DnaJ domain proteins comprise the J domain located at the amino terminus of the protein, either the G/F domain or the zinc finger 20 domain and a CTD. Type III DnaJ domain proteins comprise only the J domain, which may be located anywhere within the protein.

In their native form, DnaJ proteins may be targeted to a variety of subcellular compartments, in either a soluble or a membrane-bound form. Examples of such subcellular compartments in plants include mitochondria, chloroplasts, peroxisomes, nucleus, cytoplasm and secretory pathway. Signal sequences and transit peptides, usually located at the amino terminus of the nuclear-encoded DnaJ proteins, are responsible for the targeting of these proteins to specific subcellular compartments.

DNAL-like polypeptides have been disclosed to increase yield in plants under non-stress conditions (International publication WO06067236.

It has now been found that preferentially increasing activity in the cytosol of a plant cell of a DnaJ-like chaperone gives plants grown under stress conditions increased yield and/or increased fine chemical content relative to corresponding wild type plants grown under comparable conditions.

### SUMMARY

Surprisingly, it has now been found that modulating expression of a nucleic acid encoding a POI polypeptide as defined herein gives plants having enhanced yield-related traits under stress conditions, preferably under abiotic environmental stress conditions, and/or non-stress conditions, in particular increased yield relative to control plants and/or increases the content of fine chemicals.

According one embodiment, there are provided methods for improving yield-related traits of plants under stress conditions, preferably under abiotic environmental stress conditions as provided herein and/or increasing the production of fine chemicals in plants relative to control plants, comprising modulating expression in a plant of a nucleic acid encoding a POI polypeptide as defined herein.

Accordingly, in one embodiment, the invention relates to a process for the production of at least one fine chemical selected from the group consisting of: linoleic acid, linoleic acid, sucrose and myo-inositol.

The section captions and headings in this specification are for convenience and reference purpose only and should not affect in any way the meaning or interpretation of this specification.

### DEFINITIONS

The following definitions will be used throughout the present specification.

### Polypeptide(s)/Protein(s)

The terms “polypeptide” and “protein” are used interchangeably herein and refer to amino acids in a polymeric form of any length, linked together by peptide bonds.

### Polynucleotide(s)/Nucleic Acid(s)/Nucleic Acid Sequence(s)/Nucleotide Sequence(s)

The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

### Homologue(s)

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

A deletion refers to removal of one or more amino acids from a protein.

An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag•100 epitope, c-myc epitope, FLAG®-epitope, lacZ, CMP (calmodulin-binding peptide), HA epitope, protein C epitope and VSV epitope.

A substitution refers to replacement of amino acids of the protein with other amino acids having similar properties (such as similar hydrophobicity, hydrophilicity, antigenicity, propensity to form or break α-helical structures or β-sheet structures). Amino acid substitutions are typically of single residues, but may be clustered depending upon functional constraints placed upon the polypeptide and may range from 1 to 10 amino acids; insertions will usually be of the order of about 1 to 10 amino acid residues. The amino acid substitutions are preferably conservative amino acid substitutions. Conservative substitution tables are well known in the art (see for example Creighton (1984) Proteins. W.H. Freeman and Company (Eds) and Table 1 below).

TABLE 1
Examples of conserved amino acid substitutions

| Residue | Conservative Substitutions |
|---|---|
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

### Derivatives

“Derivatives” include peptides, oligopeptides, polypeptides which may, compared to the amino acid sequence of the naturally-occurring form of the protein, such as the protein of interest, comprise substitutions of amino acids with non-naturally occurring amino acid residues, or additions of non-naturally occurring amino acid residues. “Derivatives” of a protein also encompass peptides, oligopeptides, polypeptides which comprise naturally occurring altered (glycosylated, acylated, prenylated, phosphorylated, myristoylated, sulphated etc.) or non-naturally altered amino acid residues compared to the amino acid sequence of a naturally-occurring form of the polypeptide. A derivative may also comprise one or more non-amino acid substituents or additions compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein. Furthermore, “derivatives” also include fusions of the naturally-occurring form of the protein with tagging peptides such as FLAG, HIS6 or thioredoxin (for a review of tagging peptides, see Terpe, Appl. Microbiol. Biotechnol. 60, 523-533, 2003).

### Orthologue(s)/Paralogue(s)

Orthologues and paralogues encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene.

### Domain, Motif/Consensus Sequence/Signature

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide family.

The term “motif” or “consensus sequence” or “signature” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain).

Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004)), or Pfam (Bateman et al., Nucleic Acids Research 30(1): 276-280 (2002) & The Pfam protein families database: R. D. Finn, J. Mistry, J. Tate, P. Coggill, A. Heger, J. E. Pollington, O. L. Gavin, P. Gunesekaran, G. Ceric, K. Forslund, L. Holm, E. L. Sonnhammer, S. R. Eddy, A. Bateman Nucleic Acids Research (2010) Database Issue 38:D211-222). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003)). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol. 147(1); 195-7).

### Reciprocal BLAST

Typically, this involves a first BLAST involving BLASTing a query sequence (for example using any of the sequences listed in Table A of the Examples section) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived. The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

### Hybridisation

The term “hybridisation” as defined herein is a process wherein substantially homologous complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore medium stringency hybridisation conditions may sometimes be needed to identify such nucleic acid molecules.

The Tₘ is the temperature under defined ionic strength and pH, at which 50% of the target sequence hybridises to a perfectly matched probe. The Tₘ is dependent upon the solution conditions and the base composition and length of the probe. For example, longer sequences hybridise specifically at higher temperatures. The maximum rate of hybridisation is obtained from about 16° C. up to 32° C. below Tₘ. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tₘ decreases about 1° C. per % base mismatch. The Tₘ may be calculated using the following equations, depending on the types of hybrids:

1) DNA-DNA Hybrids (Meinkoth and Wahl, Anal. Biochem., 138: 267-284, 1984):

Tₘ=81.5° C.+16.6×log₁₀ [Na⁺]^{a}+0.41x %[G/C^{b}]−500x[L^{c}]⁻¹−0.61x % formamide

### 2) DNA-RNA or RNA-RNA Hybrids:

Tₘ=79.8° C.+18.5(log₁₀ [Na⁺]^{a})+0.58(% G/C^{b})+11.8(% G/C^{b})²−820/L^{c}

### 3) Oligo-DNA or Oligo-RNAs Hybrids:

For <20 nucleotides: Tₘ=2(lₙ)

For 20-35 nucleotides: Tₘ=22+1.46(lₙ)

^{a} or for other monovalent cation, but only accurate in the 0.01-0.4 M range.
^{b} only accurate for % GC in the 30% to 75% range.
^{c} L=length of duplex in base pairs.
^{d} oligo, oligonucleotide; lₙ, =effective length of primer=2×(no. of G/C)+(no. of NT).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-homologous probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate.

For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

### Splice Variant

The term “splice variant” as used herein encompasses variants of a nucleic acid sequence in which selected introns and/or exons have been excised, replaced, displaced or added, or in which introns have been shortened or lengthened. Such variants will be ones in which the biological activity of the protein is substantially retained; this may be achieved by selectively retaining functional segments of the protein. Such splice variants may be found in nature or may be manmade. Methods for predicting and isolating such splice variants are well known in the art (see for example Foissac and Schiex (2005) BMC Bioinformatics 6: 25).

### Allelic Variant

Alleles or allelic variants are alternative forms of a given gene, located at the same chromosomal position. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

### Endogenous Gene

Reference herein to an “endogenous” gene not only refers to the gene in question as found in a plant in its natural form (i.e., without there being any human intervention), but also refers to that same gene (or a substantially homologous nucleic acid/gene) in an isolated form subsequently (re)introduced into a plant (a transgene). For example, a transgenic plant containing such a transgene may encounter a substantial reduction of the transgene expression and/or substantial reduction of expression of the endogenous gene. The isolated gene may be isolated from an organism or may be manmade, for example by chemical synthesis.

### Gene Shuffling/Directed Evolution

Gene shuffling or directed evolution consists of iterations of DNA shuffling followed by appropriate screening and/or selection to generate variants of nucleic acids or portions thereof encoding proteins having a modified biological activity (Castle et al., (2004) Science 304(5674): 1151-4; U.S. Pat. Nos. 5,811,238 and 6,395,547).

### Construct

Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein. The marker genes may be removed or excised from the transgenic cell once they are no longer needed. Techniques for marker removal are known in the art, useful techniques are described above in the definitions section.

### Regulatory Element/Control Sequence/Promoter

The terms “regulatory element”, “control sequence” and “promoter” are all used interchangeably herein and are to be taken in a broad context to refer to regulatory nucleic acid sequences capable of effecting expression of the sequences to which they are ligated. The term “promoter” typically refers to a nucleic acid control sequence located upstream from the transcriptional start of a gene and which is involved in recognising and binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ.

A “plant promoter” comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule must, as described above, be linked operably to or comprise a suitable promoter which expresses the gene at the right point in time and with the required spatial expression pattern.

For the identification of functionally equivalent promoters, the promoter strength and/or expression pattern of a candidate promoter may be analysed for example by operably linking the promoter to a reporter gene and assaying the expression level and pattern of the reporter gene in various tissues of the plant. Suitable well-known reporter genes include for example beta-glucuronidase or beta-galactosidase. The promoter activity is assayed by measuring the enzymatic activity of the beta-glucuronidase or beta-galactosidase. The promoter strength and/or expression pattern may then be compared to that of a reference promoter (such as the one used in the methods of the present invention). Alternatively, promoter strength may be assayed by quantifying mRNA levels or by comparing mRNA levels of the nucleic acid used in the methods of the present invention, with mRNA levels of housekeeping genes such as 18S rRNA, using methods known in the art, such as Northern blotting with densitometric analysis of autoradiograms, quantitative real-time PCR or RT-PCR (Heid et al., 1996 Genome Methods 6: 986-994). Generally by “weak promoter” is intended a promoter that drives expression of a coding sequence at a low level. By “low level” is intended at levels of about 1/10,000 transcripts to about 1/100,000 transcripts, to about 1/500,0000 transcripts per cell. Conversely, a “strong promoter” drives expression of a coding sequence at high level, or at about 1/10 transcripts to about 1/100 transcripts to about 1/1000 transcripts per cell. Generally, by “medium strength promoter” is intended a promoter that drives expression of a coding sequence at a lower level than a strong promoter, in particular at a level that is in all instances below that obtained when under the control of a 35S CaMV promoter.

### Operably Linked

The term “operably linked” as used herein refers to a functional linkage between the promoter sequence and the gene of interest, such that the promoter sequence is able to initiate transcription of the gene of interest.

### Constitutive Promoter

A “constitutive promoter” refers to a promoter that is transcriptionally active during most, but not necessarily all, phases of growth and development and under most environmental conditions, in at least one cell, tissue or organ. Table 2a below gives examples of constitutive promoters.

TABLE 2a
Examples of constitutive promoters

| Gene Source | Reference |
|---|---|
| Actin | McElroy et al, Plant Cell, 2: 163-171, 1990 |
| HMGP | WO 2004/070039 |
| CAMV 35S | Odell et al, Nature, 313: 810-812, 1985 |
| CaMV 19S | Nilsson et al., Physiol. Plant. 100: 456-462, 1997 |
| GOS2 | de Pater et al, Plant J Nov; 2(6): 837-44, 1992, |
| | WO 2004/065596 |
| Ubiquitin | Christensen et al, Plant Mol. Biol. 18: 675-689, 1992 |
| Rice cyclophilin | Buchholz et al, Plant Mol Biol. 25(5): 837-43, 1994 |
| Maize H3 histone | Lepetit et al, Mol. Gen. Genet. 231: 276-285, 1992 |
| Alfalfa H3 | Wu et al. Plant Mol. Biol. 11: 641-649, 1988 |
| histone | |
| Actin 2 | An et al, Plant J. 10(1); 107-121, 1996 |
| 34S FMV | Sanger et al., Plant. Mol. Biol., 14, 1990: 433-443 |
| Rubisco small | U.S. Pat. No. 4,962,028 |
| subunit | |
| OCS | Leisner (1988) Proc Natl Acad Sci USA 85(5): 2553 |
| SAD1 | Jain et al., Crop Science, 39 (6), 1999: 1696 |
| SAD2 | Jain et al., Crop Science, 39 (6), 1999: 1696 |
| nos | Shaw et al. (1984) Nucleic Acids Res. 12(20): |
| | 7831-7846 |
| V-ATPase | WO 01/14572 |
| Super promoter | WO 95/14098 |
| G-box proteins | WO 94/12015 |

### Ubiquitous Promoter

A ubiquitous promoter is active in substantially all tissues or cells of an organism.

### Developmentally-Regulated Promoter

A developmentally-regulated promoter is active during certain developmental stages or in parts of the plant that undergo developmental changes.

### Inducible Promoter

An inducible promoter has induced or increased transcription initiation in response to a chemical (for a review see Gatz 1997, Annu. Rev. Plant Physiol. Plant Mol. Biol., 48:89-108), environmental or physical stimulus, or may be “stress-inducible”, i.e. activated when a plant is exposed to various stress conditions, or a “pathogen-inducible” i.e. activated when a plant is exposed to exposure to various pathogens.

### Organ-Specific/Tissue-Specific Promoter

An organ-specific or tissue-specific promoter is one that is capable of preferentially initiating transcription in certain organs or tissues, such as the leaves, roots, seed tissue etc. For example, a “root-specific promoter” is a promoter that is transcriptionally active predominantly in plant roots, substantially to the exclusion of any other parts of a plant, whilst still allowing for any leaky expression in these other plant parts. Promoters able to initiate transcription in certain cells only are referred to herein as “cell-specific”.

Examples of root-specific promoters are listed in Table 2b below:

TABLE 2b
Examples of root-specific promoters

| Gene Source | Reference |
|---|---|
| RCc3 | Plant Mol Biol. 1995 January; 27(2): 237-48 |
| Arabidopsis PHT1 | Koyama et al., J Biosci Bioeng. 2005; January; |
| | 99(1): 38-42.; Mudge et al. (2002, Plant |
| | J. 31: 341) |
| Medicago phosphate | Xiao et al., 2006, Plant Biol (Stuttg). 2006 |
| transporter | July; 8(4): 439-49 |
| Arabidopsis Pyk10 | Nitz et al. (2001) Plant Sci 161(2): 337-346 |
| root-expressible genes | Tingey et al., EMBO J. 6: 1, 1987. |
| tobacco auxin- | Van der Zaal et al., Plant Mol. Biol. 16, |
| inducible gene | 983, 1991. |
| β-tubulin | Oppenheimer, et al., Gene 63: 87, 1988. |
| tobacco root- | Conkling, et al., Plant Physiol. 93: 1203, 1990. |
| specific genes | |
| B. napus G1-3b gene | U.S. Pat. No. 5,401,836 |
| SbPRP1 | Suzuki et al., Plant Mol. Biol. 21: 109-119, |
| | 1993. |
| LRX1 | Baumberger et al. 2001, Genes & Dev. 15: 1128 |
| BTG-26 Brassica | US 20050044585 |
| napus | |
| LeAMT1 (tomato) | Lauter et al. (1996, PNAS 3: 8139) |
| The LeNRT1-1 | Lauter et al. (1996, PNAS 3: 8139) |
| (tomato) | |
| class I patatin | Liu et al., Plant Mol. Biol. 17 (6): 1139-1154 |
| gene (potato) | |
| KDC1 (Daucus | Downey et al. (2000, J. Biol. Chem. 275: 39420) |
| carota) | |
| TobRB7 gene | W Song (1997) PhD Thesis, North Carolina State |
| | University, Raleigh, NC USA |
| OsRAB5a (rice) | Wang et al. 2002, Plant Sci. 163: 273 |
| ALF5 (Arabidopsis) | Diener et al. (2001, Plant Cell 13: 1625) |
| NRT2; 1Np (N. | Quesada et al. (1997, Plant Mol. Biol. 34: 265) |
| plumbaginifolia) | |

A seed-specific promoter is transcriptionally active predominantly in seed tissue, but not necessarily exclusively in seed tissue (in cases of leaky expression). The seed-specific promoter may be active during seed development and/or during germination. The seed specific promoter may be endosperm/aleurone/embryo specific. Examples of seed-specific promoters (endosperm/aleurone/embryo specific) are shown in Table 2c to Table 2f below. Further examples of seed-specific promoters are given in Qing Qu and Takaiwa (Plant Biotechnol. J. 2, 113-125, 2004), which disclosure is incorporated by reference herein as if fully set forth.

TABLE 2c
Examples of seed-specific promoters

| Gene source | Reference |
|---|---|
| seed-specific genes | Simon et al., Plant Mol. Biol. 5: 191, 1985; |
| | Scofield et al., J. Biol. Chem. 262: 12202, 1987.; |
| | Baszczynski et al., Plant Mol. Biol. 14: 633, 1990. |
| Brazil Nut albumin | Pearson et al., Plant Mol. Biol. 18: 235-245, 1992. |
| legumin | Ellis et al., Plant Mol. Biol. 10: 203-214, 1988. |
| glutelin (rice) | Takaiwa et al., Mol. Gen. Genet. 208: 15-22, 1986; |
| | Takaiwa et al., FEBS Letts. 221: 43-47, 1987. |
| zein | Matzke et al Plant Mol Biol, 14(3): 323-32 1990 |
| napA | Stalberg et al, Planta 199: 515-519, 1996. |
| wheat LMW and HMW | Mol Gen Genet 216: 81-90, 1989; NAR 17: 461-2, 1989 |
| glutenin-1 | |
| wheat SPA | Albani et al, Plant Cell, 9: 171-184, 1997 |
| wheat α, β, γ-gliadins | EMBO J. 3: 1409-15, 1984 |
| barley Itr1 promoter | Diaz et al. (1995) Mol Gen Genet 248(5): 592-8 |
| barley B1, C, D, hordein | Theor Appl Gen 98: 1253-62, 1999; Plant J 4: 343-55, |
| | 1993; Mol Gen Genet 250: 750-60, 1996 |
| barley DOF | Mena et al, The Plant Journal, 116(1): 53-62, 1998 |
| blz2 | EP99106056.7 |
| synthetic promoter | Vicente-Carbajosa et al., Plant J. 13: 629-640, 1998. |
| rice prolamin NRP33 | Wu et al, Plant Cell Physiology 39(8) 885-889, 1998 |
| rice a-globulin Glb-1 | Wu et al, Plant Cell Physiology 39(8) 885-889, 1998 |
| rice OSH1 | Sato et al, Proc. Natl. Acad. Sci. USA, 93: 8117-8122, |
| | 1996 |
| rice α-globulin REB/OHP-1 | Nakase et al. Plant Mol. Biol. 33: 513-522, 1997 |
| rice ADP-glucose pyrophos- | Trans Res 6: 157-68, 1997 |
| phorylase | |
| maize ESR gene family | Plant J 12: 235-46, 1997 |
| sorghum α-kafirin | DeRose et al., Plant Mol. Biol 32: 1029-35, 1996 |
| KNOX | Postma-Haarsma et al, Plant Mol. Biol. 39: 257-71, 1999 |
| rice oleosin | Wu et al, J. Biochem. 123: 386, 1998 |
| sunflower oleosin | Cummins et al., Plant Mol. Biol. 19: 873-876, 1992 |
| PRO0117, putative rice 40S | WO 2004/070039 |
| ribosomal protein | |
| PRO0136, rice alanine | unpublished |
| aminotransferase | |
| PRO0147, trypsin inhibitor | unpublished |
| ITR1 (barley) | |
| PRO0151, rice WSI18 | WO 2004/070039 |
| PRO0175, rice RAB21 | WO 2004/070039 |
| PRO005 | WO 2004/070039 |
| PRO0095 | WO 2004/070039 |
| α-amylase (Amy32b) | Lanahan et al, Plant Cell 4: 203-211, 1992; Skriver et al, |
| | Proc Natl Acad Sci USA 88: 7266-7270, 1991 |
| cathepsin β-like gene | Cejudo et al, Plant Mol Biol 20: 849-856, 1992 |
| Barley Ltp2 | Kalla et al., Plant J. 6: 849-60, 1994 |
| Chi26 | Leah et al., Plant J. 4: 579-89, 1994 |
| Maize B-Peru | Selinger et al., Genetics 149; 1125-38, 1998 |

TABLE 2d
examples of endosperm-specific promoters

| Gene source | Reference |
|---|---|
| glutelin (rice) | Takaiwa et al. (1986) Mol Gen Genet 208: 15-22; |
| | Takaiwa et al. (1987) FEBS Letts. 221: 43-47 |
| zein | Matzke et al., (1990) Plant Mol Biol 14(3): 323-32 |
| wheat LMW | Colot et al. (1989) Mol Gen Genet 216: 81-90, |
| and HMW | Anderson et al. (1989) NAR 17: 461-2 |
| glutenin-1 | |
| wheat SPA | Albani et al. (1997) Plant Cell 9: 171-184 |
| wheat gliadins | Rafalski et al. (1984) EMBO 3: 1409-15 |
| barley Itr1 | Diaz et al. (1995) Mol Gen Genet 248(5): 592-8 |
| promoter | |
| barley B1, C, D, | Cho et al. (1999) Theor Appl Genet 98: 1253-62; |
| hordein | Muller et al. (1993) Plant J 4: 343-55; |
| | Sorenson et al. (1996) Mol Gen Genet 250: 750-60 |
| barley DOF | Mena et al, (1998) Plant J 116(1): 53-62 |
| blz2 | Onate et al. (1999) J Biol Chem 274(14): 9175-82 |
| synthetic promoter | Vicente-Carbajosa et al. (1998) Plant J 13: 629-640 |
| rice prolamin | Wu et al, (1998) Plant Cell Physiol 39(8) 885-889 |
| NRP33 | |
| rice globulin | Wu et al. (1998) Plant Cell Physiol 39(8) 885-889 |
| Glb-1 | |
| rice globulin | Nakase et al. (1997) Plant Molec Biol 33: 513-522 |
| REB/OHP-1 | |
| rice ADP-glucose | Russell et al. (1997) Trans Res 6: 157-68 |
| pyrophosphorylase | |
| maize ESR | Opsahl-Ferstad et al. (1997) Plant J 12: 235-46 |
| gene family | |
| sorghum kafirin | DeRose et al. (1996) Plant Mol Biol 32: 1029-35 |

TABLE 2e
Examples of embryo specific promoters:

| Gene source | Reference |
|---|---|
| rice OSH1 | Sato et al, Proc. Natl. Acad. Sci. USA, 93: 8117-8122, |
| | 1996 |
| KNOX | Postma-Haarsma et al, Plant Mol. Biol. 39: 257-71, 1999 |
| PRO0151 | WO 2004/070039 |
| PRO0175 | WO 2004/070039 |
| PRO005 | WO 2004/070039 |
| PRO0095 | WO 2004/070039 |

TABLE 2f
Examples of aleurone-specific promoters:

| Gene source | Reference |
|---|---|
| α-amylase | Lanahan et al, Plant Cell 4: 203-211, 1992; |
| (Amy32b) | Skriver et al, Proc Natl Acad Sci USA 88: |
| | 7266-7270, 1991 |
| cathepsin β-like gene | Cejudo et al, Plant Mol Biol 20: 849-856, 1992 |
| Barley Ltp2 | Kalla et al., Plant J. 6: 849-60, 1994 |
| Chi26 | Leah et al., Plant J. 4: 579-89, 1994 |
| Maize B-Peru | Selinger et al., Genetics 149; 1125-38, 1998 |

A green tissue-specific promoter as defined herein is a promoter that is transcriptionally active predominantly in green tissue, substantially to the exclusion of any other parts of a plant, whilst still allowing for any leaky expression in these other plant parts.

Examples of green tissue-specific promoters which may be used to perform the methods of the invention are shown in Table 2g below.

TABLE 2g
Examples of green tissue-specific promoters

| Gene | Expression | Reference |
|---|---|---|
| Maize Orthophosphate dikinase | Leaf specific | Fukavama et al., Plant Physiol. |
| | | 2001 November; 127(3): 1136-46 |
| Maize Phosphoenolpyruvate | Leaf specific | Kausch et al., Plant Mol Biol. |
| carboxylase | | 2001 January; 45(1): 1-15 |
| Rice Phosphoenolpyruvate | Leaf specific | Lin et al., 2004 DNA Seq. |
| carboxylase | | 2004 August; 15(4): 269-76 |
| Rice small subunit Rubisco | Leaf specific | Nomura et al., Plant Mol. Biol. |
| | | 2000 September; 44(1): 99-106 |
| rice beta expansin EXBP9 | Shoot specific | WO 2004/070039 |
| Pigeonpea small subunit Rubisco | Leaf specific | Panguluri et al., Indian J Exp |
| | | Biol. 2005 April; 43(4): 369-72 |
| Pea RBCS3A | Leaf specific | |

Another example of a tissue-specific promoter is a meristem-specific promoter, which is transcriptionally active predominantly in meristematic tissue, substantially to the exclusion of any other parts of a plant, whilst still allowing for any leaky expression in these other plant parts. Examples of green meristem-specific promoters which may be used to perform the methods of the invention are shown in Table 2h below.

TABLE 2h
Examples of meristem-specific promoters

| Gene source | Expression pattern | Reference |
|---|---|---|
| rice OSH1 | Shoot apical meristem, | Sato et al. (1996) |
| | from embryo globular | Proc. Natl. Acad. Sci. |
| | stage to seedling stage | USA, 93: 8117-8122 |
| Rice metallothionein | Meristem specific | BAD87835.1 |
| WAK1 & WAK 2 | Shoot and root apical | Wagner & Kohorn |
| | meristems, and in ex- | (2001) Plant Cell |
| | panding leaves and sepals | 13(2): 303-318 |

### Terminator

The term “terminator” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

### Selectable Marker (Gene)/Reporter Gene

“Selectable marker”, “selectable marker gene” or “reporter gene” includes any gene that confers a phenotype on a cell in which it is expressed to facilitate the identification and/or selection of cells that are transfected or transformed with a nucleic acid construct of the invention. These marker genes enable the identification of a successful transfer of the nucleic acid molecules via a series of different principles. Suitable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin, or genes conferring resistance to, for example, bleomycin, streptomycin, tetracyclin, chloramphenicol, ampicillin, gentamycin, geneticin (G418), spectinomycin or blasticidin), to herbicides (for example bar which provides resistance to Basta®; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilisation of xylose, or antinutritive markers such as the resistance to 2-deoxyglucose). Expression of visual marker genes results in the formation of colour (for example β-glucuronidase, GUS or 3-galactosidase with its coloured substrates, for example X-Gal), luminescence (such as the luciferin/luceferase system) or fluorescence (Green Fluorescent Protein, GFP, and derivatives thereof). This list represents only a small number of possible markers. The skilled worker is familiar with such markers. Different markers are preferred, depending on the organism and the selection method.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid can be identified for example by selection (for example, cells which have integrated the selectable marker survive whereas the other cells die).

Since the marker genes, particularly genes for resistance to antibiotics and herbicides, are no longer required or are undesired in the transgenic host cell once the nucleic acids have been introduced successfully, the process according to the invention for introducing the nucleic acids advantageously employs techniques which enable the removal or excision of these marker genes. One such a method is what is known as co-transformation. The co-transformation method employs two vectors simultaneously for the transformation, one vector bearing the nucleic acid according to the invention and a second bearing the marker gene(s). A large proportion of transformants receives or, in the case of plants, comprises (up to 40% or more of the transformants), both vectors. In case of transformation with Agrobacteria, the transformants usually receive only a part of the vector, i.e. the sequence flanked by the T-DNA, which usually represents the expression cassette. The marker genes can subsequently be removed from the transformed plant by performing crosses. In another method, marker genes integrated into a transposon are used for the transformation together with desired nucleic acid (known as the Ac/Ds technology). The transformants can be crossed with a transposase source or the transformants are transformed with a nucleic acid construct conferring expression of a transposase, transiently or stable. In some cases (approx. 10%), the transposon jumps out of the genome of the host cell once transformation has taken place successfully and is lost. In a further number of cases, the transposon jumps to a different location. In these cases the marker gene must be eliminated by performing crosses. In microbiology, techniques were developed which make possible, or facilitate, the detection of such events. A further advantageous method relies on what is known as recombination systems; whose advantage is that elimination by crossing can be dispensed with. The best-known system of this type is what is known as the Cre/lox system. Cre1 is a recombinase that removes the sequences located between the loxP sequences. If the marker gene is integrated between the loxP sequences, it is removed once transformation has taken place successfully, by expression of the recombinase. Further recombination systems are the HIN/HIX, FLP/FRT and REP/STB system (Tribble et al., J. Biol. Chem., 275, 2000: 22255-22267; Velmurugan et al., J. Cell Biol., 149, 2000: 553-566). A site-specific integration into the plant genome of the nucleic acid sequences according to the invention is possible. Naturally, these methods can also be applied to microorganisms such as yeast, fungi or bacteria.

### Transgenic/Transgene/Recombinant

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either
(a) the nucleic acid sequences encoding proteins useful in the methods of the invention, or
(b) genetic control sequence(s) which is operably linked with the nucleic acid sequence according to the invention, for example a promoter, or
(c) a) and b)
   are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815.

A transgenic plant for the purposes of the invention is thus understood as meaning, as above, that the nucleic acids used in the method of the invention are not present in, or originating from, the genome of said plant, or are present in the genome of said plant but not at their natural locus in the genome of said plant, it being possible for the nucleic acids to be expressed homologously or heterologously. However, as mentioned, transgenic also means that, while the nucleic acids according to the invention or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. Preferred transgenic plants are mentioned herein.

It shall further be noted that in the context of the present invention, the term “isolated nucleic acid” or “isolated polypeptide” may in some instances be considered as a synonym for a “recombinant nucleic acid” or a “recombinant polypeptide”, respectively and refers to a nucleic acid or polypeptide that is not located in its natural genetic environment and/or that has been modified by recombinant methods.

In one embodiment of the invention an “isolated” nucleic acid sequence is located in a non-native chromosomal surrounding.

### Modulation

The term “modulation” means in relation to expression or gene expression, a process in which the expression level is changed by said gene expression in comparison to the control plant, the expression level may be increased or decreased. The original, unmodulated expression may be of any kind of expression of a structural RNA (rRNA, tRNA) or mRNA with subsequent translation. For the purposes of this invention, the original unmodulated expression may also be absence of any expression. The term “modulating the activity” or the term “modulating expression” shall mean any change of the expression of the inventive nucleic acid sequences or encoded proteins, which leads to increased yield and/or increased growth of the plants. The expression can increase from zero (absence of, or immeasurable expression) to a certain amount, or can decrease from a certain amount to immeasurable small amounts or zero.

### Expression

The term “expression” or “gene expression” means the transcription of a specific gene or specific genes or specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product.

### Increased Expression/Overexpression

The term “increased expression” or “overexpression” as used herein means any form of expression that is additional to the original wild-type expression level. For the purposes of this invention, the original wild-type expression level might also be zero, i.e. absence of expression or immeasurable expression.

Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the polypeptide of interest. For example, endogenous promoters may be altered in vivo by mutation, deletion, and/or substitution (see, Kmiec, U.S. Pat. No. 5,565,350; Zarling et al., WO9322443), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell. biol. 8: 4395-4405; Callis et al. (1987) Genes Dev 1:1183-1200). Such intron enhancement of gene expression is typically greatest when placed near the 5′ end of the transcription unit. Use of the maize introns Adh1-S intron 1, 2, and 6, the Bronze-1 intron are known in the art. For general information see: The Maize Handbook, Chapter 116, Freeling and Walbot, Eds., Springer, N.Y. (1994).

### Decreased Expression

Reference herein to “decreased expression” or “reduction or substantial elimination” of expression is taken to mean a decrease in endogenous gene expression and/or polypeptide levels and/or polypeptide activity relative to control plants. The reduction or substantial elimination is in increasing order of preference at least 10%, 20%, 30%, 40% or 50%, 60%, 70%, 80%, 85%, 90%, or 95%, 96%, 97%, 98%, 99% or more reduced compared to that of control plants.

For the reduction or substantial elimination of expression an endogenous gene in a plant, a sufficient length of substantially contiguous nucleotides of a nucleic acid sequence is required. In order to perform gene silencing, this may be as little as 20, 19, 18, 17, 16, 15, 14, 13, 12, 11, 10 or fewer nucleotides, alternatively this may be as much as the entire gene (including the 5′ and/or 3′ UTR, either in part or in whole). The stretch of substantially contiguous nucleotides may be derived from the nucleic acid encoding the protein of interest (target gene), or from any nucleic acid capable of encoding an orthologue, paralogue or homologue of the protein of interest. Preferably, the stretch of substantially contiguous nucleotides is capable of forming hydrogen bonds with the target gene (either sense or antisense strand), more preferably, the stretch of substantially contiguous nucleotides has, in increasing order of preference, 50%, 60%, 70%, 80%, 85%, 90%, 95%, 96%, 97%, 98%, 99%, 100% sequence identity to the target gene (either sense or antisense strand). A nucleic acid sequence encoding a (functional) polypeptide is not a requirement for the various methods discussed herein for the reduction or substantial elimination of expression of an endogenous gene.

This reduction or substantial elimination of expression may be achieved using routine tools and techniques. A preferred method for the reduction or substantial elimination of endogenous gene expression is by introducing and expressing in a plant a genetic construct into which the nucleic acid (in this case a stretch of substantially contiguous nucleotides derived from the gene of interest, or from any nucleic acid capable of encoding an orthologue, paralogue or homologue of any one of the protein of interest) is cloned as an inverted repeat (in part or completely), separated by a spacer (non-coding DNA).

In such a preferred method, expression of the endogenous gene is reduced or substantially eliminated through RNA-mediated silencing using an inverted repeat of a nucleic acid or a part thereof (in this case a stretch of substantially contiguous nucleotides derived from the gene of interest, or from any nucleic acid capable of encoding an orthologue, paralogue or homologue of the protein of interest), preferably capable of forming a hairpin structure. The inverted repeat is cloned in an expression vector comprising control sequences. A non-coding DNA nucleic acid sequence (a spacer, for example a matrix attachment region fragment (MAR), an intron, a polylinker, etc.) is located between the two inverted nucleic acids forming the inverted repeat. After transcription of the inverted repeat, a chimeric RNA with a self-complementary structure is formed (partial or complete). This double-stranded RNA structure is referred to as the hairpin RNA (hpRNA). The hpRNA is processed by the plant into siRNAs that are incorporated into an RNA-induced silencing complex (RISC). The RISC further cleaves the mRNA transcripts, thereby substantially reducing the number of mRNA transcripts to be translated into polypeptides. For further general details see for example, Grierson et al. (1998) WO 98/53083; Waterhouse et al. (1999) WO 99/53050).

Performance of the methods of the invention does not rely on introducing and expressing in a plant a genetic construct into which the nucleic acid is cloned as an inverted repeat, but any one or more of several well-known “gene silencing” methods may be used to achieve the same effects.

One such method for the reduction of endogenous gene expression is RNA-mediated silencing of gene expression (downregulation). Silencing in this case is triggered in a plant by a double stranded RNA sequence (dsRNA) that is substantially similar to the target endogenous gene. This dsRNA is further processed by the plant into about 20 to about 26 nucleotides called short interfering RNAs (siRNAs). The siRNAs are incorporated into an RNA-induced silencing complex (RISC) that cleaves the mRNA transcript of the endogenous target gene, thereby substantially reducing the number of mRNA transcripts to be translated into a polypeptide. Preferably, the double stranded RNA sequence corresponds to a target gene.

Another example of an RNA silencing method involves the introduction of nucleic acid sequences or parts thereof (in this case a stretch of substantially contiguous nucleotides derived from the gene of interest, or from any nucleic acid capable of encoding an orthologue, paralogue or homologue of the protein of interest) in a sense orientation into a plant. “Sense orientation” refers to a DNA sequence that is homologous to an mRNA transcript thereof. Introduced into a plant would therefore be at least one copy of the nucleic acid sequence. The additional nucleic acid sequence will reduce expression of the endogenous gene, giving rise to a phenomenon known as co-suppression. The reduction of gene expression will be more pronounced if several additional copies of a nucleic acid sequence are introduced into the plant, as there is a positive correlation between high transcript levels and the triggering of co-suppression.

Another example of an RNA silencing method involves the use of antisense nucleic acid sequences. An “antisense” nucleic acid sequence comprises a nucleotide sequence that is complementary to a “sense” nucleic acid sequence encoding a protein, i.e. complementary to the coding strand of a double-stranded cDNA molecule or complementary to an mRNA transcript sequence. The antisense nucleic acid sequence is preferably complementary to the endogenous gene to be silenced. The complementarity may be located in the “coding region” and/or in the “non-coding region” of a gene. The term “coding region” refers to a region of the nucleotide sequence comprising codons that are translated into amino acid residues. The term “non-coding region” refers to 5′ and 3′ sequences that flank the coding region that are transcribed but not translated into amino acids (also referred to as 5′ and 3′ untranslated regions).

Antisense nucleic acid sequences can be designed according to the rules of Watson and Crick base pairing. The antisense nucleic acid sequence may be complementary to the entire nucleic acid sequence (in this case a stretch of substantially contiguous nucleotides derived from the gene of interest, or from any nucleic acid capable of encoding an orthologue, paralogue or homologue of the protein of interest), but may also be an oligonucleotide that is antisense to only a part of the nucleic acid sequence (including the mRNA 5′ and 3′ UTR). For example, the antisense oligonucleotide sequence may be complementary to the region surrounding the translation start site of an mRNA transcript encoding a polypeptide. The length of a suitable antisense oligonucleotide sequence is known in the art and may start from about 50, 45, 40, 35, 30, 25, 20, 15 or 10 nucleotides in length or less. An antisense nucleic acid sequence according to the invention may be constructed using chemical synthesis and enzymatic ligation reactions using methods known in the art. For example, an antisense nucleic acid sequence (e.g., an antisense oligonucleotide sequence) may be chemically synthesized using naturally occurring nucleotides or variously modified nucleotides designed to increase the biological stability of the molecules or to increase the physical stability of the duplex formed between the antisense and sense nucleic acid sequences, e.g., phosphorothioate derivatives and acridine substituted nucleotides may be used. Examples of modified nucleotides that may be used to generate the antisense nucleic acid sequences are well known in the art. Known nucleotide modifications include methylation, cyclization and ‘caps’ and substitution of one or more of the naturally occurring nucleotides with an analogue such as inosine. Other modifications of nucleotides are well known in the art.

The antisense nucleic acid sequence can be produced biologically using an expression vector into which a nucleic acid sequence has been subcloned in an antisense orientation (i.e., RNA transcribed from the inserted nucleic acid will be of an antisense orientation to a target nucleic acid of interest). Preferably, production of antisense nucleic acid sequences in plants occurs by means of a stably integrated nucleic acid construct comprising a promoter, an operably linked antisense oligonucleotide, and a terminator.

The nucleic acid molecules used for silencing in the methods of the invention (whether introduced into a plant or generated in situ) hybridize with or bind to mRNA transcripts and/or genomic DNA encoding a polypeptide to thereby inhibit expression of the protein, e.g., by inhibiting transcription and/or translation. The hybridization can be by conventional nucleotide complementarity to form a stable duplex, or, for example, in the case of an antisense nucleic acid sequence which binds to DNA duplexes, through specific interactions in the major groove of the double helix. Antisense nucleic acid sequences may be introduced into a plant by transformation or direct injection at a specific tissue site. Alternatively, antisense nucleic acid sequences can be modified to target selected cells and then administered systemically. For example, for systemic administration, antisense nucleic acid sequences can be modified such that they specifically bind to receptors or antigens expressed on a selected cell surface, e.g., by linking the antisense nucleic acid sequence to peptides or antibodies which bind to cell surface receptors or antigens. The antisense nucleic acid sequences can also be delivered to cells using the vectors described herein.

According to a further aspect, the antisense nucleic acid sequence is an a-anomeric nucleic acid sequence. An a-anomeric nucleic acid sequence forms specific double-stranded hybrids with complementary RNA in which, contrary to the usual b-units, the strands run parallel to each other (Gaultier et al. (1987) Nucl Ac Res 15: 6625-6641). The antisense nucleic acid sequence may also comprise a 2′-o-methylribonucleotide (Inoue et al. (1987) Nucl Ac Res 15, 6131-6148) or a chimeric RNA-DNA analogue (Inoue et al. (1987) FEBS Lett. 215, 327-330).

The reduction or substantial elimination of endogenous gene expression may also be performed using ribozymes. Ribozymes are catalytic RNA molecules with ribonuclease activity that are capable of cleaving a single-stranded nucleic acid sequence, such as an mRNA, to which they have a complementary region. Thus, ribozymes (e.g., hammerhead ribozymes (described in Haselhoff and Gerlach (1988) Nature 334, 585-591) can be used to catalytically cleave mRNA transcripts encoding a polypeptide, thereby substantially reducing the number of mRNA transcripts to be translated into a polypeptide. A ribozyme having specificity for a nucleic acid sequence can be designed (see for example: Cech et al. U.S. Pat. No. 4,987,071; and Cech et al. U.S. Pat. No. 5,116,742). Alternatively, mRNA transcripts corresponding to a nucleic acid sequence can be used to select a catalytic RNA having a specific ribonuclease activity from a pool of RNA molecules (Bartel and Szostak (1993) Science 261, 1411-1418). The use of ribozymes for gene silencing in plants is known in the art (e.g., Atkins et al. (1994) WO 94/00012; Lenne et al. (1995) WO 95/03404; Lutziger et al. (2000) WO 00/00619; Prinsen et al. (1997) WO 97/13865 and Scott et al. (1997) WO 97/38116).

Gene silencing may also be achieved by insertion mutagenesis (for example, T-DNA insertion or transposon insertion) or by strategies as described by, among others, Angell and Baulcombe ((1999) Plant J 20(3): 357-62), (Amplicon VIGS WO 98/36083), or Baulcombe (WO 99/15682).

Gene silencing may also occur if there is a mutation on an endogenous gene and/or a mutation on an isolated gene/nucleic acid subsequently introduced into a plant. The reduction or substantial elimination may be caused by a non-functional polypeptide. For example, the polypeptide may bind to various interacting proteins; one or more mutation(s) and/or truncation(s) may therefore provide for a polypeptide that is still able to bind interacting proteins (such as receptor proteins) but that cannot exhibit its normal function (such as signalling ligand).

A further approach to gene silencing is by targeting nucleic acid sequences complementary to the regulatory region of the gene (e.g., the promoter and/or enhancers) to form triple helical structures that prevent transcription of the gene in target cells. See Helene, C., Anticancer Drug Res. 6, 569-84, 1991; Helene et al., Ann. N.Y. Acad. Sci. 660, 27-36 1992; and Maher, L. J. Bioassays 14, 807-15, 1992.

Other methods, such as the use of antibodies directed to an endogenous polypeptide for inhibiting its function in planta, or interference in the signalling pathway in which a polypeptide is involved, will be well known to the skilled man. In particular, it can be envisaged that manmade molecules may be useful for inhibiting the biological function of a target polypeptide, or for interfering with the signalling pathway in which the target polypeptide is involved.

Alternatively, a screening program may be set up to identify in a plant population natural variants of a gene, which variants encode polypeptides with reduced activity. Such natural variants may also be used for example, to perform homologous recombination.

Artificial and/or natural microRNAs (miRNAs) may be used to knock out gene expression and/or mRNA translation. Endogenous miRNAs are single stranded small RNAs of typically 19-24 nucleotides long. They function primarily to regulate gene expression and/or mRNA translation. Most plant microRNAs (miRNAs) have perfect or near-perfect complementarity with their target sequences. However, there are natural targets with up to five mismatches. They are processed from longer non-coding RNAs with characteristic fold-back structures by double-strand specific RNases of the Dicer family. Upon processing, they are incorporated in the RNA-induced silencing complex (RISC) by binding to its main component, an Argonaute protein. MiRNAs serve as the specificity components of RISC, since they base-pair to target nucleic acids, mostly mRNAs, in the cytoplasm. Subsequent regulatory events include target mRNA cleavage and destruction and/or translational inhibition. Effects of miRNA overexpression are thus often reflected in decreased mRNA levels of target genes.

Artificial microRNAs (amiRNAs), which are typically 21 nucleotides in length, can be genetically engineered specifically to negatively regulate gene expression of single or multiple genes of interest. Determinants of plant microRNA target selection are well known in the art. Empirical parameters for target recognition have been defined and can be used to aid in the design of specific amiRNAs, (Schwab et al., Dev. Cell 8, 517-527, 2005). Convenient tools for design and generation of amiRNAs and their precursors are also available to the public (Schwab et al., Plant Cell 18, 1121-1133, 2006).

For optimal performance, the gene silencing techniques used for reducing expression in a plant of an endogenous gene requires the use of nucleic acid sequences from monocotyledonous plants for transformation of monocotyledonous plants, and from dicotyledonous plants for transformation of dicotyledonous plants. Preferably, a nucleic acid sequence from any given plant species is introduced into that same species. For example, a nucleic acid sequence from rice is transformed into a rice plant. However, it is not an absolute requirement that the nucleic acid sequence to be introduced originates from the same plant species as the plant in which it will be introduced. It is sufficient that there is substantial homology between the endogenous target gene and the nucleic acid to be introduced.

Described above are examples of various methods for the reduction or substantial elimination of expression in a plant of an endogenous gene. A person skilled in the art would readily be able to adapt the aforementioned methods for silencing so as to achieve reduction of expression of an endogenous gene in a whole plant or in parts thereof through the use of an appropriate promoter, for example.

### Transformation

The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al., (1986) Mol. Gen. Genet. 202: 179-185); DNA or RNA-coated particle bombardment (Klein T M et al., (1987) Nature 327: 70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-743). Methods for Agrobacterium-mediated transformation of rice include well known methods for rice transformation, such as those described in any of the following: European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Höfgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic [Feldman, K A and Marks M D (1987). Mol Gen Genet. 208:1-9; Feldmann K (1992). In: C Koncz, N-H Chua and J Shell, eds, Methods in Arabidopsis Research. Word Scientific, Singapore, pp. 274-289]. Alternative methods are based on the repeated removal of the inflorescences and incubation of the excision site in the center of the rosette with transformed agrobacteria, whereby transformed seeds can likewise be obtained at a later point in time (Chang (1994). Plant J. 5: 551-558; Katavic (1994). Mol Gen Genet, 245: 363-370). However, an especially effective method is the vacuum infiltration method with its modifications such as the “floral dip” method. In the case of vacuum infiltration of Arabidopsis, intact plants under reduced pressure are treated with an agrobacterial suspension [Bechthold, N (1993). C R Acad Sci Paris Life Sci, 316: 1194-1199], while in the case of the “floral dip” method the developing floral tissue is incubated briefly with a surfactant-treated agrobacterial suspension [Clough, S J and Bent A F (1998) The Plant J. 16, 735-743]. A certain proportion of transgenic seeds are harvested in both cases, and these seeds can be distinguished from non-transgenic seeds by growing under the above-described selective conditions. In addition the stable transformation of plastids is of advantages because plastids are inherited maternally is most crops reducing or eliminating the risk of transgene flow through pollen. The transformation of the chloroplast genome is generally achieved by a process which has been schematically displayed in Klaus et al., 2004 [Nature Biotechnology 22 (2), 225-229]. Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview is given in Bock (2001) Transgenic plastids in basic research and plant biotechnology. J Mol. Biol. 2001 Sep. 21; 312 (3):425-38 or Maliga, P (2003) Progress towards commercialization of plastid transformation technology. Trends Biotechnol. 21, 20-28. Further biotechnological progress has recently been reported in form of marker free plastid transformants, which can be produced by a transient co-integrated maker gene (Klaus et al., 2004, Nature Biotechnology 22(2), 225-229).

The genetically modified plant cells can be regenerated via all methods with which the skilled worker is familiar. Suitable methods can be found in the abovementioned publications by S. D. Kung and R. Wu, Potrykus or Hofgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

Throughout this application a plant, plant part, seed or plant cell transformed with—or interchangeably transformed by—a construct or transformed with or by a nucleic acid is to be understood as meaning a plant, plant part, seed or plant cell that carries said construct or said nucleic acid as a transgene due the result of an introduction of said construct or said nucleic acid by biotechnological means. The plant, plant part, seed or plant cell therefore comprises said recombinant construct or said recombinant nucleic acid. Any plant, plant part, seed or plant cell that no longer contains said recombinant construct or said recombinant nucleic acid after introduction in the past, is termed null-segregant, nullizygote or null control, but is not considered a plant, plant part, seed or plant cell transformed with said construct or with said nucleic acid within the meaning of this application.

### T-DNA Activation Tagging

T-DNA activation tagging (Hayashi et al. Science (1992) 1350-1353), involves insertion of T-DNA, usually containing a promoter (may also be a translation enhancer or an intron), in the genomic region of the gene of interest or 10 kb up- or downstream of the coding region of a gene in a configuration such that the promoter directs expression of the targeted gene. Typically, regulation of expression of the targeted gene by its natural promoter is disrupted and the gene falls under the control of the newly introduced promoter. The promoter is typically embedded in a T-DNA. This T-DNA is randomly inserted into the plant genome, for example, through Agrobacterium infection and leads to modified expression of genes near the inserted T-DNA. The resulting transgenic plants show dominant phenotypes due to modified expression of genes close to the introduced promoter.

### Tilling

The term “TILLING” is an abbreviation of “Targeted Induced Local Lesions In Genomes” and refers to a mutagenesis technology useful to generate and/or identify nucleic acids encoding proteins with modified expression and/or activity. TILLING also allows selection of plants carrying such mutant variants. These mutant variants may exhibit modified expression, either in strength or in location or in timing (if the mutations affect the promoter for example). These mutant variants may exhibit higher activity than that exhibited by the gene in its natural form. TILLING combines high-density mutagenesis with high-throughput screening methods. The steps typically followed in TILLING are: (a) EMS mutagenesis (Redei G P and Koncz C (1992) In Methods in Arabidopsis Research, Koncz C, Chua N H, Schell J, eds. Singapore, World Scientific Publishing Co, pp. 16-82; Feldmann et al., (1994) In Meyerowitz E M, Somerville C R, eds, Arabidopsis. Cold Spring Harbor Laboratory Press, Cold Spring Harbor, N.Y., pp 137-172; Lightner J and Caspar T (1998) In J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum et al., (2000) Nat Biotechnol 18: 455-457; reviewed by Stemple (2004) Nat Rev Genet. 5(2): 145-50).

### Homologous Recombination

Homologous recombination allows introduction in a genome of a selected nucleic acid at a defined selected position. Homologous recombination is a standard technology used routinely in biological sciences for lower organisms such as yeast or the moss Physcomitrella. Methods for performing homologous recombination in plants have been described not only for model plants (Offring a et al. (1990) EMBO J. 9(10): 3077-84) but also for crop plants, for example rice (Terada et al. (2002) Nat Biotech 20(10): 1030-4; lida and Terada (2004) Curr Opin Biotech 15(2): 132-8), and approaches exist that are generally applicable regardless of the target organism (Miller et al, Nature Biotechnol. 25, 778-785, 2007).

### Yield Related Traits

Yield related traits are traits or features which are related to plant yield. Yield-related traits may comprise one or more of the following non-limitative list of features: early flowering time, yield, biomass, seed yield, early vigour, greenness index, increased growth rate, improved agronomic traits, such as e.g. increased tolerance to submergence (which leads to increased yield in rice), improved Water Use Efficiency (WUE), improved Nitrogen Use Efficiency (NUE), etc.

### Yield

The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square meters.

The terms “yield” of a plant and “plant yield” are used interchangeably herein and are meant to refer to vegetative biomass such as root and/or shoot biomass, to reproductive organs, and/or to propagules such as seeds of that plant.

Flowers in maize are unisexual; male inflorescences (tassels) originate from the apical stem and female inflorescences (ears) arise from axillary bud apices. The female inflorescence produces pairs of spikelets on the surface of a central axis (cob). Each of the female spikelets encloses two fertile florets, one of them will usually mature into a maize kernel once fertilized. Hence a yield increase in maize may be manifested as one or more of the following: increase in the number of plants established per square meter, an increase in the number of ears per plant, an increase in the number of rows, number of kernels per row, kernel weight, thousand kernel weight, ear length/diameter, increase in the seed filling rate, which is the number of filled florets (i.e. florets containing seed) divided by the total number of florets and multiplied by 100), among others.

Inflorescences in rice plants are named panicles. The panicle bears spikelets, which are the basic units of the panicles, and which consist of a pedicel and a floret. The floret is borne on the pedicel and includes a flower that is covered by two protective glumes: a larger glume (the lemma) and a shorter glume (the palea). Hence, taking rice as an example, a yield increase may manifest itself as an increase in one or more of the following: number of plants per square meter, number of panicles per plant, panicle length, number of spikelets per panicle, number of flowers (or florets) per panicle; an increase in the seed filling rate which is the number of filled florets (i.e. florets containing seeds) divided by the total number of florets and multiplied by 100; an increase in thousand kernel weight, among others.

### Early Flowering Time

Plants having an “early flowering time” as used herein are plants which start to flower earlier than control plants. Hence this term refers to plants that show an earlier start of flowering. Flowering time of plants can be assessed by counting the number of days (“time to flower”) between sowing and the emergence of a first inflorescence. The “flowering time” of a plant can for instance be determined using the method as described in WO 2007/093444.

### Early Vigour

“Early vigour” refers to active healthy well-balanced growth especially during early stages of plant growth, and may result from increased plant fitness due to, for example, the plants being better adapted to their environment (i.e. optimizing the use of energy resources and partitioning between shoot and root). Plants having early vigour also show increased seedling survival and a better establishment of the crop, which often results in highly uniform fields (with the crop growing in uniform manner, i.e. with the majority of plants reaching the various stages of development at substantially the same time), and often better and higher yield. Therefore, early vigour may be determined by measuring various factors, such as thousand kernel weight, percentage germination, percentage emergence, seedling growth, seedling height, root length, root and shoot biomass and many more.

### Increased Growth Rate

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as speed of germination, early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per square meter (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

### Stress Resistance

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35%, 30% or 25%, more preferably less than 20% or 15% in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. “Mild stresses” are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures.

“Biotic stresses” are typically those stresses caused by pathogens, such as bacteria, viruses, fungi, nematodes and insects.

The “abiotic stress” may be an osmotic stress caused by a water stress, e.g. due to drought, salt stress, or freezing stress. Abiotic stress may also be an oxidative stress or a cold stress. “Freezing stress” is intended to refer to stress due to freezing temperatures, i.e. temperatures at which available water molecules freeze and turn into ice. “Cold stress”, also called “chilling stress”, is intended to refer to cold temperatures, e.g. temperatures below 10°, or preferably below 5° C., but at which water molecules do not freeze. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location. Plants with optimal growth conditions, (grown under non-stress conditions) typically yield in increasing order of preference at least 97%, 95%, 92%, 90%, 87%, 85%, 83%, 80%, 77% or 75% of the average production of such plant in a given environment. Average production may be calculated on harvest and/or season basis. Persons skilled in the art are aware of average yield productions of a crop.

In particular, the methods of the present invention may be performed under non-stress conditions. In an example, the methods of the present invention may be performed under non-stress conditions such as mild drought to give plants having increased yield relative to control plants.

In another embodiment, the methods of the present invention may be performed under stress conditions, preferably under abiotic stress conditions.

In an example, the methods of the present invention may be performed under abiotic environmental stress conditions such as drought to give plants having increased yield relative to control plants.

In another example, the methods of the present invention may be performed under abiotic environmental stress conditions such as nutrient deficiency to give plants having increased yield relative to control plants.

Nutrient deficiency may result from a lack of nutrients such as nitrogen, phosphates and other phosphorous-containing compounds, potassium, calcium, magnesium, manganese, iron and boron, amongst others.

In yet another example, the methods of the present invention may be performed under abiotic environmental stress conditions such as salt stress to give plants having increased yield relative to control plants. The term salt stress is not restricted to common salt (NaCl), but may be any one or more of: NaCl, KCl, LiCl, MgCl₂, CaCl₂, amongst others.

In yet another example, the methods of the present invention may be performed under abiotic environmental stress conditions such as cold stress or freezing stress to give plants having increased yield relative to control plants.

### Increase/Improve/Enhance

The terms “increase”, “improve” or “enhance” are interchangeable and shall mean in the sense of the application at least a 3%, 4%, 5%, 6%, 7%, 8%, 9% or 10%, preferably at least 15% or 20%, more preferably 25%, 30%, 35% or 40% more yield and/or growth in comparison to control plants as defined herein.

The terms“relative to control plants” and “compared to control plants” are interchangeable and shall mean in the sense of the application that the yield-related parameters and/or fine chemical of the altered plant are compared with the corresponding values of the control plant grown under conditions as similar as possible.

### Seed Yield

Increased seed yield may manifest itself as one or more of the following:
a) an increase in seed biomass (total seed weight) which may be on an individual seed basis and/or per plant and/or per square meter;
b) increased number of flowers per plant;
c) increased number of seeds;
d) increased seed filling rate (which is expressed as the ratio between the number of filled florets divided by the total number of florets);
e) increased harvest index, which is expressed as a ratio of the yield of harvestable parts, such as seeds, divided by the biomass of aboveground plant parts; and
f) increased thousand kernel weight (TKW), which is extrapolated from the number of seeds counted and their total weight. An increased TKW may result from an increased seed size and/or seed weight, and may also result from an increase in embryo and/or endosperm size.

The terms “filled florets” and “filled seeds” may be considered synonyms.

An increase in seed yield may also be manifested as an increase in seed size and/or seed volume. Furthermore, an increase in seed yield may also manifest itself as an increase in seed area and/or seed length and/or seed width and/or seed perimeter.

### Greenness Index

The “greenness index” as used herein is calculated from digital images of plants. For each pixel belonging to the plant object on the image, the ratio of the green value versus the red value (in the RGB model for encoding color) is calculated. The greenness index is expressed as the percentage of pixels for which the green-to-red ratio exceeds a given threshold. Under normal growth conditions, under salt stress growth conditions, and under reduced nutrient availability growth conditions, the greenness index of plants is measured in the last imaging before flowering. In contrast, under drought stress growth conditions, the greenness index of plants is measured in the first imaging after drought.

### Biomass

The term “biomass” as used herein is intended to refer to the total weight of a plant. Within the definition of biomass, a distinction may be made between the biomass of one or more parts of a plant, which may include any one or more of the following:
aboveground parts such as but not limited to shoot biomass, seed biomass, leaf biomass, etc.;
aboveground harvestable parts such as but not limited to shoot biomass, seed biomass, leaf biomass, etc.;
parts below ground, such as but not limited to root biomass, tubers, bulbs, etc.;
harvestable parts below ground, such as but not limited to root biomass, tubers, bulbs, etc.;
harvestable parts partly inserted in or in contact with the ground such as but not limited to beets and other hypocotyl areas of a plant, rhizomes, stolons or creeping rootstalks;
vegetative biomass such as root biomass, shoot biomass, etc.;
reproductive organs; and propagules such as seed.

### Marker Assisted Breeding

Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

### Use as Probes in (Gene Mapping)

Use of nucleic acids encoding the protein of interest for genetically and physically mapping the genes requires only a nucleic acid sequence of at least 15 nucleotides in length. These nucleic acids may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the nucleic acids encoding the protein of interest. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the nucleic acid encoding the protein of interest in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med. 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

### Plant

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocaffis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

With respect to the sequences of the invention, a nucleic acid or a polypeptide sequence of plant origin has the characteristic of a codon usage optimised for expression in plants, and of the use of amino acids and regulatory sites common in plants, respectively. The plant of origin may be any plant, but preferably those plants as described in the previous paragraph.

### Control Plant(s)

The choice of suitable control plants is a routine part of an experimental setup and may include corresponding wild type plants or corresponding plants without the gene of interest. The control plant is typically of the same plant species or even of the same variety as the plant to be assessed. The control plant may also be a nullizygote of the plant to be assessed. Nullizygotes (also called null control plants) are individuals missing the transgene by segregation. Further, a control plant has been grown under equal growing conditions to the growing conditions of the plants of the invention. Typically the control plant is grown under equal growing conditions and hence in the vicinity of the plants of the invention and at the same time. A “control plant” as used herein refers not only to whole plants, but also to plant parts, including seeds and seed parts.

### DETAILED DESCRIPTION OF THE INVENTION

Surprisingly, it has now been found that modulating expression in a plant of a nucleic acid encoding a POI polypeptide gives plants having enhanced yield-related traits relative to control plants.

According to a first embodiment, the present invention provides a method for enhancing yield-related traits in plants relative to control plants, comprising modulating expression in a plant of a nucleic acid encoding a POI polypeptide and optionally selecting for plants having enhanced yield-related traits. According to another embodiment, the present invention provides a method for producing plants having enhancing yield-related traits relative to control plants, wherein said method comprises the steps of modulating expression in said plant of a nucleic acid encoding a POI polypeptide as described herein and optionally selecting for plants having enhanced yield-related traits.

A preferred method for modulating (preferably, increasing) expression of a nucleic acid encoding a POI polypeptide is by introducing and expressing in a plant a nucleic acid encoding a POI polypeptide.

Any reference hereinafter to a “protein useful in the methods of the invention” is taken to mean a POI polypeptide as defined herein. Any reference hereinafter to a “nucleic acid useful in the methods of the invention” is taken to mean a nucleic acid capable of encoding such a POI polypeptide. The nucleic acid to be introduced into a plant (and therefore useful in performing the methods of the invention) is any nucleic acid encoding the type of protein which will now be described, hereafter also named “POI nucleic acid” or “POI gene”.

A “POI polypeptide” as defined herein refers to any DnaJ-like chaperone polypeptide, preferably to any sequence provided by SEQ ID NO in column 5 or 7 of table II or encoded by a polynucleotide as represented by the SEQ ID NOs in column 5 and 7 of table I, or homologs thereof.

In one embodiment the DnaJ-like chaperone polypeptide useful in the processes of the invention comprises the three PFAM domains DnaJ (PF00226), DnaJ_C (PF01556) (DnaJ_C=DnaJ C terminal domain) and DnaJ_CXXCXGXG (PF00684) DnaJ central domain (according to the PFAM database release 25.0 (released March 2011) of the Welcome Trust SANGER Institute, Hinxton, England, UK (http://pfam.sanger.ac.uk/).

In another embodiment the DnaJ-like chaperone polypeptide comprises one or more of the consensus patterns shown in SEQ ID NOs: 45, 46 and 47.

In a preferred embodiment the DnaJ-like chaperone polypeptide comprises the amino acids at position 6 to 67, 143 to 208 and 265 to 348 of YNL064C (SEQ ID NO: 2).

The term “POI” or “POI polypeptide” as used herein also intends to include homologues as defined hereunder of “POI polypeptide”, i.e. DnaJ-like chaperone polypeptides as defined herein and homologues as defined hereunder.

Additionally or alternatively, the homologue of a POI protein, i.e. DnaJ-like chaperone polypeptide has in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 81%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 2 or 42, preferably by SEQ ID NO: 2, provided that the homologous protein comprises any one or more of the conserved PFAM domains as outlined above, preferably at least and more preferably all three of the PFAM domains as outlined above. The overall sequence identity is determined using a global alignment algorithm, such as the Needleman Wunsch algorithm in the program GAP (GCG Wisconsin Package, Accelrys), preferably with default parameters and preferably with sequences of mature proteins (i.e. without taking into account secretion signals or transit peptides).

In one embodiment the sequence identity level is determined by comparison of the polypeptide sequences over the entire length of the sequence of SEQ ID NO: 2 or 42, preferably SEQ ID NO: 2.

In another embodiment the sequence identity level of a nucleic acid sequence is determined by comparison of the nucleic acid sequence over the entire length of the coding sequence of the sequence of SEQ ID NO: 1 or 41, preferably SEQ ID NO:1.

In another embodiment a method is provided wherein said DnaJ-like chaperone polypeptide comprises a sequence part with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to any one of the consensus patterns represented by the sequence of SEQ ID NO:45, 46 or 47. In a preferred embodiment the DnaJ-like chaperone polypeptide comprises sequence parts with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to all three of the consensus patterns represented by the sequence of SEQ ID NO:45, 46 or 47.

In another embodiment a method is provided wherein said DnaJ-like chaperone polypeptide comprises a conserved domain (or motif) with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the conserved domain starting with amino acid 6 up to amino acid 67 and/or to the conserved domain starting with amino acid 143 up to amino acid 208 and/or to the conserved domain starting with amino acid 265 up to amino acid 348 in SEQ ID NO:2.

The terms “domain”, “signature” and “motif” are defined in the “definitions” section herein.

In one embodiment the DnaJ-like chaperone polypeptides employed in the methods, constructs, plants, harvestable parts and products of the invention are DnaJ-like chaperones but excluding the DnaJ-like chaperones of the sequences disclosed in SEQ ID NO: 42

Preferably, the polypeptide sequence which when used in the construction of a phylogenetic tree clusters with the group of DnaJ-like chaperone polypeptides comprising the amino acid sequence represented by SEQ ID NO: 2 and/or 42, preferably 2 rather than with any other group. In another embodiment the polypeptides of the invention when used in the construction of a phylogenetic tree cluster not more than 4, 3, or 2 hierarchical branch points away from the amino acid sequence of SEQ ID NO:2 and/or 42, preferably 2.

Furthermore, DnaJ-like chaperone polypeptides (at least in their native form) typically have chaperone activity. Tools and techniques for measuring chaperone activity are well known in the art.

In addition, DnaJ-like chaperone polypeptides, when expressed in plants such as Arabidopsis according to the methods of the present invention as outlined in Examples 8 and 9, give plants having increased yield related traits, in particular under conditions of stress, more preferably under conditions of water limitation, most preferably under conditions of drought stress, and/or result in the increased production of a fine chemical as listed in table FC.

A further embodiment of the present invention relates to methods for increasing the content of any one or more fine chemical listed in table FC in plants compared to control plants and for simultaneously enhancing yield-related traits in plants under environmental stress conditions and/or non-stress conditions in plants relative to control plants, comprising modulating expression in a plant of nucleic acids encoding a DnaJ like chaperone as defined above. In one embodiment the methods of the invention are methods to for increasing the content of any one or more fine chemical listed in table FC in plants compared to control plants and for enhancing at the same time yield-related traits in plants under abiotic environmental stress conditions, preferably under conditions of limited water availability, more preferably under conditions of drought, in plants relative to control plants, comprising modulating expression in a plant of nucleic acids encoding a DnaJ like chaperone as defined above. In another embodiment the methods of the invention are for increasing the content of any one or more fine chemicals listed in table FC in plants compared to control plants and for enhancing at the same time yield-related traits in plants under non-stress conditions in plants relative to control plants, comprising modulating expression in a plant of nucleic acids encoding a DnaJ like chaperone as defined above. In another embodiment the methods of the invention modulate the expression of said nucleic acids encoding a DnaJ like chaperone as defined above by introducing and expressing said nucleic acids, preferably by introducing and expressing said nucleic acids by biotechnological means as recombinant nucleic acids, preferably by stable integration into the genome of the plant.

The present invention is illustrated by transforming plants with the nucleic acid sequence represented by SEQ ID NO: 1, encoding the polypeptide sequence of SEQ ID NO: 2. However, performance of the invention is not restricted to these sequences; the methods of the invention may advantageously be performed using any DnaJ-like chaperone-encoding nucleic acid or DnaJ-like chaperone polypeptide as defined herein.

Examples of nucleic acids encoding DnaJ-like chaperone polypeptides are given in Table II. Such nucleic acids are useful in performing the methods of the invention. The amino acid sequences given in table II of the Examples section are example sequences of orthologues and paralogues of the DnaJ-like chaperone polypeptide represented by SEQ ID NO: 2 or 42, preferably by SEQ ID NO: 2, the terms “orthologues” and “paralogues” being as defined herein. Further orthologues and paralogues may readily be identified by performing a so-called reciprocal blast search as described in the definitions section; where the query sequence is SEQ ID NO: 1 or SEQ ID NO: 2, the second BLAST (back-BLAST) would be against Saccharomyces cerevisiae sequences.

According to a further embodiment of the present invention, there are therefore provided an isolated nucleic acid molecule useful in the methods, processes, uses selected from:
(i) a nucleic acid represented by SEQ ID NO: 1 3, 5, 7, 9, 11, 13, 15, 17, 19, 21, 23, 25, 27, 29, 31, 33, 35, 37, 39 or 41;
(ii) the complement of a nucleic acid represented by SEQ ID NO: 1 3, 5, 7, 9, 11, 13, 15, 17, 19, 21, 23, 25, 27, 29, 31, 33, 35, 37, 39 or 41;
(iii) a nucleic acid encoding a DnaJ-like chaperone polypeptide having in increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence represented by SEQ ID NO: 2, 4, 6, 8, 10, 12, 14, 16, 18, 20, 22, 24, 26, 28, 30, 32, 34, 36, 38, 40 or 42 and additionally comprising one or more domains having in increasing order of preference at least 50%, 55%, 60%, 65%, 70%, 75%, 80%, 85%, 90%, 95%, 96%, 97%, 98%, 99% or 100% sequence identity to any one or more of the PFAM domains PF00226, PF01556 and PF00684, preferably to the conserved domain starting with amino acid 6 up to amino acid 67 and/or to the conserved domain starting with amino acid 143 up to amino acid 208 and/or to the conserved domain starting with amino acid 265 up to amino acid 348 in SEQ ID NO:2, and further preferably conferring enhanced yield-related traits relative to control plants under stress conditions, preferably under abiotic environmental stress conditions as defined herein, and/or increased fine chemical content of one or more fine chemicals as listed in table FC.
(iv) a nucleic acid encoding a DnaJ-like chaperone polypeptide comprising one or more, preferably to all three of the consensus patterns of SEQ ID NO: 45, 46 and 47 and further preferably conferring enhanced yield-related traits relative to control plants under stress conditions, preferably under abiotic environmental stress conditions as defined herein, and/or increased fine chemical content of one or more fine chemicals as listed in table FC;
(v) a nucleic acid molecule which hybridizes with a nucleic acid molecule of (i) to (iii) under high stringency hybridization conditions and preferably confers enhanced yield-related traits relative to control plants under stress conditions, preferably under abiotic environmental stress conditions as defined herein, and/or increased fine chemical content of one or more fine chemicals as listed in table FC.

According to a further embodiment of the present invention, there is also provided an isolated polypeptide selected from:
(i) an amino acid sequence represented by SEQ ID NO: Y;
(ii) an amino acid sequence having, in increasing order of preference, at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence represented by SEQ ID NO: 2, 4, 6, 8, 10, 12, 14, 16, 18, 20, 22, 24, 26, 28, 30, 32, 34, 36, 38, 40 or 42, and additionally comprising one or more domains having in increasing order of preference at least 50%, 55%, 60%, 65%, 70%, 75%, 80%, 85%, 90%, 95%, 96%, 97%, 98%, 99% or more sequence identity to any one or more of the PFAM domains PF00226, PF01556 and PF00684, preferably to the conserved domain starting with amino acid 6 up to amino acid 67 and/or to the conserved domain starting with amino acid 143 up to amino acid 208 and/or to the conserved domain starting with amino acid 265 up to amino acid 348 in SEQ ID NO:2, and further preferably conferring enhanced yield-related traits relative to control plants under stress conditions, preferably under abiotic environmental stress conditions as defined herein, and/or non-stress conditions, and/or increased fine chemical content of one or more fine chemicals as listed in table FC;
(iii) a nucleic acid encoding a DnaJ-like chaperone polypeptide comprising one or more, preferably to all three of the consensus patterns of SEQ ID NO: 45, 46 and 47 and further preferably conferring enhanced yield-related traits relative to control plants under stress conditions, preferably under abiotic environmental stress conditions as defined herein, and/or non-stress conditions, and/or increased fine chemical content of one or more fine chemicals as listed in table FC;
(iv) derivatives of any of the amino acid sequences given in (i) or (ii) above.

Nucleic acid variants may also be useful in practising the methods of the invention. Examples of such variants include nucleic acids encoding homologues and derivatives of any one of the amino acid sequences given in table II of the Examples section, the terms “homologue” and “derivative” being as defined herein. Also useful in the methods of the invention are nucleic acids encoding homologues and derivatives of orthologues or paralogues of any one of the amino acid sequences given in table II of the Examples section. Homologues and derivatives useful in the methods of the present invention have substantially the same biological and functional activity as the unmodified protein from which they are derived. Further variants useful in practising the methods of the invention are variants in which codon usage is optimised or in which miRNA target sites are removed.

Further nucleic acid variants useful in practising the methods of the invention include portions of nucleic acids encoding DnaJ-like chaperone polypeptides, nucleic acids hybridising to nucleic acids encoding DnaJ-like chaperone polypeptides, splice variants of nucleic acids encoding DnaJ-like chaperone polypeptides, allelic variants of nucleic acids encoding DnaJ-like chaperone polypeptides and variants of nucleic acids encoding DnaJ-like chaperone polypeptides obtained by gene shuffling. The terms hybridising sequence, splice variant, allelic variant and gene shuffling are as described herein.

In one embodiment of the present invention the function of the nucleic acid sequences of the invention is to confer information for a protein that increases yield or yield related traits, when a nucleic acid sequence of the invention is transcribed and translated in a living plant cell.

Nucleic acids encoding DnaJ-like chaperone polypeptides need not be full-length nucleic acids, since performance of the methods of the invention does not rely on the use of full-length nucleic acid sequences. According to the present invention, there is provided a method for enhancing yield-related traits in plants, comprising introducing and expressing in a plant a portion of any one of the nucleic acid sequences given in Table A of the Examples section, or a portion of a nucleic acid encoding an orthologue, paralogue or homologue of any of the amino acid sequences given in table II of the Examples section.

A portion of a nucleic acid may be prepared, for example, by making one or more deletions to the nucleic acid. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Portions useful in the methods of the invention, encode a DnaJ-like chaperone polypeptide as defined herein, and have substantially the same biological activity as the amino acid sequences given in table II of the Examples section. Preferably, the portion is a portion of any one of the nucleic acids given in Table I of the Examples section, or is a portion of a nucleic acid encoding an orthologue or paralogue of any one of the amino acid sequences given in table II of the Examples section. Preferably the portion is at least 500, 550, 600, 650, 700, 750, 800, 850, 900, 950, 1000, 1050, 1100, 1150, 1200, 1250, 1300 consecutive nucleotides in length, the consecutive nucleotides being of any one of the nucleic acid sequences given in Table I of the Examples section, or of a nucleic acid encoding an orthologue or paralogue of any one of the amino acid sequences given in table II of the Examples section. Most preferably the portion is a portion of the nucleic acid of SEQ ID NO: 1. Preferably, the portion encodes a fragment of an amino acid sequence which, when used in the construction of a phylogenetic tree, clusters with the group of DnaJ-like chaperone polypeptides comprising the amino acid sequence represented by SEQ ID NO: 2 or 42, preferably by SEQ ID NO: 2 rather than with any other group, and/or comprises .the PFAM domains PF00226, PF01556 and PF00684, or one or more, preferably all three of the consensus pattern as provided in SEQ ID NO: 45, 46 and 47 preferably it comprises the conserved domain starting with amino acid 6 up to amino acid 67 and/or to the conserved domain starting with amino acid 143 up to amino acid 208 and/or to the conserved domain starting with amino acid 265 up to amino acid 348 in SEQ ID NO:2

Another nucleic acid variant useful in the methods of the invention is a nucleic acid capable of hybridising, under reduced stringency conditions, preferably under stringent conditions, with a nucleic acid encoding a DnaJ-like chaperone polypeptide as defined herein, or with a portion as defined herein.

According to the present invention, there is provided a method for enhancing yield-related traits in plants, comprising introducing and expressing in a plant a nucleic acid capable of hybridizing to any one of the nucleic acids given in Table I of the Examples section, or comprising introducing and expressing in a plant a nucleic acid capable of hybridising to a nucleic acid encoding an orthologue, paralogue or homologue of any of the nucleic acid sequences given in Table A of the Examples section.

Hybridising sequences useful in the methods of the invention encode a DnaJ-like chaperone polypeptide as defined herein, having substantially the same biological activity as the amino acid sequences given in table II of the Examples section. Preferably, the hybridising sequence is capable of hybridising to the complement of any one of the nucleic acids given in Table I of the Examples section, or to a portion of any of these sequences, a portion being as defined above, or the hybridising sequence is capable of hybridising to the complement of a nucleic acid encoding an orthologue or paralogue of any one of the amino acid sequences given in table II of the Examples section. Most preferably, the hybridising sequence is capable of hybridising to the complement of a nucleic acid as represented by SEQ ID NO: 1 or 41, preferably by SEQ ID NO: 1 or to a portion thereof.

Preferably, the hybridising sequence encodes a polypeptide with an amino acid sequence which, when full-length and used in the construction of a phylogenetic tree clusters with the group of DnaJ-like chaperone polypeptides comprising the amino acid sequence represented by SEQ ID NO: 2 or 42, preferably by SEQ ID NO: 2 rather than with any other group, and/or comprises .the PFAM domains PF00226, PF01556 and PF00684, or one or more, preferably all three of the consensus pattern as provided in SEQ ID NO: 45, 46 and 47 preferably it comprises the conserved domain starting with amino acid 6 up to amino acid 67 and/or to the conserved domain starting with amino acid 143 up to amino acid 208 and/or to the conserved domain starting with amino acid 265 up to amino acid 348 in SEQ ID NO:2

In one embodiment the hybridising sequence is capable of hybridising to the complement of a nucleic acid as represented by SEQ ID NO: 1 or 41, preferably by SEQ ID NO: 1 or to a portion thereof under conditions of medium or high stringency, preferably high stringency as defined above. In another embodiment the hybridising sequence is capable of hybridising to the complement of a nucleic acid as represented by SEQ ID NO: 1 or 41, preferably by SEQ ID NO: 1 under stringent conditions.

Another nucleic acid variant useful in the methods of the invention is a splice variant encoding a DnaJ-like chaperone polypeptide as defined hereinabove, a splice variant being as defined herein.

According to the present invention, there is provided a method for enhancing yield-related traits in plants, comprising introducing and expressing in a plant a splice variant of any one of the nucleic acid sequences given in Table A of the Examples section, or a splice variant of a nucleic acid encoding an orthologue, paralogue or homologue of any of the amino acid sequences given in table II of the Examples section.

Preferred splice variants are splice variants of a nucleic acid represented by SEQ ID NO: 1 or 41, preferably by SEQ ID NO: 1, or a splice variant of a nucleic acid encoding an orthologue or paralogue of SEQ ID NO: 2. Preferably, the amino acid sequence encoded by the splice variant, when used in the construction of a phylogenetic tree clusters with the group of DnaJ-like chaperone polypeptides comprising the amino acid sequence represented by SEQ ID NO: 2 or 42, preferably by SEQ ID NO: 2 rather than with any other group and/or comprises .the PFAM domains PF00226, PF01556 and PF00684, or one or more, preferably all three of the consensus pattern as provided in SEQ ID NO: 45, 46 and 47 preferably it comprises the conserved domain starting with amino acid 6 up to amino acid 67 and/or to the conserved domain starting with amino acid 143 up to amino acid 208 and/or to the conserved domain starting with amino acid 265 up to amino acid 348 in SEQ ID NO:2

Another nucleic acid variant useful in performing the methods of the invention is an allelic variant of a nucleic acid encoding a DnaJ-like chaperone polypeptide as defined hereinabove, an allelic variant being as defined herein.

According to the present invention, there is provided a method for enhancing yield-related traits in plants, comprising introducing and expressing in a plant an allelic variant of any one of the nucleic acids given in Table I of the Examples section, or comprising introducing and expressing in a plant an allelic variant of a nucleic acid encoding an orthologue, paralogue or homologue of any of the amino acid sequences given in table II of the Examples section.

The polypeptides encoded by allelic variants useful in the methods of the present invention have substantially the same biological activity as the DnaJ-like chaperone polypeptide of SEQ ID NO: 2 and any of the amino acids depicted in Table A of the Examples section. Allelic variants exist in nature, and encompassed within the methods of the present invention is the use of these natural alleles. Preferably, the allelic variant is an allelic variant of SEQ ID NO: 1 or an allelic variant of a nucleic acid encoding an orthologue or paralogue of SEQ ID NO: 2. Preferably, the amino acid sequence encoded by the allelic variant, when used in the construction of a phylogenetic tree clusters with the DnaJ-like chaperone polypeptides comprising the amino acid sequence represented by SEQ ID NO: 2 or 42, preferably by SEQ ID NO: 2 rather than with any other group and/or comprises .the PFAM domains PF00226, PF01556 and PF00684, or one or more, preferably all three of the consensus pattern as provided in SEQ ID NO: 45, 46 and 47 preferably it comprises the conserved domain starting with amino acid 6 up to amino acid 67 and/or to the conserved domain starting with amino acid 143 up to amino acid 208 and/or to the conserved domain starting with amino acid 265 up to amino acid 348 in SEQ ID NO:2

Gene shuffling or directed evolution may also be used to generate variants of nucleic acids encoding DnaJ-like chaperone polypeptides as defined above; the term “gene shuffling” being as defined herein.

According to the present invention, there is provided a method for enhancing yield-related traits in plants, comprising introducing and expressing in a plant a variant of any one of the nucleic acid sequences given in Table A of the Examples section, or comprising introducing and expressing in a plant a variant of a nucleic acid encoding an orthologue, paralogue or homologue of any of the amino acid sequences given in table II of the Examples section, which variant nucleic acid is obtained by gene shuffling.

Preferably, the amino acid sequence encoded by the variant nucleic acid obtained by gene shuffling, when used in the construction of a phylogenetic tree clusters with the group of DnaJ-like chaperone polypeptides comprising the amino acid sequence represented by SEQ ID NO: 2 or 42, preferably by SEQ ID NO: 2 rather than with any other group and/or comprises .the PFAM domains PF00226, PF01556 and PF00684, or one or more, preferably all three of the consensus pattern as provided in SEQ ID NO: 45, 46 and 47 preferably it comprises the conserved domain starting with amino acid 6 up to amino acid 67 and/or to the conserved domain starting with amino acid 143 up to amino acid 208 and/or to the conserved domain starting with amino acid 265 up to amino acid 348 in SEQ ID NO:2

Furthermore, nucleic acid variants may also be obtained by site-directed mutagenesis. Several methods are available to achieve site-directed mutagenesis, the most common being PCR based methods (Current Protocols in Molecular Biology. Wiley Eds.).

Nucleic acids encoding DnaJ-like chaperone polypeptides may be derived from any natural or artificial source. The nucleic acid may be modified from its native form in composition and/or genomic environment through deliberate human manipulation. Preferably the DnaJ-like chaperone polypeptide-encoding nucleic acid is from a yeast or a plant, further preferably from a monocotyledonous plant or a Saccharomyces yeast, more preferably the nucleic acid is from Oryza sativa or Saccharomyces cerevisiae, most preferably from Saccharomyces cerevisiae.

In another embodiment the present invention extends to recombinant chromosomal DNA comprising a nucleic acid sequence useful in the methods of the invention, wherein said nucleic acid is present in the chromosomal DNA as a result of recombinant methods, i.e. said nucleic acid is not in the chromosomal DNA in its native surrounding. Said recombinant chromosomal DNA may be a chromosome of native origin, with said nucleic acid inserted by recombinant means, or it may be a mini-chromosome or a non-native chromosomal structure, e.g. or an artificial chromosome. The nature of the chromosomal DNA may vary, as long it allows for stable passing on to successive generations of the recombinant nucleic acid useful in the methods of the invention, and allows for expression of said nucleic acid in a living plant cell resulting in increased yield or increased yield related traits of the plant cell or a plant comprising the plant cell.

In a further embodiment the recombinant chromosomal DNA of the invention is comprised in a plant cell.

Performance of the methods of the invention gives plants having enhanced yield-related traits under abiotic environmental stress conditions and/or non-stress conditions, and/or increased content of any one or more fine chemical listed in table FC relative to control plants. In particular performance of the methods of the invention gives plants having increased yield, especially increased seed yield and/or biomass relative to control plants, under abiotic environmental stress conditions and/or non-stress conditions, preferably under conditions of limited water availability, more preferably under conditions of drought, and/or increased content of any one or more fine chemical listed in table FC relative to control plants. The terms “yield” and “seed yield” and “biomass” are described in more detail in the “definitions” section herein.

Reference herein to enhanced yield-related traits is taken to mean an increase early vigour and/or in biomass (weight) of one or more parts of a plant, which may include (i) aboveground parts and preferably aboveground harvestable parts and/or (ii) parts below ground and preferably harvestable below ground. In particular, such harvestable parts are roots such as taproots, stems, beets, leaves, flowers or seeds, and performance of the methods of the invention results in plants having increased seed yield relative to the seed yield of control plants, and/or increased stem biomass relative to the stem biomass of control plants, and/or increased root biomass relative to the root biomass of control plants and/or increased beet biomass relative to the beet biomass of control plants. Moreover, it is particularly contemplated that the sugar content (in particular the sucrose content) in the stem (in particular of sugar cane plants) and/or in the root (in particular in sugar beets) is increased relative to the sugar content (in particular the sucrose content) in the stem and/or in the root of the control plant.

The present invention provides a method for increasing yield-related traits—yield, especially biomass and/or seed yield of plants, relative to control plants, under stress conditions, preferably under abiotic environmental stress conditions as defined herein, and/or non-stress conditions, preferably under conditions of limited water availability, more preferably under conditions of drought, and/or increased content of any one or more fine chemical listed in table FC relative to control plants; which method comprises modulating expression in a plant of a nucleic acid encoding a DnaJ-like chaperone polypeptide as defined herein.

According to a preferred feature of the present invention, performance of the methods of the invention gives plants having an increased growth rate under abiotic environmental stress conditions and/or non-stress conditions, preferably under conditions of limited water availability, more preferably under conditions of drought, and/or increased content of any one or more fine chemical listed in table FC; relative to control plants. Therefore, according to the present invention, there is provided a method for increasing the growth rate of plants, which method comprises modulating expression in a plant of a nucleic acid encoding a DnaJ-like chaperone polypeptide as defined herein.

Performance of the methods of the invention gives plants grown under abiotic environmental stress conditions and/or non-stress conditions, particularly under drought conditions increased yield relative to control plants grown under comparable conditions. Therefore, according to the present invention, there is provided a method for increasing yield in plants grown under abiotic environmental stress conditions and/or non-stress conditions, particularly mild drought conditions, which method comprises modulating expression in a plant of a nucleic acid encoding a DnaJ-like chaperone polypeptide.

According to the present invention, there is provided a method for increasing content of any one or more fine chemical listed in table FC relative to control plants in plants grown under non-stress or stress conditions, wherein stress conditions are preferably under conditions of limited water availability, particularly drought conditions, which method comprises modulating expression in a plant of a nucleic acid encoding a DnaJ-like chaperone polypeptide.

Further provided by the present invention are methods for increasing yield-related traits of plants under abiotic environmental stress conditions and/or non-stress conditions, and for increasing content of any one or more fine chemical listed in table FC relative to control plants in plants grown under non-stress or stress conditions which method comprises modulating expression in a plant of a nucleic acid encoding a DnaJ-like chaperone polypeptide.

Performance of the methods of the invention gives plants grown under conditions of drought, increased yield and/or fine chemical content of any one or more fine chemical listed in table FC, relative to control plants grown under comparable conditions. Therefore, according to the present invention, there is provided a method for increasing yield and/or fine chemical content of any one or more fine chemical listed in table FC, in plants grown under conditions of drought which method comprises modulating expression in a plant of a nucleic acid encoding a DnaJ-like chaperone polypeptide.

Performance of the methods of the invention gives plants grown under conditions of nutrient deficiency, particularly under conditions of nitrogen deficiency, increased yield and/or fine chemical content of any one or more fine chemical listed in table FC, relative to control plants grown under comparable conditions. Therefore, according to the present invention, there is provided a method for increasing yield and/or fine chemical content of any one or more fine chemical listed in table FC, in plants grown under conditions of nutrient deficiency, which method comprises modulating expression in a plant of a nucleic acid encoding a DnaJ-like chaperone polypeptide.

Performance of the methods of the invention gives plants grown under conditions of salt stress, increased yield and/or fine chemical content of any one or more fine chemical listed in table FC, relative to control plants grown under comparable conditions. Therefore, according to the present invention, there is provided a method for increasing yield and/or fine chemical content of any one or more fine chemical listed in table FC, in plants grown under conditions of salt stress, which method comprises modulating expression in a plant of a nucleic acid encoding a DnaJ-like chaperone polypeptide.

The invention also provides genetic constructs and vectors to facilitate introduction and/or expression in plants of nucleic acids encoding DnaJ-like chaperone polypeptides. The gene constructs may be inserted into vectors, which may be commercially available, suitable for transforming into plants and suitable for expression of the gene of interest in the transformed cells. The invention also provides use of a gene construct as defined herein in the methods of the invention.

More specifically, the present invention provides a construct comprising:
(a) a nucleic acid encoding a DnaJ-like chaperone polypeptide as defined above;
(b) one or more control sequences capable of driving expression of the nucleic acid sequence of (a); and optionally
(c) a transcription termination sequence.

Preferably, the nucleic acid encoding a DnaJ-like chaperone polypeptide is as defined above. The term “control sequence” and “termination sequence” are as defined herein.

The invention furthermore provides plants transformed with a construct as described above. In particular, the invention provides plants transformed with a construct as described above, which plants have increased yield-related traits as described herein.

Plants are transformed with a vector comprising any of the nucleic acids described above. The skilled artisan is well aware of the genetic elements that must be present on the vector in order to successfully transform, select and propagate host cells containing the sequence of interest. The sequence of interest is operably linked to one or more control sequences (at least to a promoter) in the vectors of the invention.

In one embodiment the plants of the invention are transformed with an expression cassette comprising any of the nucleic acids described above. The skilled artisan is well aware of the genetic elements that must be present on the expression cassette in order to successfully transform, select and propagate host cells containing the sequence of interest. In the expression cassettes of the invention the sequence of interest is operably linked to one or more control sequences (at least to a promoter). The promoter in such an expression cassette may be a non-native promoter to the nucleic acid described above, i.e. a promoter not regulating the expression of said nucleic acid in its native surrounding. In a further embodiment the expression cassettes of the invention confer increased yield or yield related trait(s) to a living plant cell when they have been introduced into said plant cell and result in expression of the nucleic acid as defined above, comprised in the expression cassette(s).

The expression cassettes of the invention may be comprised in a host cell, plant cell, seed, agricultural product or plant.

Advantageously, any type of promoter, whether natural or synthetic, may be used to drive expression of the nucleic acid sequence. In one embodiment the promoter is of plant origin. A constitutive promoter is particularly useful in the methods. Preferably the constitutive promoter is a ubiquitous constitutive promoter of medium strength or high strength. See the “Definitions” section herein for definitions of the various promoter types.

It should be clear that the applicability of the present invention is not restricted to the DnaJ-like chaperone polypeptide-encoding nucleic acid represented by SEQ ID NO: 1 or 41, preferably by SEQ ID NO: 1, nor is the applicability of the invention restricted to expression of a DnaJ-like chaperone polypeptide-encoding nucleic acid when driven by a constitutive promoter.

The constitutive promoter is preferably a medium or high strength promoter. In one embodiment it is a plant derived promoter, e.g. a promoter of plant chromosomal origin, such as a GOS2 promoter, PcUbi promoter, USP promoter or a promoter of substantially the same strength and having substantially the same expression pattern (a functionally equivalent promoter).

In another embodiment the constitutive promoter is a promoter derived from the CaMV35S promoter, e.g. the Big35S or the Super promoter. See the explanations to table III below for more information on the USP, PcUbi, Super and Big35S promoters.

See the “Definitions” section herein for further examples of constitutive promoters.

Optionally, one or more terminator sequences may be used in the construct introduced into a plant. Preferably, the construct comprises an expression cassette comprising a constitutive promoter, e.g. the Big35S promoter, operably linked to the nucleic acid encoding the DnaJ-like chaperone polypeptide. More preferably, the construct comprises a terminator, e.g. the t-Nos or zein terminator (t-zein) linked to the 3′ end of the DnaJ-like chaperone coding sequence. Furthermore, one or more sequences encoding selectable markers may be present on the construct introduced into a plant.

According to a preferred feature of the invention, the modulated expression is increased expression. Methods for increasing expression of nucleic acids or genes, or gene products, are well documented in the art and examples are provided in the definitions section.

As mentioned above, a preferred method for modulating expression of a nucleic acid encoding a DnaJ-like chaperone polypeptide is by introducing and expressing in a plant a nucleic acid encoding a DnaJ-like chaperone polypeptide; however the effects of performing the method, i.e. enhancing yield-related traits may also be achieved using other well known techniques, including but not limited to T-DNA activation tagging, TILLING, homologous recombination. A description of these techniques is provided in the definitions section.

The invention also provides a method for the production of transgenic plants having enhanced yield-related traits under abiotic environmental stress conditions and/or non-stress conditions, preferably under conditions of limited water availability, more preferably under conditions of drought, and/or increased content of any one or more fine chemical listed in table FC relative to control plants, comprising introduction and expression in a plant of any nucleic acid encoding a DnaJ-like chaperone polypeptide as defined hereinabove.

More specifically, the present invention provides a method for the production of transgenic plants having enhanced yield-related traits, particularly increased biomass and/or seed yield, under abiotic environmental stress conditions and/or non-stress conditions, preferably under conditions of limited water availability, more preferably under conditions of drought, and/or increased content of any one or more fine chemical listed in table FC relative to control plants, which method comprises:
(i) introducing and expressing in a plant or plant cell a DnaJ-like chaperone polypeptide-encoding nucleic acid or a genetic construct comprising a DnaJ-like chaperone polypeptide-encoding nucleic acid; and
(ii) cultivating the plant cell under conditions promoting plant growth and development.

Cultivating the plant cell under conditions promoting plant growth and development, may or may not include regeneration and or growth to maturity.

The nucleic acid of (i) may be any of the nucleic acids capable of encoding a DnaJ-like chaperone polypeptide as defined herein.

The nucleic acid may be introduced directly into a plant cell or into the plant itself (including introduction into a tissue, organ or any other part of a plant). According to a preferred feature of the present invention, the nucleic acid is preferably introduced into a plant by transformation. The term “transformation” is described in more detail in the “definitions” section herein.

In one embodiment the present invention clearly extends to any harvestable part of a plant with increased content of any one or more fine chemical listed in table FC relative to harvestable parts from control plants, produced by any of the methods described herein, and to all products with increased content of any one or more fine chemical listed in table FC thereof. The harvestable parts thereof comprise a nucleic acid transgene encoding a DnaJ-like chaperone polypeptide as defined above.

The present invention also extends in another embodiment to harvestable parts with increased content of any one or more fine chemical listed in table FC comprising the nucleic acid molecule of the invention in a plant expression cassette or a plant expression construct.

In yet another embodiment the harvestable parts of the invention are non-propagative cells, e.g. the cells can not be used to regenerate a whole plant from this cell as a whole using standard cell culture techniques, this meaning cell culture methods but excluding in-vitro nuclear, organelle or chromosome transfer methods. While plants cells generally have the characteristic of totipotency, some plant cells can not be used to regenerate or propagate intact plants from said cells. In one embodiment of the invention the plant cells of the invention are such cells.

In another embodiment the harvestable parts of the invention are harvestable parts that do not sustain themselves through photosynthesis by synthesizing carbohydrate and protein from such inorganic substances as water, carbon dioxide and mineral salt, i.e. they may be deemed non-plant variety. In a further embodiment the harvestable parts of the invention are non-plant variety and non-propagative.

In one embodiment, an increase of myo-inositol in a non-human organism, as compared to a corresponding non-transformed wild type non-human organism, is conferred in the process of the invention, if the activity of a polypeptide showing the activity of a molecular chaperone, or if the activity of the polypeptide Ynl064c, preferably represented by SEQ ID NO: 2 or 42, preferably SEQ ID NO: 2, or a homolog or fragment thereof, or if the activity of a polypeptide encoded by a nucleic acid molecule comprising the nucleic acid SEQ ID NO: 1 or 41, preferably SEQ ID NO: 1, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, column 5 or 7 in the respective same line as the nucleic acid molecule SEQ ID NO: 1 or 41, preferably SEQ ID NO: 1 or polypeptide SEQ ID NO: 2 or 42, preferably SEQ ID NO: 2, respectively, or a homolog or a fragment thereof, is increased or generated, or if the activity molecular chaperone is increased or generated in a non-human organism, like a microorganism or a plant cell, plant or part thereof, especially with non-targeted localization, whereby the respective line disclose in table R1 the fine chemical myo-inositol. For example, an increase of the myo-inositol of at least 1 percent, particularly in a range of 28 to 50-percent is conferred as compared to a corresponding non-transformed wild type non-human organism.

Accordingly, in another embodiment, an increase of sucrose in a non-human organism, as compared to a corresponding non-transformed wild type non-human organism, is conferred in the process of the invention, if the activity of a polypeptide showing the activity of a molecular chaperone, or if the activity of the polypeptide Ynl064c, preferably represented by SEQ ID NO: 2 or 42, preferably SEQ ID NO: 2, or a homolog or fragment thereof, or if the activity of a polypeptide encoded by a nucleic acid molecule comprising the nucleic acid SEQ ID NO: 1 or 41, preferably SEQ ID NO: 1, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV column 5 or 7 in the respective same line as the nucleic acid molecule SEQ ID NO: 1 or 41, preferably SEQ ID NO: 1 or polypeptide SEQ ID NO: 2 or 42, preferably SEQ ID NO: 2, respectively, or a homolog or a fragment thereof, is increased or generated, or if the activity molecular chaperone is increased or generated in a non-human organism, like a microorganism or a plant cell, plant or part thereof, especially with non-targeted localization, whereby the respective line disclose in table R1 the fine chemical sucrose. For example, an increase of the sucrose of at least 1 percent, particularly in a range of 25 to 31-percent is conferred as compared to a corresponding non-transformed wild type non-human organism.

In a further embodiment, an increase of linoleic acid in a non-human organism, as compared to a corresponding non-transformed wild type non-human organism, is conferred in the process of the invention, if the activity of a polypeptide showing the activity of a molecular chaperone, or if the activity of the polypeptide Ynl064c, preferably represented by SEQ ID NO: 2 or 42, preferably SEQ ID NO: 2, or a homolog or fragment thereof, or if the activity of a polypeptide encoded by a nucleic acid molecule comprising the nucleic acid SEQ ID NO: 1 or 41, preferably SEQ ID NO: 1, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, column 5 or 7 in the respective same line as the nucleic acid molecule SEQ ID NO: 1 or 41, preferably SEQ ID NO: 1 or polypeptide SEQ ID NO: 2 or 42, preferably SEQ ID NO: 2, respectively, or a homolog or a fragment thereof, is increased or generated, or if the activity molecular chaperone is increased or generated in a non-human organism, like a microorganism or a plant cell, plant or part thereof, especially with non-targeted localization, whereby the respective line disclose in table R1 the fine chemical linoleic acid. For example, an increase of the linoleic acid of at least 1 percent, particularly in a range of 15 to 25-percent is conferred as compared to a corresponding non-transformed wild type non-human organism.

In a further embodiment, an increase of linolenic acid in a non-human organism, as compared to a corresponding non-transformed wild type non-human organism, is conferred in the process of the invention, if the activity of a polypeptide showing the activity of a molecular chaperone, or if the activity of the polypeptide Ynl064c, preferably represented by SEQ ID NO: 2 or 42, preferably SEQ ID NO: 2, or a homolog or fragment thereof, or if the activity of a polypeptide encoded by a nucleic acid molecule comprising the nucleic acid SEQ ID NO: 1 or 41, preferably SEQ ID NO: 1, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, column 5 or 7 in the respective same line as the nucleic acid molecule SEQ ID NO: 1 or 41, preferably SEQ ID NO: 1 or polypeptide SEQ ID NO: 2 or 42, preferably SEQ ID NO: 2, respectively, or a homolog or a fragment thereof, is increased or generated, or if the activity molecular chaperone is increased or generated in a non-human organism, like a microorganism or a plant cell, plant or part thereof, especially with non-targeted localization, whereby the respective line disclose in table R1 the fine chemical linolenic acid. For example, an increase of the linolenic acid of at least 1 percent, particularly in a range of 13 to 24-percent is conferred as compared to a corresponding non-transformed wild type non-human organism.

A further embodiment of this invention is related to genes which increase or generate the production of the fine chemical linoleic acid in plant cells, plants or part thereof. Phenotypes thereto are associated with yield of plants (=yield related phenotypes). In accordance with the invention, therefore, the respective genes identified in Table I, columns 5 or 7, wherein for the corresponding lead gene in table R1, column 5 linoleic acid is mentioned, especially the coding region thereof, or homologs or fragments thereof, may be employed to enhance any yield-related phenotype.

The fine chemical myo-inositol may protect plant cells from limitations in water availability and hence may increase yield-related phenotypes under non-stress and/or under stress conditions.

In accordance with the invention, therefore, the respective genes identified in Table I, columns 5 or 7, wherein for the corresponding lead gene in table R1, column 5 myo-inositol is mentioned, especially the coding region thereof, or homologs or fragments thereof, may be employed to enhance any yield-related phenotype.

Further, in crops with harvestable parts harvested mainly for their sugar content, such as sugarcane or sugar beet, an increase in sugar content, and particular content of the fine chemical sucrose will directly improve the yield of the relevant harvestable parts.

In accordance with the invention, therefore, the respective genes identified in Table I, columns 5 or 7, wherein for the corresponding lead gene in table R1, column 5 sucrose is mentioned, especially the coding region thereof, or homologs or fragments thereof, may be employed to enhance any yield-related phenotype.

Increased yield may be determined in field trials of transgenic plants and suitable control plants. Alternatively, a transgene's ability to increase yield may be determined in a model plant. An increased yield phenotype may be determined in the field test or in a model plant by measuring any one or any combination of the following phenotypes, in comparison to a control plant: yield of dry harvestable parts of the plant, yield of dry aerial harvestable parts of the plant, yield of underground dry harvestable parts of the plant, yield of fresh weight harvestable parts of the plant, yield of aerial fresh weight harvestable parts of the plant yield of underground fresh weight harvestable parts of the plant, yield of the plant's fruit (both fresh and dried), grain dry weight, yield of seeds (both fresh and dry), and the like.

The most basic yield-related phenotype is increased yield associated with the presence of the gene or a homolog or a fragment thereof as a transgene in the plant, i.e., the intrinsic yield of the plant. Intrinsic yield capacity of a plant can be, for example, manifested in a field test or in a model system by demonstrating an improvement of seed yield (e.g. in terms of increased seed/grain size, increased ear number, increased seed number per ear, improvement of seed filling, improvement of seed composition, embryo and/or endosperm improvements, and the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under non-stressed conditions), improvement in plant architecture. In accordance with the invention, the respective genes identified in Table 1, columns 5 or 7, especially the coding region thereof, or homologs or fragments thereof, wherein in the respective line of table R1 linoleic acid, myo-inositol and/or sucrose is mentioned, may be employed to enhance intrinsic yield capacity.

Increased yield-related phenotypes may also be measured to determine tolerance to abiotic i.e. environmental stress. In one embodiment “abiotic stress”, “environmental stress” and “abiotic environmental stress” are used interchangeably, also when referring to tolerance to such stress Abiotic stresses include drought, low temperature, nutrient deficiency, salinity, osmotic stress, shade, high plant density, mechanical stresses, and oxidative stress, preferably drought and reduced water availability, and yield-related phenotypes are encompassed by tolerance to such abiotic stresses. Additional phenotypes that can be monitored to determine enhanced tolerance to abiotic environmental stress include, without limitation, wilting; leaf browning; loss of turgor, which results in drooping of leaves or needles stems, and flowers; drooping and/or shedding of leaves or needles; the leaves are green but leaf angled slightly toward the ground compared with controls; leaf blades begun to fold (curl) inward; premature senescence of leaves or needles; loss of chlorophyll in leaves or needles and/or yellowing. Any of the yield-related phenotypes described above may be monitored in field tests or in model plants to demonstrate that a transgenic plant has increased tolerance to abiotic environmental stress.

A polypeptide conferring a yield-increasing activity can be encoded by a respective nucleic acid sequence as shown in Table I, column 5 or 7, and/or comprises or consists of a respective polypeptide as depicted in Table II, column 5 and 7, and/or can be amplified with the respective primer set shown in Table III, column 7, in case in the corresponding line in Table R1 linoleic acid, myo-inositol and/or sucrose is indicated.

“Improved adaptation” to environmental stress like e.g. freezing and/or chilling temperatures refers to an improved plant performance under environmental stress conditions.

A modification, for example an increase, can be caused by endogenous or exogenous factors. For example, an increase in activity in an organism or a part thereof can be caused by adding a gene product or a precursor or an activator or an agonist to the media or nutrition or can be caused by introducing said subjects into an organism, transient or stable. Furthermore such an increase can be reached by the introduction of the respective inventive nucleic acid sequence or the encoded protein in the correct cell compartment for example into the nucleus or cytoplasmic respectively or into plastids either by transformation and/or targeting.

In one embodiment the term “yield” as used herein generally refers to a measurable produce from a plant, particularly a crop. Yield and yield increase (in comparison to a non-transformed starting or wild-type plant) can be measured in a number of ways, and it is understood that a skilled person will be able to apply the correct meaning in view of the particular embodiments, the particular crop concerned and the specific purpose or application concerned. The terms “improved yield” or “increased yield” can be used interchangeable.

For example, enhanced or increased “yield” refers to one or more yield parameters selected from the group consisting of biomass yield, dry biomass yield, aerial dry biomass yield, underground dry biomass yield, fresh-weight biomass yield, aerial fresh-weight biomass yield, underground fresh-weight biomass yield; enhanced yield of harvestable parts, either dry or fresh-weight or both, either aerial or underground or both; enhanced yield of crop fruit, either dry or fresh-weight or both, either aerial or underground or both; and enhanced yield of seeds, either dry or fresh-weight or both, either aerial or underground or both. Preferably the above ground biomass yield, and/or the beet biomass, tuber biomass and/or root biomass yield is increased.

Accordingly, the yield of a plant can be increased by improving one or more of the yield-related phenotypes.

Such yield-related phenotypes or traits of a plant the improvement of which results in increased yield comprise, without limitation, the increase of the intrinsic yield capacity of a plant, and/or increased stress tolerance, e.g. improved drought tolerance or improved nutrient use efficiency. For example, yield refers to biomass yield, e.g. to dry weight biomass yield and/or fresh-weight biomass yield. Biomass yield refers to the aerial or underground parts of a plant or to parts in contact with the ground or partly inserted in the ground like beets, depending on the specific circumstances (test conditions, specific crop of interest, application of interest, and the like). In one embodiment, biomass yield refers to the aerial and underground parts. Biomass yield may be calculated as fresh-weight, dry weight or a moisture adjusted basis. Biomass yield may be calculated on a per plant basis or in relation to a specific area (e.g. biomass yield per acre/square meter/or the like).

For example, the term “increased yield” means that a plant, exhibits an increased growth rate, under conditions of abiotic environmental stress, compared to the corresponding wild-type plant.

An increased growth rate may be reflected inter alia by or confers an increased biomass production of the whole plant, or an increased biomass production of the aerial parts of a plant, or an increased biomass production of parts in contact with the ground or partly inserted in the ground like beets, or by an increased biomass production of the underground parts of a plant, or by an increased biomass production of parts of a plant, like stems, leaves, blossoms, fruits, and/or seeds. Increased yield includes higher fruit yields, higher seed yields, higher fresh matter production, and/or higher dry matter production.

In one embodiment the term “increased yield” means that the plant, exhibits a prolonged growth under conditions of abiotic environmental stress, as compared to the corresponding, e.g. non-transformed, wild type organism. A prolonged growth comprises survival and/or continued growth of the plant, at the moment when the non-transformed wild type organism shows visual symptoms of deficiency and/or death.

Said increased yield can typically be achieved by enhancing or improving, one or more yield related traits of the plant. Such yield-related traits of a plant comprise, without limitation, the increase of the intrinsic yield capacity of a plant, and/or increased stress tolerance, in particular increased abiotic stress tolerance, like for example improved nutrient use efficiency, e.g. nitrogen use efficiency, water use efficiency.

Intrinsic yield capacity of a plant can be, for example, manifested by improving the specific (intrinsic) biomass yield (e.g. in terms of increased shoot, root or beet size, improvement of beet, root or shoot composition, or the like); modification and improvement of inherent growth and development mechanisms of a plant (such as plant height, plant growth rate, leaf number, leaf position on the plant, number of internodes, efficiency of nodulation and nitrogen fixation, efficiency of carbon assimilation, improvement of seedling vigour/early vigour, enhanced efficiency of germination (under stressed or non-stressed conditions), improvement in plant architecture, cell cycle modifications, photosynthesis modifications, various signaling pathway modifications, modification of transcriptional regulation, modification of translational regulation, modification of enzyme activities, and the like); and/or the like.

The improvement or increase of stress tolerance of a plant can for example be manifested by improving or increasing a plant's tolerance against stress, particularly abiotic stress. In the present application, abiotic stress refers generally to abiotic environmental conditions a plant is typically confronted with, including, but not limited to, drought (tolerance to drought may be achieved as a result of improved water use efficiency), heat, low temperatures and cold conditions (such as freezing and chilling conditions), nutrient depletion, salinity, osmotic stress, shade, high plant density, mechanical stress, oxidative stress, and the like.

Accordingly, this invention provides respective measures and methods to produce plants with increased yield, e.g. genes conferring an increased yield-related trait, for example enhanced tolerance to abiotic environmental stress, for example an increased drought tolerance and/or low temperature tolerance and/or an increased nutrient use efficiency, intrinsic yield and/or another increased yield-related trait, upon expression or over-expression, especially under drought conditions. Accordingly, the present invention provides such genes in case in Table R1 linoleic acid, myo-inositol and/or sucrose is indicated. In particular, such genes are described in column 5 as well as in column 7 of Tables I, especially the coding region thereof, or homologs or fragments thereof, in case linoleic acid, myo-inositol and/or sucrose is indicated in table R1 or the respective polypeptides are described in column 5 as well as in column 7 of Table II, or homologs or fragments thereof, in case linoleic acid, myo-inositol and/or sucrose is indicated in table R1.

Accordingly, the present invention provides respective transgenic plants showing one or more improved yield-related traits as compared to the corresponding control or the wild type plant and methods for producing such transgenic plants with increased yield in case in table R1 linoleic acid, myo-inositol and/or sucrose is indicated.

In one embodiment, one or more of said yield-increasing activities are increased by increasing the amount and/or the specific activity of one or more proteins listed in Table I, column 5 or 7 in a compartment of a cell indicated in Table I, column 6, in case in table R1 linoleic acid, myo-inositol and/or sucrose is indicated.

Accordingly to present invention, the yield of the plant of the invention is increased by improving one or more of the yield-related traits as defined herein. Said increased yield in accordance with the present invention can typically be achieved by enhancing or improving, in comparison to a control or wild-type plant, one or more yield-related traits of said plant.

Such yield-related traits of a plant the improvement of which results in increased yield comprise, without limitation, the increase of the intrinsic yield capacity of a plant, and/or increased stress tolerance, e.g. improved nutrient use efficiency, like nitrogen use efficiency; especially enhanced yield capacity under drought stress or water limitation.

The activity of the gene product of the nucleic acid sequence of Ynl064c from Saccharomyces cerevisiae, e.g. as shown in the respective line in column 5 of Table I, is the activity of molecular chaperone.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “molecular chaperone”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), preferably the coding region thereof, or a homolog or a fragment thereof, and being depicted in the same respective line as said Ynl064c, or a functional equivalent or a homolog thereof as shown in column 7 of Table I, preferably the coding region thereof, and preferably the activity is increased non-targeted, or
(b) a polypeptide comprising a polypeptide, a consensus sequence or at least a polypeptide motif as shown in the respective line in column 5 of Table II or in column 7 of Table IV, respectively, and being depicted in the same respective line as said Ynl064c, or a functional equivalent or a homolog thereof as depicted in column 7 of Table II, and being depicted in the same respective line as said Ynl064c, and preferably the activity is increased non-targeted, whereby the respective line disclose in table R1 the fine chemical myo-inositol.

Accordingly, in one embodiment, the molecule which activity is to be increased in the process of the invention is the gene product with an activity as a “molecular chaperone”, preferably it is the molecule of section (a) or (b) of this paragraph.

In particular, it was observed that in plants, especially in Arabidopsis thaliana, increasing or generating the activity of a gene product non-targeted with the activity of a “molecular chaperone”, preferably being encoded by a gene comprising the nucleic acid sequence SEQ ID NO: 1 or 41, preferably SEQ ID NO: 1, preferably the coding region thereof, conferred the production of or the increase in myo-inositol compared with the wild type control.

Accordingly, in a further embodiment, the process of the present invention for producing sucrose in a non-human organism, like a microorganism or a plant or a part thereof, comprises increasing or generating the activity of a gene product with the activity of a gene product conferring the activity of “molecular chaperone”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), preferably the coding region thereof, or a homolog or a fragment thereof, and being depicted in the same respective line as said Ynl064c, or a functional equivalent or a homolog thereof as shown in column 7 of Table I, preferably the coding region thereof, and preferably the activity is increased non-targeted, or
(b) a polypeptide comprising a polypeptide, a consensus sequence or at least a polypeptide motif as shown in the respective line in column 5 of Table II or in column 7 of Table IV, respectively, and being depicted in the same respective line as said Ynl064c, or a functional equivalent or a homolog thereof as depicted in column 7 of Table II, and being depicted in the same respective line as said Ynl064c, and preferably the activity is increased non-targeted, whereby the respective line disclose in table R1 the fine chemical sucrose.

Accordingly, in one embodiment, the molecule which activity is to be increased in the process of the invention is the gene product with an activity as a “molecular chaperone”, preferably it is the molecule of section (a) or (b) of this paragraph.

In particular, it was observed that in plants, especially in Arabidopsis thaliana, increasing or generating the activity of a gene product non-targeted with the activity of a “molecular chaperone”, preferably being encoded by a gene comprising the nucleic acid sequence SEQ ID NO: 1 or 41, preferably SEQ ID NO: 1, preferably the coding region thereof, conferred the production of or the increase in sucrose compared with the wild type control.

Accordingly, in a further embodiment, the process of the present invention for producing linoleic acid in a non-human organism, like a microorganism or a plant or a part thereof, comprises increasing or generating the activity of a gene product with the activity of a gene product conferring the activity of “molecular chaperone”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical linoleic acid), preferably the coding region thereof, or a homolog or a fragment thereof, and being depicted in the same respective line as said Ynl064c, or a functional equivalent or a homolog thereof as shown in column 7 of Table I, preferably the coding region thereof, and preferably the activity is increased non-targeted, or
(b) a polypeptide comprising a polypeptide, a consensus sequence or at least a polypeptide motif as shown in the respective line in column 5 of Table II or in column 7 of Table IV, respectively, and being depicted in the same respective line as said Ynl064c, or a functional equivalent or a homolog thereof as depicted in column 7 of Table II, and being depicted in the same respective line as said Ynl064c, and preferably the activity is increased non-targeted, whereby the respective line disclose in table R1 the fine chemical linoleic acid.

Accordingly, in one embodiment, the molecule which activity is to be increased in the process of the invention is the gene product with an activity as a “molecular chaperone”, preferably it is the molecule of section (a) or (b) of this paragraph.

In particular, it was observed that in plants, especially in Arabidopsis thaliana, increasing or generating the activity of a gene product non-targeted with the activity of a “molecular chaperone”, preferably being encoded by a gene comprising the nucleic acid sequence SEQ ID NO: 1 or 41, preferably SEQ ID NO: 1, preferably the coding region thereof, conferred the production of or the increase in linoleic acid compared with the wild type control.

Accordingly, in a further embodiment, the process of the present invention for producing linolenic acid in a non-human organism, like a microorganism or a plant or a part thereof, comprises increasing or generating the activity of a gene product with the activity of a gene product conferring the activity of “molecular chaperone”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical linolenic acid), preferably the coding region thereof, or a homolog or a fragment thereof, and being depicted in the same respective line as said Ynl064c, or a functional equivalent or a homolog thereof as shown in column 7 of Table I, preferably the coding region thereof, and preferably the activity is increased non-targeted, or
(b) a polypeptide comprising a polypeptide, a consensus sequence or at least a polypeptide motif as shown in the respective line in column 5 of Table II or in column 7 of Table IV, respectively, and being depicted in the same respective line as said

Ynl064c, or a functional equivalent or a homolog thereof as depicted in column 7 of Table II, and being depicted in the same respective line as said Ynl064c, and preferably the activity is increased non-targeted, whereby the respective line disclose in table R1 the fine chemical linolenic acid.

Accordingly, in one embodiment, the molecule which activity is to be increased in the process of the invention is the gene product with an activity as a “molecular chaperone”, preferably it is the molecule of section (a) or (b) of this paragraph.

In particular, it was observed that in plants, especially in Arabidopsis thaliana, increasing or generating the activity of a gene product non-targeted with the activity of a “molecular chaperone”, preferably being encoded by a gene comprising the nucleic acid sequence SEQ ID NO: 1 or 41, preferably SEQ ID NO: 1, preferably the coding region thereof, conferred the production of or the increase in linolenic acid compared with the wild type control.

TABLE FC
Fine chemicals increased in plants and/or plant cells
and/or harvestable parts by the inventive processes

| Fine chemical | Belonging to the group of |
|---|---|
| Sucrose | Carbohydrates, saccharides |
| Myo-inositol | Carbohydrates, saccharides |
| Linoleic acid | Fatty acids |
| Linolenic acid | Fatty acids |

Thus, in one embodiment, the present invention provides a process of the production of any one or more fine chemical listed in table FC, by increasing or generating one or more activities of DnaJ-like chaperone which is conferred by one or more POIs or the gene product of one or more POI-genes, for example by the gene product of a nucleic acid sequences comprising a polynucleotide selected from the group as shown in Table I column 5 or 7, (preferably by the coding region thereof), or a homolog or a fragment thereof, e.g. or by one or more proteins each comprising a polypeptide encoded by one or more nucleic acid sequences selected from the group as shown in Table I column 5 or 7, (preferably by the coding region thereof), or a homolog or a fragment thereof, or by one or more protein(s) each comprising a polypeptide selected from the group as depicted in Table II column 5 and 8, or a homolog thereof, or a protein comprising a sequence corresponding to the consensus sequence or comprising at least one polypeptide motif as shown in Table IV column 7.

As mentioned, the process for the production of the fine chemical according to the present invention, in particular showing a generation or an increase of the respective fine chemical in a non-human organism or a part thereof as compared to a corresponding wild-type non-human organism or part thereof, can be mediated by one or more DnaJ-like chaperone-genes or DnaJ-like chaperones.

In an embodiment, the process comprises increasing or generating the activity of one or more polypeptides having said activity, e.g. by generating or increasing the amount and/or specific activity in the cell or a compartment of a cell of one of more POI, especially DnaJ-like chaperone for example of the respective polypeptide as depicted in Table II column 5 and 8, or a homolog or a fragment thereof, or the respective polypeptide comprising a sequence corresponding to the consensus sequences as shown in Table IV column 7, or the respective polypeptide comprising at least one polypeptide motif as depicted in Table IV column 7.

A further embodiment of the present invention relates to a process for the production of any one or more fine chemicals listed in table FC, which comprises
(a) increasing or generating the activity of a DnaJ-like chaperone non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of any one or more fine chemicals listed in table FC or a composition comprising any one or more fine chemicals listed in table FC in said non-human organism or in the culture medium surrounding said non-human organism.

A further embodiment of the present invention relates to a process for the production of any one or more fine chemicals listed in table FC, which comprises
(a) increasing or generating the activity of a polypeptide comprising a polypeptide as depicted in the respective line in column 5 or 7 of Table II or a homolog or a fragment thereof, a consensus sequence or at least one polypeptide motif as depicted in the respective line in column 7 of Table IV or
   increasing or generating the activity of an expression product of one or more nucleic acid molecule(s) comprising a polynucleotide as depicted in the respective line in column 5 or 7 of Table I preferably the coding region thereof, or a homolog or a fragment thereof;
   non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of any one or more fine chemicals listed in table FC, or a composition comprising any one or more fine chemicals listed in table FC in said non-human organism or in the culture medium surrounding said non-human organism.

A further embodiment of the present invention relates to a process for the production of any one or more fine chemicals listed in table FC, which comprises
(a) increasing or generating one or more activities selected from the group consisting of DnaJ-like chaperone in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of any one or more fine chemicals listed in table FC or a composition comprising any one or more fine chemicals listed in table FC in said non-human organ-ism or in the culture medium surrounding said non-human organism.

A further embodiment of the present invention relates to a process for the production of any one or more fine chemicals listed in table FC, which comprises
(a1) increasing or generating the activity of a polypeptide comprising a polypeptide as depicted in the respective line in column 5 or 7 of Table II or a homolog or fragment thereof, a consensus sequence or at least one polypeptide motif as depicted in column 7 of Table IV or
   increasing or generating the activity of an expression product of one or more nucleic acid molecule(s) comprising a polynucleotide as depicted in the respective line in column 5 or 7 of Table I preferably the coding region thereof, or a homolog or a fragment thereof;
   in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 7 of Table II or a homolog or a fragment thereof, a consensus sequence or at least one polypeptide motif as depicted in the respective line in column 7 of Table IV which is joined to a transit peptide; or
   increasing or generating the activity of an expression product of one or more nucleic acid molecule(s) comprising a polynucleotide as depicted in the respective line in column 5 or 7 of Table I preferably the coding region thereof, or a homolog or a fragment thereof, which is joined to a nucleic acid sequence encoding an organelle localization sequence, preferably a plastid or a mitochondrion localization sequence, especially a plastid localization sequence;
   in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 7 of Table II or a homolog or a fragment thereof, a consensus sequence or at least one polypeptide motif as depicted in the respective line in column 7 of Table IV or
   increasing or generating the activity of an expression product of one or more nucleic acid molecule(s) comprising a polynucleotide as depicted in the respective line in column 5 or 7 of Table I preferably the coding region thereof, or a homolog or a fragment thereof;
   in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof; and
(b) growing the non-human organism under conditions which permit the production of any one or more fine chemicals listed in table FC, or a composition comprising any one or more fine chemicals listed in table FC in said non-human organism or in the culture medium surrounding said non-human organism.

Preferably, the present invention relates to a process for the production of any one or more fine chemicals listed in table FC, which comprises
(a) increasing or generating the activity of a DnaJ-like chaperone in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of any one or more fine chemicals listed in table FC or a composition comprising any one or more fine chemicals listed in table FC in said non-human organism or in the culture medium surrounding said non-human organism.

Accordingly, the present invention relates to a process for the production of any one or more fine chemicals listed in table FC, which comprises
(a) increasing or generating the activity of a polypeptide comprising a polypeptide as depicted in the respective line in column 5 or 7 of Table II or a homolog or a fragment thereof, a consensus sequence or at least one polypeptide motif as depicted in the respective line in column 7 of Table IV or
   increasing or generating the activity of an expression product of one or more nucleic acid molecule(s) comprising a polynucleotide as depicted in the respective line in column 5 or 7 of Table I preferably the coding region thereof, or a homolog or a fragment thereof;
   in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of any one or more fine chemicals listed in table FC, or a composition comprising any one or more fine chemicals listed in table FC in said non-human organism or in the culture medium surrounding said non-human organism.

Throughout this application a reference to any one or more fine chemical as listed in table FC is intended to mean sucrose, myo-inositol, linoleic acid or linolenic acid, or any combination thereof.

In one embodiment the fine chemical generated or increased by the inventive processes in a plant, plant cell, harvestable part or agricultural product is sucrose, or a combination selected from the group consisting of:
1. sucrose and myo-inositol,
2. sucrose and linoleic acid,
3. sucrose and linolenic acid, and
4. sucrose and myo-inositol and linoleic acid and linolenic acid.

In another embodiment the fine chemical generated or increased by the inventive processes in a plant, plant cell, harvestable part or agricultural product is myo-inositol, or a combination selected from the group consisting of:
1. myo-inositol and sucrose,
2. myo-inositol and linoleic acid,
3. myo-inositol and linolenic acid, and
4. sucrose and myo-inositol and linoleic acid and linolenic acid.

In another embodiment the fine chemical generated or increased by the inventive processes in a plant, plant cell, harvestable part or agricultural product is linoleic acid, or a combination selected from the group consisting of:
1. linoleic acid and sucrose,
2. myo-inositol and linoleic acid,
3. linoleic acid and linolenic acid, and
4. sucrose and myo-inositol and linoleic acid and linolenic acid.

In another embodiment the fine chemical generated or increased by the inventive processes in a plant, plant cell, harvestable part or agricultural product is linolenic acid, or a combination selected from the group consisting of:
1. linolenic acid and sucrose,
2. myo-inositol and linolenic acid,
3. linoleic acid and linolenic acid, and
4. sucrose and myo-inositol and linoleic acid and linolenic acid.

Owing to the introduction of a gene or a plurality of genes conferring the expression of the DnaJ-like chaperone encoding molecule or the DnaJ-like chaperone polypeptide, for example the nucleic acid construct mentioned below, or encoding the protein as shown in the respective line in Table II column 5 or 7, or homologs or fragments thereof, into a non-human organism alone or in combination with other genes, it is possible not only to increase the biosynthetic flux towards the end product, but also to increase, modify or create de novo an advantageous, preferably novel metabolites composition in the non-human organism, e.g. an advantageous composition comprising a higher content of (from a viewpoint of nutritional physiology limited) any one or more fine chemical listed in table FC and if desired other fatty acid and/or saccharides, and/or other metabolites, in free or bound form.

In a further embodiment the activity of the polypeptide comprising a polypeptide as depicted in the respective line in column 5 or 7 of Table II or a homolog or a fragment thereof, a consensus sequence or at least one polypeptide motif as depicted in the respective line in column 7 of Table IV is increased or generated non-targeted in the above-mentioned process in a microorganism or plant or a part thereof.

In a further embodiment said polypeptide has the activity of the respective polypeptide represented by a protein comprising a polypeptide as depicted in the respective line in column 5 of Table II.

In a further embodiment the activity of the expression product of one or more nucleic acid molecule(s) comprising a polynucleotide as depicted in the respective line in column 5 or 7 of Table I preferably the coding region thereof, or a homolog or a fragment thereof, is increased or generated non-targeted in the above-mentioned process in a microorganism or plant or a part thereof.

In a further embodiment the activity of the polypeptide comprising a polypeptide as depicted in the respective line in column 5 or 7 of Table II or a homolog or a fragment thereof, a consensus sequence or at least one polypeptide motif as depicted in the respective line in column 7 of Table IV is increased or generated in the above-mentioned process in the cytosol of a cell, of a microorganism or plant.

In a further embodiment said polypeptide has the activity of the respective polypeptide represented by a protein comprising a polypeptide as depicted in the respective line in column 5 of Table II.

In a further embodiment the activity of the expression product of one or more nucleic acid molecule(s) comprising a polynucleotide as depicted in the respective line in column 5 or 7 of Table I preferably the coding region thereof, or a homolog or a fragment thereof, is increased or generated in the above-mentioned process in the cytosol of a cell, of a microorganism or plant.

In a further embodiment of the present invention the process further comprises the step of recovering the fine chemical, which is synthesized by the organism from the organism and/or from the culture medium used for the growth or maintenance of the organism.

For the purposes of the present invention, as a rule the plural is intended to encompass the singular and vice versa, unless otherwise specified.

The terms “increase”, “raise”, “extend”, “enhance”, “improve” and “amplify” as well as the grammatical versions thereof relate to a corresponding change of a property in a non-human organism, a part of an organism such as a tissue, seed, root, leave, flower, pollen etc. or in a cell and are interchangeable. Preferably, the overall activity in the volume is increased or enhanced in cases if the increase or enhancement is related to the increase or enhancement of an activity of a gene product, independent whether the amount of gene product or the specific activity of the gene product or both is increased or enhanced or whether the amount, stability or translation efficacy of the nucleic acid sequence or gene encoding for the gene product is increased or enhanced.

Under “change of a property” it is understood that the activity, expression level or amount of a gene product or the metabolite content is changed in a specific volume relative to a corresponding volume of a control, reference or wild type, including the de novo creation of the activity or expression.

With respect to fine chemicals the term “increase” may be directed to a change of said property in the subject of the present invention or only in a part thereof, for example, the change can be found in a compartment of a cell, like an organelle, or in a part of an non-human organism, like plant tissue, plant seed, plant root, pollen, leave, flower etc. but is not detectable in the overall subject, i.e. complete cell or plant, if tested.

The term “increase” means that the specific activity of a polypeptide or the amount of a compound or of a metabolite, e.g. of a polypeptide, a nucleic acid molecule or an encoding mRNA or DNA or the fine chemical, can be increased in a volume.

The term “increase” includes that a compound or an activity is introduced into a cell or a subcellular compartment or organelle de novo or that the compound or the activity has not been detectable before, in other words it is “generated”. Particularly preferred are increases due the introduction of a DNA, preferably foreign DNA, by recombinant gene technology.

Accordingly, throughout the application, the term “increasing” also comprises the term “generating” or “stimulating”. The increased activity manifests itself in an increase of the fine chemical.

In one embodiment methods of the invention ore performed by overexpression the nucleic acid molecule of the invention in a plant cell or plant.

The invention also includes methods for the production of a product comprising a) growing the plants with increased expression of the DnaJ-like chaperone(s), preferably plants wherein the expression of said DnaJ like chaperone as defined above is increased by biotechnological means e.g. by stable introduction of said DnaJ-like chaperone(s) and b) producing said product from or by the plants of the invention or parts, including seeds, of these plants, wherein the product has an increased content of any one or more fine chemical listed in table FC compared to a product produced from a control plant. In a further embodiment the methods comprise steps a) growing the plants with increased expression of the DnaJ-like chaperone, b) removing the harvestable parts as defined above from the plants and c) producing said product from or by the harvestable parts of the invention, wherein the product has an increased content of any one or more fine chemical listed in table FC compared to a product produced from a control plant.

The product of the inventive processes for the production of said products are superior to the products produced from control plants, since the plant and plant parts used for the production of the product are of improved quality and/or have an increased content of one or more of the fine chemicals listed in table FC. For example, seeds with increased content of the unsaturated fatty acids linoleic and linolenic acid may be such a product, that advantageously can be used in a number of applications ranging from food and feed to the production of oils and lubricants. Biomass with increased sucrose content may be another product of increased property for various applications ranging from the production of sugars, feedstuff, input material for fermentation processes to biological gas or ethanol production.

One example of such inventive methods would be growing corn plants of the invention, harvesting the corn cobs and remove the kernels. These may be used as improved feedstuff or processed to corn starch syrup and oil as agricultural products.

The product may be produced at the site where the plant has been grown, or the plants or parts thereof may be removed from the site where the plants have been grown to produce the product. Typically, the plant is grown, the desired harvestable parts are removed from the plant, if feasible in repeated cycles, and the product made from the harvestable parts of the plant. The step of growing the plant may be performed only once each time the methods of the invention is performed, while allowing repeated times the steps of product production e.g. by repeated removal of harvestable parts of the plants of the invention and if necessary further processing of these parts to arrive at the product. It is also possible that the step of growing the plants of the invention is repeated and plants or harvestable parts are stored until the production of the product is then performed once for the accumulated plants or plant parts. Also, the steps of growing the plants and producing the product may be performed with an overlap in time, even simultaneously to a large extend, or sequentially. Generally the plants are grown for some time before the product is produced.

Advantageously the methods of the invention are more efficient than the known methods, because the plants of the inventive processes have increased yield, yield related trait(s) and stress tolerance to an environmental stress, particularly to limited water availability and drought compared to a control plant used in comparable methods and/or increased content of any one or more fine chemical listed in table FC in the plants, harvestable parts such as seed, shoot biomass or beet biomass and/or products produced. Another embodiment of the present invention is directed to methods for the production of a product with increased content of any one or more fine chemical listed in table FC relative to a product from a control plant comprising the steps of
a. generating one or more plant using any of the inventive methods for increasing content of any one or more fine chemical listed in table FC in plants compared to control plants as described herein,
b. growing the plants of step a.) or progeny plants thereof, i.e. the offspring of plants generated in step a), wherein the progeny plants have increased content, at least in some plant parts used in the methods for the production of said product, of any one or more fine chemical listed in table FC compared to a control plant, and comprise and express, at least in some plant parts, the nucleic acid encoding the DnaJ like chaperone, preferably the recombinant nucleic acid encoding the DnaJ like chaperone, and
c. producing said product from or by
(i) said plants; or
(ii) parts, including seeds, shoot biomass, beet biomass, tubers, of said plants, wherein said plants or parts of said plants have an increased content of any one or more fine chemical listed in table FC relative to a control plant or parts of a control plant.

In one embodiment the products produced by said methods of the invention are plant products such as, but not limited to, a foodstuff, feedstuff, a food supplement, feed supplement, fiber, cosmetic or pharmaceutical. Foodstuffs are regarded as compositions used for nutrition or for supplementing nutrition. Animal feedstuffs and animal feed supplements, in particular, are regarded as foodstuffs.

In another embodiment the inventive methods for the production are used to make agricultural products such as, but not limited to, plant extracts, proteins, amino acids, carbohydrates, fats, oils, polymers, vitamins, and the like.

It is possible that a plant product consists of one ore more agricultural products to a large extent.

In yet another embodiment the polynucleotide sequences or the polypeptide sequences of the invention are comprised in an agricultural product, wherein the agricultural product has an increased content of any one or more fine chemical listed in table FC compared to a agricultural product produced from a control plant.

In a further embodiment the nucleic acid sequences and protein sequences of the invention may be used as product markers, for example for an agricultural product produced by the methods of the invention. Such a marker can be used to identify a product to have been produced by an advantageous process resulting not only in a greater efficiency of the process but also improved quality of the product due to increased quality of the plant material and harvestable parts used in the process. Such markers can be detected by a variety of methods known in the art, for example but not limited to PCR based methods for nucleic acid detection or antibody based methods for protein detection.

The methods of the invention are advantageously applicable to any plant, in particular to any plant as defined herein. Plants that are particularly useful in the methods of the invention include all plants which belong to the superfamily Viridiplantae, in particular monocotyledonous and dicotyledonous plants including fodder or forage legumes, ornamental plants, food crops, trees or shrubs.

According to an embodiment of the present invention, the plant is a crop plant. Examples of crop plants include but are not limited to chicory, carrot, cassaya, trefoil, soybean, beet, sugar beet, sunflower, canola, alfalfa, rapeseed, linseed, cotton, tomato, potato and tobacco.

According to another embodiment of the present invention, the plant is a monocotyledonous plant. Examples of monocotyledonous plants include sugarcane.

According to another embodiment of the present invention, the plant is a cereal. Examples of cereals include rice, maize, wheat, barley, millet, rye, triticale, sorghum, emmer, spelt, einkorn, teff, milo and oats.

In one embodiment the plants used in the methods of the invention are selected from the group consisting of maize, wheat, rice, soybean, cotton, oilseed rape including canola, sugarcane, sugar beet and alfalfa.

In another embodiment of the present invention the plants used in the methods of the invention are sugarcane plants with increased biomass and/or increased sucrose content of the stems.

In another embodiment of the present invention the plants used in the methods of the invention are sugar beet plants with increased biomass and/or increased sucrose content of the beet.

The invention also extends to harvestable parts of a plant such as, but not limited to seeds, leaves, fruits, flowers, stems, roots, rhizomes, beets tubers and bulbs, which harvestable parts comprise a recombinant nucleic acid encoding a DnaJ-like chaperone polypeptide. The invention furthermore relates to products derived or produced, preferably directly derived or directly produced, from a harvestable part of such a plant, such as dry pellets or powders, oil, fat and fatty acids, starch or proteins. In one embodiment the product comprises a recombinant nucleic acid encoding a DnaJ-like chaperone polypeptide and/or a recombinant DnaJ-like chaperone polypeptide.

The present invention also encompasses use of nucleic acids encoding DnaJ-like chaperone polypeptides as described herein and use of these DnaJ-like chaperone polypeptides in enhancing any of the aforementioned yield-related traits in plants under abiotic environmental stress conditions and/or non-stress conditions, preferably under conditions of limited water availability, more preferably under conditions of drought, and/or increased content of any one or more fine chemical listed in table FC relative to control plant. For example, nucleic acids encoding DnaJ-like chaperone polypeptide described herein, or the DnaJ-like chaperone polypeptides themselves, may find use in breeding programmes in which a DNA marker is identified which may be genetically linked to a DnaJ-like chaperone polypeptide-encoding gene. The nucleic acids/genes, or the DnaJ-like chaperone polypeptides themselves may be used to define a molecular marker. This DNA or protein marker may then be used in breeding programmes to select plants having enhanced yield-related traits as defined hereinabove in the methods of the invention. Furthermore, allelic variants of a DnaJ-like chaperone polypeptide-encoding nucleic acid/gene may find use in marker-assisted breeding programmes. Nucleic acids encoding DnaJ-like chaperone polypeptides may also be used as probes for genetically and physically mapping the genes that they are a part of, and as markers for traits linked to those genes. Such information may be useful in plant breeding in order to develop lines with desired phenotypes.

In one embodiment any comparison to determine sequence identity percentages is performed
in the case of a comparison of nucleic acids over the entire coding region of SEQ ID NO: 1 or 41, preferably SEQ ID NO:1, or
in the case of a comparison of polypeptide sequences over the entire length of SEQ ID NO: 2, or 42, preferably SEQ ID NO:12.

For example, a sequence identity of 50% sequence identity in this embodiment means that over the entire coding region of SEQ ID NO: 1, 50 percent of all bases are identical between the sequence of SEQ ID NO: 1 and the related sequence. Similarly, in this embodiment a polypeptide sequence is 50% identical to the polypeptide sequence of SEQ ID NO: 2, when 50 percent of the amino acids residues of the sequence as represented in SEQ ID NO: 2, are found in the polypeptide tested when comparing from the starting methionine to the end of the sequence of SEQ ID NO: 2.

In one embodiment the nucleic acid sequences employed in the methods, constructs, plants, harvestable parts and products of the invention are sequences encoding DnaJ-like chaperone but excluding those nucleic acids encoding the polypeptide sequences disclosed in any of:
1. WO0216655
2. WO2004061 080
3. US2004181830
4. WO03012096
5. EMBL database entry accession no. AK066420

In a further embodiment the nucleic acid sequence employed in methods, constructs, plants, harvestable parts and products of the invention are those sequences that are not the polynucleotides encoding the proteins selected from the group consisting of the proteins of SEQ ID NO: 4, 6, 8, 10, 12, 14, 16, 18, 20, 22, 24, 26, 28, 30, 32, 34, 36, 38, 40 or 42, and those of at least 60, 70, 75, 80, 85, 90, 93, 95, 98 or 99% nucleotide identity when optimally aligned to the sequences encoding the proteins listed in table A, but excluding those coding for the proteins of SEQ ID NO: 4, 6, 8, 10, 12, 14, 16, 18, 20, 22, 24, 26, 28, 30, 32, 34, 36, 38, 40 or 42.

In another embodiment the terms “relative to”, “compared with” and “compared to” may be used interchangeably, preferably when referring to the comparison of plants with control plants, parts or products produced from plants compared to those of control plants or the content of fine chemicals of such.

A further embodiment the terms “expression product” and “gene product” are to be understood as both referring to and being synonymous with DnaJ-like chaperone polypeptide(s) as defined herein above.

In the following, the expression “as defined in claim/item X” is meant to direct the artisan to apply the definition as disclosed in item/claim X. For example, “a nucleic acid as defined in item 1” has to be understood so that the definition of a nucleic acid of item 1 is to be applied to the nucleic acid. In consequence the term “as defined in item” or “as defined in claim” may be replaced with the corresponding definition of that item or claim, respectively.

### Items

The definitions and explanations given herein above apply mutatis mutandis to the following items.
1. A method for increasing content of any one or more fine chemical listed in table FC in plants compared to control plants and for enhancing yield-related traits in plants under stress conditions, preferably under abiotic environmental stress conditions as defined herein, and/or non-stress conditions, comprising modulating expression in a plant of a nucleic acid encoding a POI polypeptide, wherein said POI polypeptide is a DnaJ like chaperone.
2. A method for enhancing yield-related traits in plants under stress conditions, preferably under abiotic environmental stress conditions as defined herein, relative to control plants, comprising modulating expression in a plant of a nucleic acid encoding a POI polypeptide, wherein said POI polypeptide is a DnaJ like chaperone.
3. A method for increasing content of any one or more fine chemical listed in table FC in plants relative to control plants, comprising modulating expression in a plant of a nucleic acid encoding a POI polypeptide, wherein said POI polypeptide is a DnaJ like chaperone.
4. Method according to any one of items 1 to 3, wherein said modulated expression is effected by introducing and expressing in a plant said nucleic acid encoding said POI polypeptide, preferably by introducing and expressing said nucleic acid by biotechnological means as recombinant nucleic acid, preferably by stable integration into the genome of the plant.
5. Method according to any previous item, wherein the nucleic acid encoding the DnaJ-like chaperone is selected from the group consisting of:
   (i) a nucleic acid represented by SEQ ID NO: 1 3, 5, 7, 9, 11, 13, 15, 17, 19, 21, 23, 25, 27, 29, 31, 33, 35, 37, 39 or 41;
   (ii) the complement of a nucleic acid represented by SEQ ID NO: 1 3, 5, 7, 9, 11, 13, 15, 17, 19, 21, 23, 25, 27, 29, 31, 33, 35, 37, 39 or 41;
   (iii) a nucleic acid encoding a POI polypeptide having in increasing order of preference at least 50%, 51%, 62%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence represented by SEQ ID NO: 2, 4, 6, 8, 10, 12, 14, 16, 18, 20, 22, 24, 26, 28, 30, 32, 34, 36, 38, 40 or 42 and additionally comprising one or more domains having in increasing order of preference at least 50%, 55%, 60%, 65%, 70%, 75%, 80%, 85%, 90%, 95%, 96%, 97%, 98%, 99% or 100% sequence identity to any one or more of the PFAM domains PF00226, PF01556 and PF00684, preferably to the conserved domain starting with amino acid 6 up to amino acid 67 and/or to the conserved domain starting with amino acid 143 up to amino acid 208 and/or to the conserved domain starting with amino acid 265 up to amino acid 348 in SEQ ID NO:2, and further preferably conferring enhanced yield-related traits relative to control plants under abiotic environmental stress conditions and/or non-stress conditions, and/or increased fine chemical content of one or more fine chemicals as listed in table FC.
   (iv) a nucleic acid encoding the polypeptide as represented by (any one of) SEQ ID NO: 2, 4, 6, 8, 10, 12, 14, 16, 18, 20, 22, 24, 26, 28, 30, 32, 34, 36, 38, 40 or 42 preferably as a result of the degeneracy of the genetic code, said isolated nucleic acid can be derived or deduced from a polypeptide sequence as represented by (any one of) SEQ ID NO: 2, 4, 6, 8, 10, 12, 14, 16, 18, 20, 22, 24, 26, 28, 30, 32, 34, 36, 38, 40 or 42 and further preferably conferring enhanced yield-related traits relative to control plants under abiotic environmental stress conditions and/or non-stress conditions, and/or increased fine chemical content of one or more fine chemicals as listed in table FC;
   (v) a nucleic acid encoding a POI polypeptide comprising one or more, preferably to all three of the consensus patterns of SEQ ID NO: 45, 46 and 47 and further preferably conferring enhanced yield-related traits relative to control plants under abiotic environmental stress conditions and/or non-stress conditions, and/or increased fine chemical content of one or more fine chemicals as listed in table FC;
   (vi) a nucleic acid molecule which hybridizes with a nucleic acid molecule of (ii) under high stringency hybridization conditions and preferably confers enhanced yield-related traits relative to control plants under abiotic environmental stress conditions and/or non-stress conditions, and/or increased fine chemical content of one or more fine chemicals as listed in table FC.
6. Method according to item any one of items 1, 2, 4 or 5, wherein said enhanced yield-related traits comprise increased (yield—early vigour relative to control plants, and preferably comprise increased biomass and/or increased seed yield relative to control plants.
7. Method according to any one of items 1, 2, 4, 5 or 6, wherein said enhanced yield-related traits are obtained under conditions of drought, salt stress or nitrogen deficiency, preferably drought.
8. Method according to item 1, 2, 4 or 5 wherein said increased content of one or more fine chemical is obtained under non stress conditions.
9. Method according to any of items 1 to 8, wherein said POI polypeptide comprises
a. one or more, preferably two, and more preferably all three of the following PFAM domains PF00226, PF01556 and PF00684 and at least one, preferably any two, more preferably all three of the consensus patterns of SEQ ID NO:45, 46 and 47; and/or
b. a conserved domain starting with amino acid 6 up to amino acid 67 and/or a conserved domain starting with amino acid 143 up to amino acid 208 and/or a conserved domain starting with amino acid 265 up to amino acid 348 in SEQ ID NO:2
10. Method according to any one of items 1 to 9, wherein said nucleic acid molecule or said polypeptide, respectively, is of yeast origin, preferably from the genus Saccharomyces, most preferably from Saccharomyces cerevisiae.
11. Method according to any one of items 1 to 10, wherein said nucleic acid encoding a POI encodes any one of the polypeptides listed in Table II or is a portion of such a nucleic acid, or a nucleic acid capable of hybridising with a complementary sequence of such a nucleic acid.
12. Method according to any one of items 1 to 11, wherein said nucleic acid sequence encodes an orthologue or paralogue of any of the polypeptides given in Table II.
13. Method according to any one of items 1 to 12, wherein said nucleic acid encodes the polypeptide represented by SEQ ID NO: 2 or 42, preferably by SEQ ID NO: 2.
14. Method according to any one of items 1 to 13, wherein said nucleic acid is operably linked to a constitutive promoter.
15. Method according to any of the previous items wherein said plant is a crop plant, preferably a dicot such as sugar beet, alfalfa, trefoil, chicory, carrot, cassaya, cotton, soybean, oilseed rape including canola, or a monocot, such as sugarcane, or a cereal, such as rice, maize, wheat, barley, millet, rye, triticale, sorghum emmer, spelt, secale, einkorn, teff, milo and oats.
16. Use of a construct comprising:
   (i) nucleic acid encoding a POI as defined in any of items 1, 5, 9 to 12;
   (ii) one or more control sequences capable of driving expression of the nucleic acid sequence of (i); and optionally
   (i) a transcription termination sequence.
      for increasing the content of any one or more fine chemical listed in table FC in plants relative to control plants and/or increasing yield-related traits of a plant under stress conditions, preferably under abiotic environmental stress conditions as defined herein, and/or non-stress conditions, preferably under conditions of limited water availability, more preferably under conditions of drought relative to a control plant.
17. Methods according to any of items 1 to 15, wherein the POI encoding nucleic acid is operably linked to a control sequence, or a use according to item 16 wherein one of said control sequences is a constitutive promoter,
18. Harvestable parts of a plant obtainable by a method according to any one of the items 1 to 15, wherein said harvestable part comprises a recombinant nucleic acid encoding said polypeptide as defined in any one of items 1, 5, 9 to 12, wherein said harvestable parts are preferably shoot biomass and/or seeds.
19. Products derived or produced from a plant obtainable by a method according to any one of the items 1 to 15 and/or from harvestable parts of a plant according to item 18.
20. Use of a nucleic acid encoding a POI polypeptide as defined in any of items 1, 5, 9 to 12, for increasing the content of any one or more fine chemical listed in table FC in plants relative to control plants and/or increasing yield-related traits of a plant under stress conditions, preferably under abiotic environmental stress conditions as defined herein, and/or non-stress conditions, preferably under conditions of limited water availability, more preferably under conditions of drought relative to a control plant.
21. A method for the production of a product with increased content of any one or more fine chemical listed in table FC relative to a product from a control plant comprising the steps of
a. generating one or more plants using any of the methods according to any one of items 1 to 15;
b. growing the plants of step a.) or progeny plants thereof, wherein the progeny plants have increased content, at least in some plant parts used in the methods for the production of said product, of any one or more fine chemical listed in table FC compared to a control plant, and comprise and express, at least in some plant parts, the nucleic acid encoding the DnaJ like chaperone, preferably the recombinant nucleic acid encoding the DnaJ like chaperone, and
c. producing said product from or by
(i) said plants; or
(ii) parts, including seeds, shoot biomass, beet biomass, tubers, of said plants, wherein said plants or parts of said plants have an increased content of any one or more fine chemical listed in table FC relative to a control plant or parts of a control plant.
22. Any of the items 1, 3 to 21 wherein the fine chemical increased is sucrose.
23. Any of the items 1, 3 to 21 wherein the fine chemical increased is myo-inositol.
24. Any of the items 1, 3 to 21 wherein the fine chemical increased is linoleic acid.
25. Any of the items 1, 3 to 21 wherein the fine chemical increased is linolenic acid.
26. Any of the items 1, 3 to 21 wherein a combination of any of the fine chemicals sucrose, myo-inositol, linoleic acid and linolenic acid is increased.

### Other Embodiments

### Item A to S:

A. A method for increasing content of any one or more fine chemical listed in table FC in plants compared to control plants and/or for enhancing yield in plants under stress conditions, preferably under abiotic environmental stress conditions as defined herein, and/or non-stress conditions, preferably under conditions of limited water availability, more preferably under conditions of drought, comprising modulating expression in a plant of a nucleic acid molecule encoding a polypeptide, wherein said polypeptide is a DnaJ like chaperone
B. Method according to item A, wherein said polypeptide comprises
   a. one or more, preferably two and more preferably all three of the following PFAM domains PF00226, PF01556 and PF00684 and at least one, preferably any two, more preferably all three of the consensus patterns of SEQ ID NO:45, 46 and 47; and/or
   b. the conserved domain starting with amino acid 6 up to amino acid 67 and/or to the conserved domain starting with amino acid 143 up to amino acid 208 and/or to the conserved domain starting with amino acid 265 up to amino acid 348 in SEQ ID NO:2.
C. Method according to item A or B, wherein said modulated expression is effected by introducing and expressing in a plant a nucleic acid molecule encoding a DnaJ-like chaperone, preferably by introducing and expressing said nucleic acid by biotechnological means as recombinant nucleic acid, preferably by stable integration into the genome of the plant.
D. Method according to any one of items A to C, wherein said polypeptide is encoded by a nucleic acid molecule comprising a nucleic acid molecule selected from the group consisting of:
   (i) a nucleic acid represented by (any one of) SEQ ID NO: 1 3, 5, 7, 9, 11, 13, 15, 17, 19, 21, 23, 25, 27, 29, 31, 33, 35, 37, 39 or 41;
   (ii) the complement of a nucleic acid represented by (any one of) SEQ ID NO: 1 3, 5, 7, 9, 11, 13, 15, 17, 19, 21, 23, 25, 27, 29, 31, 33, 35, 37, 39 or 41;
   (iii) a nucleic acid encoding the polypeptide as represented by (any one of) SEQ ID NO: 2, 4, 6, 8, 10, 12, 14, 16, 18, 20, 22, 24, 26, 28, 30, 32, 34, 36, 38, 40 or 42 preferably as a result of the degeneracy of the genetic code, said isolated nucleic acid can be deduced from a polypeptide sequence as represented by (any one of) SEQ ID NO: 2, 4, 6, 8, 10, 12, 14, 16, 18, 20, 22, 24, 26, 28, 30, 32, 34, 36, 38, 40 or 42 and further preferably conferring enhanced yield-related traits relative to control plants under abiotic environmental stress conditions and/or non-stress conditions, and/or increased fine chemical content of one or more fine chemicals as listed in table FC;
   (iv) a nucleic acid having, in increasing order of preference at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with any of the nucleic acid sequences of SEQ ID NO: 1 3, 5, 7, 9, 11, 13, 15, 17, 19, 21, 23, 25, 27, 29, 31, 33, 35, 37, 39 or 41, and further preferably conferring enhanced yield-related traits relative to control plants under abiotic environmental stress conditions and/or non-stress conditions, and/or increased fine chemical content of one or more fine chemicals as listed in table FC
   (v) a first nucleic acid molecule which hybridizes with a second nucleic acid molecule of (i) to (iv) under stringent hybridization conditions and further preferably conferring enhanced yield-related traits relative to control plants under abiotic environmental stress conditions and/or non-stress conditions, and/or increased fine chemical content of one or more fine chemicals as listed in table FC;
   (vi) a nucleic acid encoding said polypeptide having, in increasing order of preference, at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence represented by (any one of) SEQ ID NO: 2, 4, 6, 8, 10, 12, 14, 16, 18, 20, 22, 24, 26, 28, 30, 32, 34, 36, 38, 40 or 42 and further preferably conferring enhanced yield-related traits relative to control plants under abiotic environmental stress conditions and/or non-stress conditions, and/or increased fine chemical content of one or more fine chemicals as listed in table FC; or
   (vii) a nucleic acid comprising any combination(s) of features of (i) to (vi) above.
E. Method according to any item A to D, wherein said enhanced yield-related traits comprise increased yield, preferably seed yield and/or shoot biomass relative to control plants.
F. Method according to any one of items A to E, wherein said enhanced yield-related traits are obtained under conditions of limited water availability.
G. Method according to any one of items A to E, wherein said enhanced yield-related traits are obtained under conditions of drought stress, salt stress or nitrogen deficiency.
H. Method according to any one of items A to D wherein the increased in at least one fine chemical is obtained under non-stress conditions.
I. Method according to any one of items A to D, F or G wherein the increased in at least one fine chemical is obtained under abiotic environmental stress conditions, preferably conditions of limited water availability, more preferably under drought stress conditions.
J. Method according to any one of items A to I, wherein said nucleic acid is operably linked to a constitutive promoter, preferably to a Big35S promoter.
K. Method according to any one of items A to J, wherein said nucleic acid molecule or said polypeptide, respectively, is of plant origin, preferably from a monocot plant, further preferably from the family Poaceae, more preferably from the genus Oryza, most preferably from rice.
L. Method according to any one of items A to J, wherein said nucleic acid molecule or said polypeptide, respectively, is of yeast origin, preferably from the genus Saccharomyces, most preferably from Saccharomyces cerevisiae.
M. Use of a construct comprising:
(i) nucleic acid encoding said polypeptide as defined in any one of items A to D, K or L;
(ii) one or more control sequences capable of driving expression of the nucleic acid sequence of (a); and optionally
(iii) a transcription termination sequence;
   in a method for increasing the content of any one or more fine chemical listed in table FC in plants relative to control plants and/or increasing yield-related traits of a plant under stress conditions, preferably under abiotic environmental stress conditions as defined herein, and/or non-stress conditions, preferably under conditions of limited water availability, more preferably under conditions of drought relative to a control plant.
N. A method for the production of a product with increased content of content of any one or more fine chemical listed in table FC relative to a product from a control plant comprising the steps of
   i. generating one or more plants using any of the methods according to any one of items A to L;
   ii. growing the plants of step a.) or progeny plants thereof, wherein the progeny plants have increased content, at least in some plant parts used in the methods for the production of said product, of any one or more fine chemical listed in table FC compared to a control plant, and comprise and express, at least in some plant parts, the nucleic acid encoding the DnaJ like chaperone, preferably the recombinant nucleic acid encoding the DnaJ like chaperone, and
   c. producing said product from or by
      (i) said plants; or
      (ii) parts, including seeds, shoot biomass, beet biomass, tubers, of said plants,
      wherein said plants or parts of said plants have an increased content of any one or more fine chemical listed in table FC relative to a control plant or parts of a control plant.
O. Method of any item A to L or N wherein said plant is a crop plant, preferably a dicot such as sugar beet, alfalfa, trefoil, chicory, carrot, cassaya, cotton, soybean, canola or a monocot, such as sugarcane, or a cereal, such as rice, maize, wheat, barley, millet, rye, triticale, sorghum emmer, spelt, secale, einkorn, teff, milo and oats.
P. Harvestable parts of a plant obtainable by a method according to any one of items A to L or O, wherein said harvestable part thereof comprises a recombinant nucleic acid encoding said polypeptide as defined in any one of items A to D, J, K, or L, wherein said harvestable parts are preferably shoot and/or root biomass and/or seeds.
Q. Products produced from a plant obtainable by a method according to any one of items A to L or O and/or from harvestable parts of a plant according to item P.
R. Use of a nucleic acid encoding a polypeptide as defined in any one of items A to D, K, L for increasing the content of any one or more fine chemical listed in table FC in plants relative to control plants and/or increasing yield-related traits of a plant under stress conditions, preferably under abiotic environmental stress conditions as defined herein, and/or non-stress conditions, preferably under conditions of limited water availability, more preferably under conditions of drought relative to a control plant.

### DESCRIPTION OF FIGURES

The present invention will now be described with reference to the following figures in which:

FIG. 1 Vector pMTX155 (SEQ ID NO: 48) used for used for cloning gene of interest for non-targeted expression.

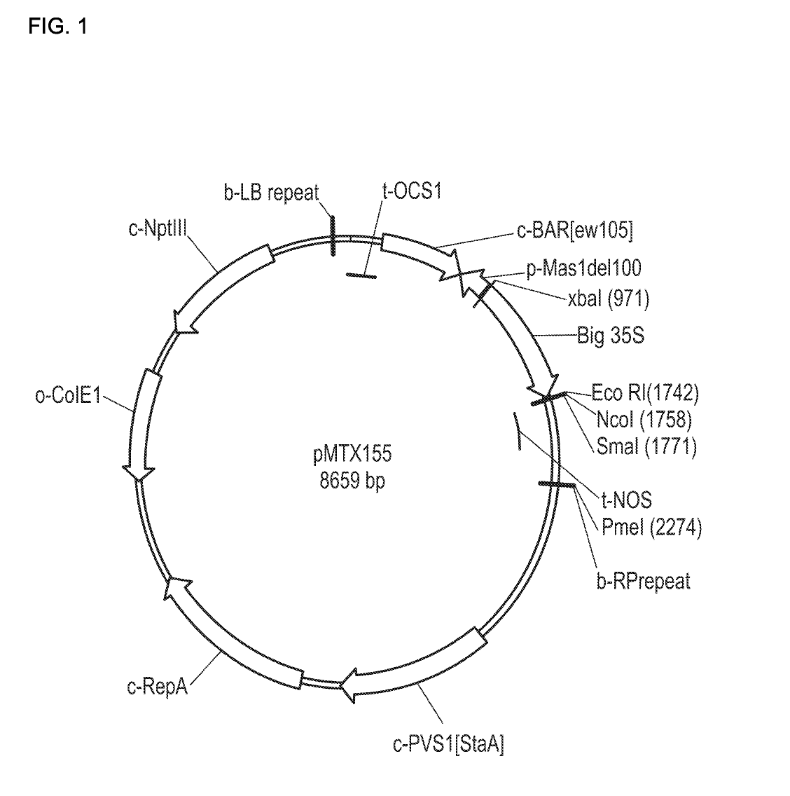

### TABLES 0 TO III

In a line of Table I related nucleic acid molecules are listed. In column 3 the locus name, often also referred to as gene name, is given, in column 5 the lead sequence ID No. thereto and in column 7 the sequence ID No. of homologues thereof. In the corresponding line of Table II the respective polypeptides are listed. In column 3 the protein name is given (which is according to the common understanding of the skilled person in the art usually used for the gene as well as the polypeptide and therefore identical with the gene name/locus name), in column 5 the (corresponding) lead sequence ID No. thereto and in column 7 the (corresponding) sequence ID No. of homologues thereof.

In Tables I and II in column 4 information is given from which organism the lead sequence according to column 5 has been identified, in column 7 information is given which fine chemical is generated or increased, and in an especial embodiment in column 6 information is given about non-targeted expression or expression in plastids or mitochondria.

Tables III and IV are arranged accordingly whereby in column 7 of Table III primers are listed which can be used to amplify the sequence of the corresponding lead sequence indicated in column 5 of the same line and whereby in column 7 of Table IV consensus and pattern sequences are listed which are shared by the lead sequence as indicated in column 5 of the same line and their homologs listed in the same line in Table II column 7. How the consensus and pattern sequences are determined is described later on in the application in more detail.

Table 0 showing binary vectors used in Example 8

Overview of the different vectors used for cloning the ORFs; showing their SEQ ID NOs (column 1), their vector names (column 2), the promoters they contain for expression of the ORFs (column 3), if present, the additional artificial targeting sequence (column 4), the adapter sequence

(column 5), the expression type conferred by the promoter mentioned in column 3 (column 6) and the figure number (column 7).

| | Vector | Promoter | Target | Adapter | | |
|---|---|---|---|---|---|---|
| SeqID | Name | Name | Sequence | Sequence | Expression Type | FIG. |
| 48 | pMTX155 | Big35S | | Resgen | non targeted constitutive expression | 5 |
| | | | | | preferentially in green tissues | |

In column 3 PcUbi refers to the PcUbi promoter (Kawalleck et al., Plant. Molecular Biology, 21, 673 (1993)) also named p-PcUBI in table d, Super to the Super promoter (Ni et al., Plant Journal 7, 661 (1995), WO 95/14098) also named p-Super in table d, Big35S to the enhanced 35S promoter (Comai et al., Plant Mol Biol 15, 373-383 (1990) and USP to the USP promoter (Baeumlein et al., Mol Gen Genet. 225(3):459-67 (1991)) als named p-USP in table d.

TABLE I
Nucleic acid sequence ID numbers

| | | | | | 5. | | |
|---|---|---|---|---|---|---|---|
| | | | | | Lead | | |
| | 1. | 2. | 3. | 4. | SEQ | 6. | 7. |
| [symbol] pplication | Hit | Project | Locus | [symbol] rganism | ID | Target | SEQ IDs of Nucleic Acid Homologs |
| 1 | 1 | YNL064C_11 | YNL064C | S. cerevisiae | 1 | cytoplasmic | 3, 5, 7, 9, 11, 13, 15, 17, 19, 21, 23, 25, 27, 29, |
| | | | | | | | 31, 33, 35, 37, 39, 41, |

[symbol]

indicates data missing or illegible when filed

TABLE II
Amino acid sequence ID numbers

| | | | | | 5. | | |
|---|---|---|---|---|---|---|---|
| | | | | | Lead | | |
| | 1. | 2. | 3. | 4. | SEQ | 6. | 7. |
| [symbol] pplication | Hit | Project | Locus | Organism | ID | Target | SEQ IDs of Polypeptide Homologs |
| 1 | 1 | YNL064C_11 | YNL064C | S. cerevisiae | 2 | cytoplasmic | 4, 6, 8, 10, 12, 14, 16, 18, 20, 22, 24, 26, |
| | | | | | | | 28, 30, 32, 34, 36, 38, 40, 42 |

[symbol]

indicates data missing or illegible when filed

TABLE III
Primer nucleic acid sequence ID numbers

| | | | | | 5. | | |
|---|---|---|---|---|---|---|---|
| | | | | | Lead | | |
| | 1. | 2. | 3. | 4. | SEQ | 6. | 7. |
| [symbol] pplication | Hit | Project | Locus | Organism | ID | Target | SEQ IDs of Primers |
| 1 | 1 | YNL064C_11 | YNL064C | S. cerevisiae | 1 | cytoplasmic | 43, 44 |

[symbol]

indicates data missing or illegible when filed

TABLE IV
Consensus amino acid sequence ID numbers

| | | | | | 5. | | |
|---|---|---|---|---|---|---|---|
| | | | | | Lead | | 7. |
| | 1. | 2. | 3. | 4. | SEQ | 6. | SEQ IDs of Consensus/Pattern |
| Application | Hit | Project | Locus | Organism | ID | Target | Sequences |
| 1 | 1 | YNL064C_11 | YNL064C | S. cerevisiae | 2 | cytoplasmic | 45, 46, 47 |

### EXAMPLES

The present invention will now be described with reference to the following examples, which are by way of illustration only. The following examples are not intended to limit the scope of the invention.

DNA manipulation: unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

### Example 1

### Identification of sequences related to SEQ ID NO: 1 and SEQ ID NO: 2

Sequences (full length cDNA, ESTs or genomic) related to SEQ ID NO: 1 and SEQ ID NO: 2 were identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. For example, the polypeptide encoded by the nucleic acid of SEQ ID NO: 1 was used for the TBLASTN algorithm, with default settings and the filter to ignore low complexity sequences set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search. For example the E-value may be increased to show less stringent matches. This way, short nearly exact matches may be identified.

Table I provides a list of nucleic acid sequences related to SEQ ID NO: 1 and table II a list of amino acid sequences related to SEQ ID NO: 2.

Sequences have been tentatively assembled and publicly disclosed by research institutions, such as The Institute for Genomic Research (TIGR; beginning with TA). For instance, the Eukaryotic Gene Orthologs (EGO) database may be used to identify such related sequences, either by keyword search or by using the BLAST algorithm with the nucleic acid sequence or polypeptide sequence of interest. Special nucleic acid sequence databases have been created for particular organisms, e.g. for certain prokaryotic organisms, such as by the Joint Genome Institute. Furthermore, access to proprietary databases, has allowed the identification of novel nucleic acid and polypeptide sequences.

### Example 2

### Alignment of DnaJ-Like Chaperone Polypeptide Sequences

Alignment of polypeptide sequences is performed using the ClustalW 2.0 algorithm of progressive alignment (Thompson et al. (1997) Nucleic Acids Res 25:4876-4882; Chema et al. (2003). Nucleic Acids Res 31:3497-3500) with standard setting (slow alignment, similarity matrix: Gonnet, gap opening penalty 10, gap extension penalty: 0.2). Minor manual editing is done to further optimise the alignment.

A phylogenetic tree of DnaJ-like chaperone polypeptides is constructed by aligning DnaJ-like chaperone sequences using MAFFT (Katoh and Toh (2008)—Briefings in Bioinformatics 9:286-298). A neighbour-joining tree was calculated using Quick-Tree (Howe et al. (2002), Bioinformatics 18(11): 1546-7), 100 bootstrap repetitions. The dendrogram is drawn using Dendroscope (Huson et al. (2007), BMC Bioinformatics 8(1):460). Confidence levels for 100 bootstrap repetitions are indicated for major branchings.

### Example 3

### Calculation of Global Percentage Identity Between Polypeptide Sequences

Global percentages of similarity and identity between full length polypeptide sequences useful in performing the methods of the invention is determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix.

### Example 4

### Identification of Domains Comprised in Polypeptide Sequences Useful in Performing the Methods of the Invention

### Pfam Domain Search

For identification of protein domains as defined in the Pfam Protein Families Database, protein sequences were searched using the hmmscan algorithm. hmmscan is part of the HMMER3 software package that is public available from the Howard Hughes Medical Institute, Janelia Farm Research Campus (http://hmmer.org/). Search for Pfam domains was done using release 25.0 (released March 2011) of the Pfam Protein Families Database (http://pfam.sanger.ac.uk/). Parameters for hmmscan algorithm were the default parameters implemented in hmmscan (HMMER release 3.0). Domains reported by the hmmscan algorithm were taken into account if the independent E-value was 0.1 or better and if at least 80% of the PFAM domain model length was covered by the alignment.

### Annotation of Identified Pfam Domain

### Domain 1: DnaJ (PF00226)

Hsp40 (heat shock protein 40 kD) also known as DnaJ is a family of heat shock proteins that are expressed in a wide variety of organisms from bacteria to humans.

Hsp40 is a family of heat-shock proteins that contain a 70 amino-acid consensus sequence known as the J domain. The J domain of Hsp40 interacts with Hsp70 heat shock proteins. Hsp40 heat-shock proteins play a role in regulating the ATPase activity of Hsp70 heat-shock proteins (Reference: http://pfam.sanger.ac.uk).

### Domain 2: DnaJ_C (PF01556) (DnaJ_C=DnaJ C Terminal Domain)

This family consists of the C terminal region form the DnaJ protein. Although the function of this region is unknown, it is often found associated with PF00226 and PF00684. DnaJ is a chaperone associated with the Hsp70 heat-shock system involved in protein folding and renaturation after stress (Reference: http://pfam.sanger.ac.uk)

### Domain 3: DnaJ_CXXCXGXG (PF00684) DnaJ Central Domain

The central cysteine-rich (CR) domain of DnaJ proteins contains four repeats of the motif CXXCXGXG where X is any amino acid. The isolated cysteine rich domain folds in zinc dependent fashion. Each set of two repeats binds one unit of zinc. Although this domain has been implicated in substrate binding, no evidence of specific interaction between the isolated DNAJ cysteine rich domain and various hydrophobic peptides has been found (Reference: http://pfam.sanger.ac.uk)

### Interpro

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, Propom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom.

In an embodiment a DnaJ-like chaperone polypeptide comprises a conserved domain (or motif) with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a conserved domain from amino acid 6 up to amino acid 67 and/or to the conserved domain starting with amino acid 143 up to amino acid 208 and/or to the conserved domain starting with amino acid 265 up to amino acid 348 in SEQ ID NO:2.

### Example 5

### Topology Prediction of the DnaJ-Like Chaperone Polypeptide Sequences

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark
PSORT (URL: psort.org)
PLOC (Park and Kanehisa, Bioinformatics, 19, 1656-1663, 2003).

### Example 6

### Identification of Identical and Heterologous Genes

Gene sequences can be used to identify identical or heterologous genes from cDNA or genomic libraries. Identical genes (e.g. full-length cDNA clones) can be isolated via nucleic acid hybridization using for example cDNA libraries. Depending on the abundance of the gene of interest, 100,000 up to 1,000,000 recombinant bacteriophages are plated and transferred to nylon membranes. After denaturation with alkali, DNA is immobilized on the membrane by e.g. UV cross linking. Hybridization is carried out at high stringency conditions. In aqueous solution, hybridization and washing is performed at an ionic strength of 1 M NaCl and a temperature of 68° C. Hybridization probes are generated by e.g. radioactive (32P) nick transcription labeling (High Prime, Roche, Mannheim, Germany). Signals are detected by autoradiography.

Partially identical or heterologous genes that are related but not identical can be identified in a manner analogous to the above-described procedure using low stringency hybridization and washing conditions. For aqueous hybridization, the ionic strength is normally kept at 1 M NaCl while the temperature is progressively lowered from 68 to 42° C.

Isolation of gene sequences with homology (or sequence identity/similarity) only in a distinct domain of (for example 10-20 amino acids) can be carried out by using synthetic radio labeled oligonucleotide probes. Radiolabeled oligonucleotides are prepared by phosphorylation of the 5-prime end of two complementary oligonucleotides with T4 polynucleotide kinase. The complementary oligonucleotides are annealed and ligated to form concatemers. The double stranded concatemers are than radiolabeled by, for example, nick transcription. Hybridization is normally performed at low stringency conditions using high oligonucleotide concentrations.

### Oligonucleotide Hybridization Solution:

### 6×SSC

0.01 M sodium phosphate

### 1 mM EDTA (pH 8)

### 0.5% SDS

100 μg/ml denatured salmon sperm DNA
0.1% nonfat dried milk

During hybridization, temperature is lowered stepwise to 5-10° C. below the estimated oligonucleotide Tm or down to room temperature followed by washing steps and autoradiography. Washing is performed with low stringency such as 3 washing steps using 4×SSC. Further de-tails are described by Sambrook J. et al., 1989, “Molecular Cloning: A Laboratory Manual,” Cold Spring Harbor Laboratory Press or Ausubel F. M. et al., 1994, “Current Protocols in Molecular Biology,” John Wiley & Sons.

### Example 7

### Identification of Identical Genes by Screening Expression Libraries with Antibodies

c-DNA clones can be used to produce recombinant polypeptide for example in E. coli (e.g. Qiagen QIAexpress pQE system). Recombinant polypeptides are then normally affinity purified via Ni-NTA affinity chromatography (Qiagen). Recombinant polypeptides are then used to produce specific antibodies for example by using standard techniques for rabbit immunization. Antibodies are affinity purified using a Ni-NTA column saturated with the recombinant antigen as described by Gu et al., BioTechniques 17, 257 (1994). The antibody can than be used to screen expression cDNA libraries to identify identical or heterologous genes via an immunological screening (Sambrook J., et al., “Molecular Cloning: A Laboratory Manual” Cold Spring Harbor Laboratory Press, 1989, or Ausubel F. M. et al., “Current Protocols in Molecular Biology”, John Wiley & Sons, 1994).

### Example 8

### Cloning of the DnaJ-like chaperone encoding nucleic acid sequence

### Example 8a

### PCR Amplification of the Sequences

Unless otherwise specified, standard methods as described in Sambrook et al., Molecular Cloning: A laboratory manual, Cold Spring Harbor 1989, Cold Spring Harbor Laboratory Press are used.

The inventive sequences as shown in the respective line in Table I, column 5, preferably the coding region thereof, were amplified by PCR as described in the protocol of the Pfu Ultra, Pfu Turbo or Herculase DNA polymerase (Stratagene). The composition for the protocol of the Pfu Ultra, Pfu Turbo or Herculase DNA polymerase was as follows: 1×PCR buffer (Stratagene), 0.2 mM of each dNTP, 100 ng genomic DNA of Saccharomyces cerevisiae (strain S288C; Research Genetics, Inc., now Invitrogen), Escherichia coli (strain MG1655; E. coli Genetic Stock Center), Synechocystis sp. (strain PCC6803), Azotobacter vinelandii (strain N. R. Smith, 16), Thermus thermophilus (HB8) or 50 ng cDNA from various tissues and development stages of Arabidopsis thaliana (ecotype Columbia), Physcomitrella patens, Glycine max (variety Resnick), Brassica napus, Oryza sativa or Zea mays (variety B73, Mo17, A188), 50 μmol forward primer, 50 μmol reverse primer, with or without 1 M Betaine, 2.5 u Pfu Ultra, Pfu Turbo or Herculase DNA polymerase.

The amplification cycles were as follows:

1 cycle of 2-3 minutes at 94-95° C., then 25-36 cycles with 30-60 seconds at 94-95° C., 30-45 seconds at 50-60° C. and 210-480 seconds at 72° C., followed by 1 cycle of 5-10 minutes at 72° C., then 4-16° C.—preferably for Saccharomyces cerevisiae, Escherichia coli, Synechocystis sp., Azotobacter vinelandii, Thermus thermophilus.

In case of Arabidopsis thaliana, Brassica napus, Glycine max, Oryza sativa, Physcomitrella patens, Zea mays the amplification cycles are as follows:

1 cycle with 30 seconds at 94° C., 30 seconds at 61° C., 15 minutes at 72° C., then 2 cycles with 30 seconds at 94° C., 30 seconds at 60° C., 15 minutes at 72° C., then 3 cycles with 30 seconds at 94° C., 30 seconds at 59° C., 15 minutes at 72° C., then 4 cycles with 30 seconds at 94° C., 30 seconds at 58° C., 15 minutes at 72° C., then 25 cycles with 30 seconds at 94° C., 30 seconds at 57° C., 15 minutes at 72° C., then 1 cycle with 10 minutes at 72° C., then finally 4-16° C.

RNAs were generated with the RNeasy Plant Kit according to the standard protocol (Qiagen) and Superscript II Reverse Transkriptase was used to produce double stranded cDNA according to the standard protocol (Invitrogen).

ORF specific primer pairs for the genes to be expressed are shown in the respective line in Table III, column 7. The following adapter sequences were added to Saccharomyces cerevisiae ORF specific primers (see Table III) for cloning purposes:

| | | |
|---|---|---|
| i) | foward primer: | 5′-GGAATTCCAGCTGACCACC-3′ |
| ii) | reverse primer: | 5′-GATCCCCGGGAATTGCCATG-3′ |

These adaptor sequences allow cloning of the ORF into the various vectors containing the Resgen adaptors, see table column 5 of Table 0.

The following adapter sequences may be added to Saccharomyces cerevisiae, Escherichia coli, Synechocystis sp., Azotobacter vinelandii, Thermus thermophilus, Arabidopsis thaliana, Brassica napus, Glycine max, Oryza sativa, Physcomitrella patens, or Zea mays ORF specific primers for cloning purposes:

| | | |
|---|---|---|
| iii) | forward primer: | 5′-TTGCTCTTCC- 3′ |
| iiii) | reverse primer: | 5′-TTGCTCTTCG-3′ |

The adaptor sequences allow cloning of the ORF into the various vectors containing the Colic adaptors.

Therefore for amplification and cloning of Saccharomyces cerevisiae SEQ ID NO: 1, a primer consisting of the adaptor sequence i) and the ORF specific sequence SEQ ID NO: 43 and a second primer consisting of the adaptor sequence ii) and the ORF specific sequence SEQ ID NO: 44 were used.

For amplification and cloning of Saccharomyces cerevisiae, Escherichia coli, Synechocystis sp., Azotobacter vinelandii, Thermus thermophilus, Arabidopsis thaliana, Brassica napus, Glycine max, Oryza sativa, Physcomitrella patens, or Zea mays, a primer consisting of the adaptor sequence iii) and an ORF specific sequence A and a second primer consisting of the adaptor sequence iiii) and a second ORF specific sequence B are used.

Following these examples every sequence disclosed in Table I, preferably column 5, especially the coding region thereof can be cloned by fusing the adaptor sequences to the respective specific primers sequences as disclosed in Table III, column 7 using the vector shown in Table 0 or other vectors known in the art.

The DNA is sequenced by standard procedures, in particular the chain determination method, using ABI377 sequencers (see, for example, Fleischman R. D. et al., Science 269, 496 (1995)).

### Example 8b

### Construction of Binary Vectors for Non-Targeted Expression of Proteins

“Non-targeted” expression in this context means, that no additional targeting sequences were added to the ORF to be expressed.

For non-targeted expression the binary vector used for cloning was pMTX155 (SEQ ID NO:48), VC-MME220-1qcz, VC-MME221-1qcz, and VC-MME489-1QCZ. Other useful binary vectors are known to the skilled worker; an overview of binary vectors and their use can be found in Hellens R., Mullineaux P. and Klee H. (Trends in Plant Science, 5 (10), 446 (2000)). Such vectors have to be equally equipped with appropriate promoters and targeting sequences.

### Example 8c

### Cloning of Inventive Sequences as Shown in Table I, Column 5 in the Different Expression Vectors

For cloning for example the ORFs of SEQ ID NO: 1 from Saccharomyces cerevisiae or any other ORF from Saccharomyces cerevisiae into vectors containing the Resgen adaptor sequence the respective vector DNA was treated with the restriction enzyme NcoI.

The reaction was stopped by inactivation at 70° C. for 20 minutes and purified over QIAquick or NucleoSpin Extract II columns following the standard protocol (Qiagen or Macherey-Nagel).

Then the PCR-product representing the amplified ORF with the respective adapter sequences and the vector DNA were treated with T4 DNA polymerase according to the standard protocol (MBI Fermentas) to produce single stranded overhangs with the parameters 1 unit T4 DNA polymerase at 37° C. for 2-10 minutes for the vector and 1-2 u T4 DNA polymerase at 15-17° C. for 10-60 minutes for the PCR product representing SEQ ID NO: 7081.

The reaction was stopped by addition of high-salt buffer and purified over QIAquick or Nucleo-Spin Extract II columns following the standard protocol (Qiagen or Macherey-Nagel).

According to this example the skilled person is able to clone all sequences disclosed in Table I, preferably column 5 or column 7, especially the coding region thereof. Approximately 30-60 ng of prepared vector and a defined amount of prepared amplificate were mixed and hybridized at 65° C. for 15 minutes followed by 37° C. 0.1° C./1 seconds, followed by 37° C. 10 minutes, followed by 0.1° C./1 seconds, then 4-10° C. The ligated constructs were transformed in the same reaction vessel by addition of competent E. coli cells (strain DHSalpha) and incubation for 20 minutes at 1° C. followed by a heat shock for 90 seconds at 42° C. and cooling to 1-4° C. Then, complete medium (SOC) was added and the mixture was incubated for 45 minutes at 37° C. The entire mixture was subsequently plated onto an agar plate with 0.05 mg/ml kanamycin and incubated overnight at 37° C.

The outcome of the cloning step was verified by amplification with the aid of primers which bind upstream and downstream of the integration site, thus allowing the amplification of the insertion. The amplifications were carried out as described in the protocol of Taq DNA polymerase (Gibco-BRL).

The amplification cycles were as follows:

1 cycle of 1-5 minutes at 94° C., followed by 35 cycles of in each case 15-60 seconds at 94° C., 15-60 seconds at 50-66° C. and 5-15 minutes at 72° C., followed by 1 cycle of 10 minutes at 72° C., then 4-16° C.

Several colonies were checked, but only one colony for which a PCR product of the expected size was detected was used in the following steps.

A portion of this positive colony was transferred into a reaction vessel filled with complete medium (LB) supplemented with kanamycin and incubated overnight at 37° C.

The plasmid preparation was carried out as specified in the Qiaprep or NucleoSpin Multi-96 Plus standard protocol (Qiagen or Macherey-Nagel).

### Example 9

### Generation of Transgenic Arabidopsis Plants which Express SEQ ID NO: 1

1-5 ng of the plasmid DNA isolated was transformed by electroporation or transformation into competent cells of Agrobacterium tumefaciens, of strain GV 3101 pMP90 (Koncz and Schell, Mol. Gen. Gent. 204, 383 (1986)). Thereafter, complete medium (YEP) was added and the mix-ture was transferred into a fresh reaction vessel for 3 hours at 28° C. Thereafter, all of the reac-tion mixture was plated onto YEP agar plates supplemented with the respective antibiotics, e.g. rifampicine (0.1 mg/ml), gentamycine (0.025 mg/ml and kanamycin (0.05 mg/ml) and incubated for 48 hours at 28° C.

The agrobacteria that contains the plasmid construct were then used for the transformation of plants.

A colony was picked from the agar plate with the aid of a pipette tip and taken up in 3 ml of liquid TB medium, which also contained suitable antibiotics as described above. The preculture was grown for 48 hours at 28° C. and 120 rpm.

400 ml of LB medium containing the same antibiotics as above were used for the main culture. The preculture was transferred into the main culture. It was grown for 18 hours at 28° C. and 120 rpm. After centrifugation at 4 000 rpm, the pellet was resuspended in infiltration medium (MS medium, 10% sucrose).

In order to grow the plants for the transformation, dishes (Piki Saat 80, green, provided with a screen bottom, 30×20×4.5 cm, from Wiesauplast, Kunststofftechnik, Germany) were half-filled with a GS 90 substrate (standard soil, Werkverband E.V., Germany). The dishes were watered overnight with 0.05% Proplant solution (Chimac-Apriphar, Belgium). A. thaliana C24 seeds (Nottingham Arabidopsis Stock Centre, UK; NASC Stock N906) were scattered over the dish, ap-proximately 1 000 seeds per dish. The dishes were covered with a hood and placed in the stratification facility (8 h, 110 μmol m-2 s-1, 22° C.; 16 h, dark, 6° C.). After 5 days, the dishes were placed into the short-day controlled environment chamber (8 h, 130 μmol m-2 s-1, 22° C.; 16 h, dark, 20° C.), where they remained for approximately 10 days until the first true leaves had formed.

The seedlings were transferred into pots containing the same substrate (Teku pots, 7 cm, LC series, manufactured by Poppelmann GmbH & Co, Germany). Five plants were pricked out into each pot. The pots were then returned into the short-day controlled environment chamber for the plant to continue growing.

After 10 days, the plants were transferred into the greenhouse cabinet (supplementary illumination, 16 h, 340 μmol m-2 s-1, 22° C.; 8 h, dark, 20° C.), where they were allowed to grow for further 17 days.

For the transformation, 6-week-old Arabidopsis plants, which had just started flowering were immersed for 10 seconds into the above-described agrobacterial suspension which had previously been treated with 10 μl Silwett L77 (Crompton S. A., Osi Specialties, Switzerland). The method in question is described by Clough J. C. and Bent A. F. (Plant J. 16, 735 (1998)).

The plants were subsequently placed for 18 hours into a humid chamber. Thereafter, the pots were returned to the greenhouse for the plants to continue growing. The plants remained in the greenhouse for another 10 weeks until the seeds were ready for harvesting.

Depending on the tolerance marker used for the selection of the transformed plants the har-vested seeds were planted in the greenhouse and subjected to a spray selection or else first sterilized and then grown on agar plates supplemented with the respective selection agent. Since the vector contained the bar gene as the tolerance marker, plantlets were sprayed four times at an interval of 2 to 3 days with 0.02% BASTA® and transformed plants were allowed to set seeds.

The seeds of the transgenic A. thaliana plants were stored in the freezer (at −20° C.).

### Example 10

### Transformation of Other Plants

### Rice Transformation

The Agrobacterium containing the expression vector is used to transform Oryza sativa plants. Mature dry seeds of the rice japonica cultivar Nipponbare are dehusked. Sterilization is carried out by incubating for one minute in 70% ethanol, followed by 30 minutes in 0.2% HgCl₂, followed by a 6 times 15 minutes wash with sterile distilled water. The sterile seeds are then germinated on a medium containing 2,4-D (callus induction medium). After incubation in the dark for four weeks, embryogenic, scutellum-derived calli are excised and propagated on the same medium. After two weeks, the calli are multiplied or propagated by subculture on the same medium for another 2 weeks. Embryogenic callus pieces are sub-cultured on fresh medium 3 days before co-cultivation (to boost cell division activity).

Agrobacterium strain LBA4404 containing the expression vector is used for co-cultivation. Agrobacterium is inoculated on AB medium with the appropriate antibiotics and cultured for 3 days at 28° C. The bacteria are then collected and suspended in liquid co-cultivation medium to a density (OD₆₀₀) of about 1. The suspension is then transferred to a Petri dish and the calli immersed in the suspension for 15 minutes. The callus tissues are then blotted dry on a filter paper and transferred to solidified, co-cultivation medium and incubated for 3 days in the dark at 25° C. Co-cultivated calli are grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential is released and shoots developed in the next four to five weeks. Shoots are excised from the calli and incubated for 2 to 3 weeks on an auxin-containing medium from which they are transferred to soil. Hardened shoots are grown under high humidity and short days in a greenhouse.

Approximately 45 independent T0 rice transformants are generated for one construct. The primary transformants are transferred from a tissue culture chamber to a greenhouse. After a quantitative PCR analysis to verify copy number of the T-DNA insert, only single copy transgenic plants that exhibit tolerance to the selection agent are kept for harvest of T1 seed. Seeds are then harvested three to five months after transplanting. The method yielded single locus transformants at a rate of over 50% (Aldemita and Hodges1996, Chan et al. 1993, Hiei et al. 1994).

### Corn Transformation

Transformation of maize (Zea mays) is performed with a modification of the method described by Ishida et al. (1996) Nature Biotech 14(6): 745-50. Transformation is genotype-dependent in corn and only specific genotypes are amenable to transformation and regeneration. The inbred line A188 (University of Minnesota) or hybrids with A188 as a parent are good sources of donor material for transformation, but other genotypes can be used successfully as well. Ears are harvested from corn plant approximately 11 days after pollination (DAP) when the length of the immature embryo is about 1 to 1.2 mm. Immature embryos are cocultivated with Agrobacterium tumefaciens containing the expression vector, and transgenic plants are recovered through organogenesis. Excised embryos are grown on callus induction medium, then maize regeneration medium, containing the selection agent (for example imidazolinone but various selection markers can be used). The Petri plates are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Wheat Transformation

Transformation of wheat is performed with the method described by Ishida et al. (1996) Nature Biotech 14(6): 745-50. The cultivar Bobwhite (available from CIMMYT, Mexico) is commonly used in transformation. Immature embryos are co-cultivated with Agrobacterium tumefaciens containing the expression vector, and transgenic plants are recovered through organogenesis. After incubation with Agrobacterium, the embryos are grown in vitro on callus induction medium, then regeneration medium, containing the selection agent (for example imidazolinone but various selection markers can be used). The Petri plates are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Soybean Transformation

Soybean is transformed according to a modification of the method described in the Texas A&M U.S. Pat. No. 5,164,310. Several commercial soybean varieties are amenable to transformation by this method. The cultivar Jack (available from the Illinois Seed foundation) is commonly used for transformation. Soybean seeds are sterilised for in vitro sowing. The hypocotyl, the radicle and one cotyledon are excised from seven-day old young seedlings. The epicotyl and the remaining cotyledon are further grown to develop axillary nodes. These axillary nodes are excised and incubated with Agrobacterium tumefaciens containing the expression vector. After the cocultivation treatment, the explants are washed and transferred to selection media. Regenerated shoots are excised and placed on a shoot elongation medium. Shoots no longer than 1 cm are placed on rooting medium until roots develop. The rooted shoots are transplanted to soil in the greenhouse. T1 seeds are produced from plants that exhibit tolerance to the selection agent and that contain a single copy of the T-DNA insert.

### Rapeseed/Canola Transformation

Cotyledonary petioles and hypocotyls of 5-6 day old young seedling are used as explants for tissue culture and transformed according to Babic et al. (1998, Plant Cell Rep 17: 183-188). The commercial cultivar Westar (Agriculture Canada) is the standard variety used for transformation, but other varieties can also be used. Canola seeds are surface-sterilized for in vitro sowing. The cotyledon petiole explants with the cotyledon attached are excised from the in vitro seedlings, and inoculated with Agrobacterium (containing the expression vector) by dipping the cut end of the petiole explant into the bacterial suspension. The explants are then cultured for 2 days on MSBAP-3 medium containing 3 mg/l BAP, 3% sucrose, 0.7 Phytagar at 23° C., 16 hr light. After two days of co-cultivation with Agrobacterium, the petiole explants are transferred to MSBAP-3 medium containing 3 mg/l BAP, cefotaxime, carbenicillin, or timentin (300 mg/l) for 7 days, and then cultured on MSBAP-3 medium with cefotaxime, carbenicillin, or timentin and selection agent until shoot regeneration. When the shoots are 5-10 mm in length, they are cut and transferred to shoot elongation medium (MSBAP-0.5, containing 0.5 mg/l BAP). Shoots of about 2 cm in length are transferred to the rooting medium (MS0) for root induction. The rooted shoots are transplanted to soil in the greenhouse. T1 seeds are produced from plants that exhibit tolerance to the selection agent and that contain a single copy of the T-DNA insert.

### Alfalfa Transformation

A regenerating clone of alfalfa (Medicago sativa) is transformed using the method of (McKersie et al., 1999 Plant Physiol 119: 839-847). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown DCW and A Atanassov (1985. Plant Cell Tissue Organ Culture 4: 111-112). Alternatively, the RA3 variety (University of Wisconsin) has been selected for use in tissue culture (Walker et al., 1978 Am J Bot 65:654-659). Petiole explants are cocultivated with an overnight culture of Agrobacterium tumefaciens C58C1 pMP90 (McKersie et al., 1999 Plant Physiol 119: 839-847) or LBA4404 containing the expression vector. The explants are cocultivated for 3 d in the dark on SH induction medium containing 288 mg/L Pro, 53 mg/L thioproline, 4.35 g/L K2SO4, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings were transplanted into pots and grown in a greenhouse. T1 seeds are produced from plants that exhibit tolerance to the selection agent and that contain a single copy of the T-DNA insert.

### Cotton Transformation

Cotton is transformed using Agrobacterium tumefaciens according to the method described in U.S. Pat. No. 5,159,135. Cotton seeds are surface sterilised in 3% sodium hypochlorite solution during 20 minutes and washed in distilled water with 500 μg/ml cefotaxime. The seeds are then transferred to SH-medium with 50 μg/ml benomyl for germination. Hypocotyls of 4 to 6 days old seedlings are removed, cut into 0.5 cm pieces and are placed on 0.8% agar. An Agrobacterium suspension (approx. 108 cells per ml, diluted from an overnight culture transformed with the gene of interest and suitable selection markers) is used for inoculation of the hypocotyl explants. After 3 days at room temperature and lighting, the tissues are transferred to a solid medium (1.6 g/l Gelrite) with Murashige and Skoog salts with B5 vitamins (Gamborg et al., Exp. Cell Res. 50:151-158 (1968)), 0.1 mg/l 2,4-D, 0.1 mg/l 6-furfurylaminopurine and 750 μg/ml MgCL2, and with 50 to 100 μg/ml cefotaxime and 400-500 μg/ml carbenicillin to kill residual bacteria. Individual cell lines are isolated after two to three months (with subcultures every four to six weeks) and are further cultivated on selective medium for tissue amplification (30° C., 16 hr photoperiod). Transformed tissues are subsequently further cultivated on non-selective medium during 2 to 3 months to give rise to somatic embryos. Healthy looking embryos of at least 4 mm length are transferred to tubes with SH medium in fine vermiculite, supplemented with 0.1 mg/l indole acetic acid, 6 furfurylaminopurine and gibberellic acid. The embryos are cultivated at 30° C. with a photoperiod of 16 hrs, and plantlets at the 2 to 3 leaf stage are transferred to pots with vermiculite and nutrients. The plants are hardened and subsequently moved to the greenhouse for further cultivation.

### Sugarbeet Transformation

Seeds of sugarbeet (Beta vulgaris L.) are sterilized in 70% ethanol for one minute followed by 20 min. shaking in 20% Hypochlorite bleach e.g. Clorox® regular bleach (commercially available from Clorox, 1221 Broadway, Oakland, Calif. 94612, USA). Seeds are rinsed with sterile water and air dried followed by plating onto germinating medium (Murashige and Skoog (MS) based medium (see Murashige, T., and Skoog, 1962. A revised medium for rapid growth and bioassays with tobacco tissue cultures. Physiol. Plant, vol. 15, 473-497) including B5 vitamins (Gamborg et al.; Nutrient requirements of suspension cultures of soybean root cells. Exp. Cell Res., vol. 50, 151-8.) supplemented with 10 g/l sucrose and 0.8% agar). Hypocotyl tissue is used essentially for the initiation of shoot cultures according to Hussey and Hepher (Hussey, G., and Hepher, A., 1978. Clonal propagation of sugarbeet plants and the formation of polylpoids by tissue culture. Annals of Botany, 42, 477-9) and are maintained on MS based medium supplemented with 30 g/l sucrose plus 0.25 mg/l benzylamino purine and 0.75% agar, pH 5.8 at 23-25° C. with a 16-hour photoperiod.

Agrobacterium tumefaciens strain carrying a binary plasmid harbouring a selectable marker gene for example nptII is used in transformation experiments. One day before transformation, a liquid LB culture including antibiotics is grown on a shaker (28° C., 150 rpm) until an optical density (O.D.) at 600 nm of ˜1 is reached. Overnight-grown bacterial cultures are centrifuged and resuspended in inoculation medium (O.D. ˜1) including Acetosyringone, pH 5.5.

Shoot base tissue is cut into slices (1.0 cm×1.0 cm×2.0 mm approximately). Tissue is immersed for 30 s in liquid bacterial inoculation medium. Excess liquid is removed by filter paper blotting. Co-cultivation occurred for 24-72 hours on MS based medium incl. 30 g/l sucrose followed by a non-selective period including MS based medium, 30 g/l sucrose with 1 mg/l BAP to induce shoot development and cefotaxim for eliminating the Agrobacterium. After 3-10 days explants are transferred to similar selective medium harbouring for example kanamycin or G418 (50-100 mg/l genotype dependent).

Tissues are transferred to fresh medium every 2-3 weeks to maintain selection pressure. The very rapid initiation of shoots (after 3-4 days) indicates regeneration of existing meristems rather than organogenesis of newly developed transgenic meristems. Small shoots are transferred after several rounds of subculture to root induction medium containing 5 mg/l NAA and kanamycin or G418. Additional steps are taken to reduce the potential of generating transformed plants that are chimeric (partially transgenic). Tissue samples from regenerated shoots are used for DNA analysis.

Other transformation methods for sugarbeet are known in the art, for example those by Linsey & Gallois (Linsey, K., and Gallois, P., 1990. Transformation of sugarbeet (Beta vulgaris) by Agrobacterium tumefaciens. Journal of Experimental Botany; vol. 41, No. 226; 529-36) or the methods published in the international application published as WO9623891 A.

### Sugarcane Transformation

Spindles are isolated from 6-month-old field grown sugarcane plants (see Arencibia A., at al., 1998. An efficient protocol for sugarcane (Saccharum spp. L.) transformation mediated by Agrobacterium tumefaciens. Transgenic Research, vol. 7, 213-22; Enriquez-Obregon G., et al., 1998. Herbicide-resistant sugarcane (Saccharum officinarum L.) plants by Agrabacterium-mediated transformation. Planta, vol. 206, 20-27). Material is sterilized by immersion in a 20% Hypochlorite bleach e.g. Clorox® regular bleach (commercially available from Clorox, 1221 Broadway, Oakland, Calif. 94612, USA) for 20 minutes. Transverse sections around 0.5 cm are placed on the medium in the top-up direction. Plant material is cultivated for 4 weeks on MS (Murashige, T., and Skoog, 1962. A revised medium for rapid growth and bioassays with tobacco tissue cultures. Physiol. Plant, vol. 15, 473-497) based medium incl. B5 vitamins (Gamborg, 0., et al., 1968. Nutrient requirements of suspension cultures of soybean root cells. Exp. Cell Res., vol. 50, 151-8) supplemented with 20 g/l sucrose, 500 mg/l casein hydrolysate, 0.8% agar and 5 mg/l 2,4-D at 23° C. in the dark. Cultures are transferred after 4 weeks onto identical fresh medium.

Agrobacterium tumefaciens strain carrying a binary plasmid harbouring a selectable marker gene for example hpt is used in transformation experiments. One day before transformation, a liquid LB culture including antibiotics is grown on a shaker (28° C., 150 rpm) until an optical density (O.D.) at 600 nm of ˜0.6 is reached. Overnight-grown bacterial cultures are centrifuged and resuspended in MS based inoculation medium (O.D. ˜0.4) including acetosyringone, pH 5.5.

Sugarcane embryogenic calli pieces (2-4 mm) are isolated based on morphological characteristics as compact structure and yellow colour and dried for 20 min. in the flow hood followed by immersion in a liquid bacterial inoculation medium for 10-20 minutes. Excess liquid is removed by filter paper blotting. Co-cultivation occurred for 3-5 days in the dark on filter paper which is placed on top of MS based medium incl. B5 vitamins containing 1 mg/l 2,4-D. After co-cultivation calli are ished with sterile water followed by a non-selective period on similar medium containing 500 mg/l cefotaxime for eliminating the Agrobacterium. After 3-10 days explants are transferred to MS based selective medium incl. B5 vitamins containing 1 mg/l 2,4-D for another 3 weeks harbouring 25 mg/l of hygromycin (genotype dependent). All treatments are made at 23° C. under dark conditions.

Resistant calli are further cultivated on medium lacking 2,4-D including 1 mg/l BA and 25 mg/l hygromycin under 16 h light photoperiod resulting in the development of shoot structures. Shoots are isolated and cultivated on selective rooting medium (MS based including, 20 g/l sucrose, 20 mg/l hygromycin and 500 mg/l cefotaxime). Tissue samples from regenerated shoots are used for DNA analysis.

Other transformation methods for sugarcane are known in the art, for example from the international application published as WO2010/151634A and the granted European patent EP1831378.

### Example 11

### Cloning of the sequences as shown in Table I, column 5 or 7 in Escherichia coli

The inventive sequences as shown in the respective line in Table I, column 5 or 7 are cloned into the plasmids pBR322 (Sutcliffe J. G., Proc. Natl. Acad. Sci. USA, 75, 3737 (1979)), pA-CYC177 (Change and Cohen, J. Bacteriol. 134, 1141 (1978)), plasmids of the pBS series (pBSSK+, pBSSK− and others; Stratagene, LaJolla, USA) or cosmids such as SuperCosi (Stratagene, LaJolla, USA) or Lorist6 (Gibson T. J., Rosenthal A. and Waterson R. H., Gene 53, 283 (1987) for expression in E. coli using known, well-established procedures (see, for example, J. Sambrook et al. “Molecular Cloning: A Laboratory Manual”. Cold Spring Harbor Laboratory Press (1989) or F. M. Ausubel et al., “Current Protocols in Molecular Biology”, John Wiley & Sons (1994)).

### Example 12

### Determining the Expression of the Mutant/Transgenic Protein in a Host Cell or Plant

A suitable method for determining the transcription quantity of the mutant, or transgenic, gene (a sign for the amount of mRNA which is available for the translation of the gene product) is to carry out a Northern blot (see, for example, Ausubel et al., “Current Protocols in Molecular Biology”, Wiley, New York (1988)), where a primer which is designed in such a way that it binds to the gene of interest is provided with a detectable marker (usually a radioactive or chemiluminescent marker) so that, when the total RNA of a culture of the organism is extracted, separated on a gel, applied to a stable matrix and incubated with this probe, the binding and quantity of the binding of the probe indicates the presence and also the amount of mRNA for this gene. Another method is a quantitative PCR. This information detects the extent to which the gene has been transcribed. Total cell RNA can be isolated for example from yeasts or E. coli by a variety of methods, which are known in the art, for example with the Ambion kit according to the instructions of the manufacturer or as described in Edgington et al., Promega Notes Magazine Number 41, 14 (1993).

Standard techniques, such as Western blot, may be employed to determine the presence or relative amount of protein translated from this mRNA (see, for example, Ausubel et al. “Current Protocols in Molecular Biology”, Wiley, New York (1988)). In this method, total cell proteins are extracted, separated by gel electrophoresis, transferred to a matrix such as nitrocellulose and incubated with a probe, such as an antibody, which binds specifically to the desired protein. This probe is usually provided directly or indirectly with a chemiluminescent or colorimetric marker, which can be detected readily. The presence and the observed amount of marker indicate the presence and the amount of the sought mutant protein in the cell. However, other methods are also known.

### Example 13

### Plant Culture (Arabidopsis) for Bioanalytical Analyses

For the bioanalytical analyses of the transgenic plants, the latter were grown uniformly a specific culture facility. To this end the GS-90 substrate as the compost mixture was introduced into the potting machine (Laible System GmbH, Singen, Germany) and filled into the pots. Thereafter, 35 pots were combined in one dish and treated with Previcur. For the treatment, 25 ml of Previcur were taken up in 101 of tap water. This amount was sufficient for the treatment of approximately 200 pots. The pots were placed into the Previcur solution and additionally irrigated over-head with tap water without Previcur. They were used within four days.

For the sowing, the seeds, which had been stored in the refrigerator (at −20° C.), were removed from the Eppendorf tubes with the aid of a toothpick and transferred into the pots with the compost. In total, approximately 5 to 12 seeds were distributed in the middle of the pot.

After the seeds had been sown, the dishes with the pots were covered with matching plastic hood and placed into the stratification chamber for 4 days in the dark at 4° C. The humidity was approximately 90%. After the stratification, the test plants were grown for 22 to 23 days at a 16-h-light, 8-h-dark rhythm at 20° C., an atmospheric humidity of 60% and a CO2 concentration of approximately 400 ppm. The light sources used were Powerstar HQI-T 250 W/D Daylight lamps from Osram, which generate a light resembling the solar color spectrum with a light intensity of approximately 220 E/m2/s-1.

Selection of transgenic plants was depending on the use resistance marker. In case of the bar gene as the resistance marker plantlets were sprayed three times at days 8-10 after sowing with 0.02% BASTA®, Bayer CropScience, Germany, Leverkusen. The resistance plants were thinned when they had reached the age of 14 days. The plants, which had grown best in the center of the pot were considered the target plants. All the remaining plants were removed care-fully with the aid of metal tweezers and discarded.

During their growth, the plants received overhead irrigation with distilled water (onto the compost) and bottom irrigation into the placement grooves. Once the grown plants had reached the age of 23 days, they were harvested. In case their seeds are desired these had been harvested 10 to 12 weeks after sowing (once they are ripe).

### Example 14

### Metabolic Analysis of Transformed Plants

The modifications identified in accordance with the invention, in the content of above-described metabolites, were identified by the following procedure.

### a) Sampling and Storage of the Samples

Sampling was performed directly in the controlled-environment chamber. The plants, or respective parts thereof, like leafs, were cut using small laboratory scissors, rapidly weighed on laboratory scales, transferred into a pre-cooled extraction thimble and placed into an aluminum rack cooled by liquid nitrogen. If required, the extraction thimbles can be stored in the freezer at 80° C. The time elapsing between cutting the plant/plant parts to freezing it in liquid nitrogen amounted to not more than 10 to 20 seconds.

### b) Lyophilization

During the experiment, care was taken that the plants either remained in the deep-frozen state (temperatures<−40° C.) or were freed from water by lyophilization until the first contact with solvents.

The aluminum rack with the plant samples in the extraction thimbles was placed into the pre-cooled (−40° C.) lyophilization facility. The initial temperature during the main drying phase was −35° C. and the pressure was 0.120 mbar. During the drying phase, the parameters were altered following a pressure and temperature program. The final temperature after 12 hours was +30° C. and the final pressure was 0.001 to 0.004 mbar.

After the vacuum pump and the refrigerating machine had been switched off, the system was flushed with air (dried via a drying tube) or argon.

### c) Extraction

### Extraction of Arabidopsis Green Tissue:

Immediately after the lyophilization apparatus had been flushed, the extraction thimbles with the lyophilized plant material were transferred into the 5 ml extraction cartridges of the ASE device (Accelerated Solvent Extractor ASE 200 with Solvent Controller and AutoASE software (DIONEX)).

The 24 sample positions of an ASE device (Accelerated Solvent Extractor ASE 200 with Solvent Controller and AutoASE software (DIONEX)) were filled with plant samples, including some samples for testing quality control.

The polar substances were extracted with approximately 10 ml of methanol/water (80/20, v/v) at T=70° C. and p=140 bar, 5 minutes heating-up phase, 1 minute static extraction. The more lipophilic substances were extracted with approximately 10 ml of methanol/dichloromethane (40/60, v/v) at T=70° C. and p=140 bar, 5 minute heating-up phase, 1 minute static extraction. The two solvent mixtures were extracted into the same glass tubes (centrifuge tubes, 50 ml, equipped with screw cap and pierceable septum for the ASE (DIONEX)).

The solution was treated with commercial available internal standards, such as ribitol, L-glycine-2,2-d2, L alanine-2,3,3,3-d4, methionine-d3, Arginine_(13C), Tryptophan-d5, and α-methylglucopyranoside and methyl nonadecanoate, methyl undecanoate, methyl tridecanoate, methyl pentadecanoate, methyl nonacosanoate

The total extract was treated with 8 ml of water. The solid residue of the plant sample and the extraction thimbles were discarded.

The extract was shaken and then centrifuged for 5 to 10 minutes at at least 1400 g in order to accelerate phase separation. 1 ml of the supernatant methanol/water phase (“polar phase”, col-orless) was removed for the further GC analysis, and 1 ml was removed for the LC analysis. The remainder of the methanol/water phase was discarded. 0.5 ml of the organic phase (“lipid phase”, dark green) was removed for the further GC analysis and 0.5 ml was removed for the LC analysis. All the portions removed were evaporated to dryness using the IR Dancer infrared vacuum evaporator (Hettich). The maximum temperature during the evaporation process did not exceed 40° C. Pressure in the apparatus was not less than 10 mbar.

### Extraction of Arabidopsis Seeds:

3 mg of Arabidopsis seeds are transferred into a 1.2-mL-stainless steel grinding jar and ground and extracted with a mixture of 770 μL methanol and 290 μL water. A solution containing commercially available standard substances (ribitol, L-glycine-2,2-d2, L alanine-2,3,3,3-d4, methionine-methyl-d3, tryptophane-d5, Arginine 13C615N4, Pep3 (Boc-Ala-Gly-Gly-Gly-OH) and α-methylglucopyranoside) is added as internal standard. The extraction is performed using a stainless steel ball and a ball mill (Retsch MM 200, Retsch, Germany) operated at 30 Hz for 3 minutes. After centrifugation at 6000 rpm for 5 min 800 μL of the extraction solvent is transferred into a 2-mL-reaction tube (Eppendorf).

A solution of commercially available internal standard substances (Coenzyme Q1, Coenzyme Q2, Coenzyme Q4, and methyl nonadecanoate, undecanoic acid, tridecanoic acid, penta-decanoic acid, methyl nonacosanoate) is added as internal standard. For the extraction of lipophilic metabolites, 640 μL methylene chloride and 170 μL methanol are added and the sample is extracted in a ball mill operated at 30 Hz for 3 minutes. After centrifugation at 6000 rpm for 5 min 800 μL of the extraction solvent is transferred and combined with the extract of the first extraction step. After the addition of 400 μL of water and a centrifugation step to ensure proper separation of the organic and aqueous layer, two aliquots of 500 μL of the aqueous top layer (polar phase) are taken for GC and LC analysis, respectively.

Two aliquots of 100 μL of the organic bottom layer (lipid phase) are take for GC and LC analysis, respectively.

All the portions removed were evaporated to dryness using the IR Dancer infrared vacuum evaporator (Hettich). The maximum temperature during the evaporation process did not exceed 40° C. Pressure in the apparatus was not less than 10 mbar.

### Extraction of Rice or Corn Seed Material:

20 rice or corn kernels are homogenized with a 50-mL-stainless steel grinding jar and ground with a stainless steel grinding ball using a ball mill (Retsch MM 200, Retsch, Germany) operated at 30 Hz for 3 minutes. The ground samples are lyophilized over night The initial temperature during the main drying phase was −35° C. and the pressure was 0.120 mbar. During the drying phase, the parameters were altered following a pressure and temperature program. The final temperature after 12 hours was +30° C. and the final pressure was 0.001 to 0.004 mbar. After the vacuum pump and the refrigerating machine had been switched off, the system was flushed with air (dried via a drying tube) or argon.
50 mg of the lyophilized kernel material are weighed into glass fibre extraction thimbles and extracted and further processed as described for the Extraction of Arabidopsis green tissue.

### d) Processing the Lipid and Polar Phase for the LC/MS or LC/MS/MS Analysis

The lipid extract, which had been evaporated to dryness was taken up in mobile phase. The polar extract, which had been evaporated to dryness was taken up in mobile phase.

### LC-MS Analysis:

The LC part was carried out on a commercially available LCMS system from Agilent Technologies, USA. For polar extracts 10 μl are injected into the system at a flow rate of 200 μl/min. The separation column (Reversed Phase C18) was maintained at 15° C. during chromatography. For lipid extracts 5 μl are injected into the system at a flow rate of 200 μl/min. The separation column (Reversed Phase C18) was maintained at 30° C. HPLC was performed with gradient elution.

The mass spectrometric analysis was performed on an Applied Biosystems API 4000 triple quadrupole instrument with turbo ion spray source. For polar extracts the instrument measured in negative ion mode in MRM-mode and fullscan mode from 100-1000 amu. For lipid extracts the instrument measured in positive ion mode in MRM-mode fullscan mode from 100-1000 amu. MS analysis is described in more detail in patent publication number WO 03/073464 (Walk and Dostler).

### e) Derivatization of the Lipid and Polar Phase for the GC/MS Analysis

### Derivatization of the Lipid Phase for the GC/MS Analysis:

For the transmethanolysis, a mixture of 140 μl of chloroform, 37 μl of hydrochloric acid (37% by weight HCl in water), 320 μl of methanol and 20 μl of toluene was added to the evaporated ex-tract. The vessel was sealed tightly and heated for 2 hours at 100° C., with shaking. The solution was subsequently evaporated to dryness. The residue was dried completely.

The methoximation of the carbonyl groups was carried out by reaction with methoxyamine hy-drochloride (5 mg/ml in pyridine, 100 μl for 1.5 hours at 60° C.) in a tightly sealed vessel. 20 μl of a solution of odd-numbered, straight-chain fatty acids (solution of each 0.3 mg/mL of fatty acids from 7 to 25 carbon atoms and each 0.6 mg/mL of fatty acids with 27, 29 and 31 carbon atoms in 3/7 (v/v) pyridine/toluene) were added as time standards. Finally, the derivatization with 100 μl of N methyl-N-(trimethylsilyl)-2,2,2-trifluoroacetamide (MSTFA) was carried out for 30 minutes at 60° C., again in the tightly sealed vessel. The final volume before injection into the GC was 220 μl.

### Derivatization of the Polar Phase for the GC/MS Analysis:

The methoximation of the carbonyl groups was carried out by reaction with methoxyamine hydrochloride (5 mg/ml in pyridine, 50 μl for 1.5 hours at 60° C.) in a tightly sealed vessel. 10 μl of a solution of odd-numbered, straight-chain fatty acids (solution of each 0.3 mg/mL of fatty acids from 7 to 25 carbon atoms and each 0.6 mg/mL of fatty acids with 27, 29 and 31 carbon atoms in 3/7 (v/v) pyridine/toluene) were added as time standards. Finally, the derivatization with 50 μl of N methyl-N-(trimethylsilyl)-2,2,2-trifluoroacetamide (MSTFA) was carried out for 30 minutes at 60° C., again in the tightly sealed vessel. The final volume before injection into the GC was 110 μl.

### f) GC-MS Analysis

The GC-MS systems consisted of an Agilent 6890 GC coupled to an Agilent 5973 MSD. The autosamplers were CompiPal or GCPaI from CTC. For the analysis usual commercial capillary separation columns (30 m×0.25 mm×0.25 μm) with different poly-methyl-siloxane stationary phases containing 0% up to 35% of aromatic moieties, depending on the analysed sample materials and fractions from the phase separation step, were used (for example: DB-1 ms, HP-5 ms, DB-XLB, DB-35 ms, Agilent Technologies). Up to 1 μL of the final volume was injected splitless and the oven temperature program was started at 70° C. and ended at 340° C. with different heating rates depending on the sample material and fraction from the phase separation step in order to achieve a sufficient chromatographic separation and number of scans within each analyte peak. Usual GC-MS standard conditions, for example constant flow with nominal 1 to 1.7 ml/min. and helium as the mobile phase gas were used. Ionisation was done by electron impact with 70 eV, scanning within a m/z range from 15 to 600 with scan rates from 2.5 to 3 scans/sec and standard tune conditions.

### g) Analysis of the Various Plant Samples

The samples were measured in individual series of 20 to 21 plant or seed samples each (also referred to as sequences), each sequence containing at least 5 wild-type plants or seed samples as controls. Seed samples were from individual plants. The peak area of each analyte was divided by the peak area of the respective internal standard. The data were standardized for the fresh weight established for the plant or seed sample, respectively. The values calculated thus were related to the wild-type control group by being divided by the mean of the corresponding data of the wild-type control group of the same sequence. The values obtained were referred to as ratio_by_WT, they are comparable between sequences and indicate how much the analyte concentration in the mutant differs in relation to the wild-type control. Appropriate controls were done before to proof that the vector and transformation procedure itself has no significant influence on the metabolic composition of the plants. Therefore the described changes in comparison with wild types were caused by the introduced gene constructs. At least 3-5 independent lines were analyzed in two independent experiments for each construct.

### Example 15

### Fine Chemical Measurements

Purification Of a Fine Chemical Saccharide e.g. R Myo-Inosito Sucrose

Saccharides (carbohydrates) can for example be detected advantageously via traditional methods of sugar analysis coupled to chromatography use a Refractive Index Detector (RID) due to a lack of a UV-absorbing chromophore on sugar molecules. Other detectors, like Mass Spectrometry (MS) or Pulsed Amperometric Detection (PAD), are used also. Methods of sugar analysis are capillary electrophoresis, GC, HPLC or LC.

Saccharides (carbohydrates) are detected by GC or LC combined with MS. Traditional methods of sugar analysis coupled to chromatography use a Refractive Index Detector (RID) due to a lack of a UV-absorbing chromophore on sugar molecules. Other detectors, like Mass Spectrometry (MS) or Pulsed Amperometric Detection (PAD), are used also.

In one embodiment of the invention the fructose can be detected by chromatography, thin layer chromatography, Gaschromatography (GC), liquid-chromatographie (LC), capillary electropho-resis and HPLC. Alternatively fructose can be detected and analized by bio-sensors: a am-perometric enzyme electrode for fructose analysis was constructed, by co-immobilization of a pyrrolo quinoline quinone (PQQ) enzyme (Gluconobacter sp. fructose-5-dehydrogenase, FDH, EC-1.1.99.11) with a mediator in a thin polypyrrole (PP) membrane (Anal. Chim. Acta; (1993) 281, 3, 527-33). Two amperometric biosensors for fructose detection were developed by immobilizing d-fructose 5-dehydrogenase by two different immobilization processes (Analytica Chimica Acta, Volume 374, Number 2, 23 Nov. 1998, pp. 201-208(8)).

The glucose can be detected by Fourier transformed near-infrared (FT-NIR) spectroscopy in diffuse reflectance mode (Liu et al., 2006), by HPLC (siehe z. B. Sánchez-Mata et al., European Food Research and Technology, 2004) or by colourimetric enzyme-assays (Ciantar et al., J Periodontal Res., 2002).

A further method is the analysis of fluorophore-labeled glycans by high-resolution polyacrylat-mide gel electrophoresis (Jackson et al., Anal. Biochem. 216 (1994) 243-52).

The sucrose of the invention is detected in one embodiment by traditional methods of sugar analysis coupled to chromatography use a Refractive Index Detector (RID, Koimur et al., Chromatographia 43, 1996, p. 254-260; Callul et al., J. Chromatogr. 590, 1992, p. 215-222) due to a lack of a UV-absorbing chromophore on sugar molecules. Other detectors, like Mass Spectrometry (MS) or Pulsed Amperometric Detection (PAD, Weston et al., Food Chem. 64, 1999, p. 33-37; Sigvardson et al., J. Pharm. Biomed. Anal. 15, 1996, p. 227-231) are also used. In another embodiment the sucrose is detected by enzyme-linked immunosorbant assay (U.S. Pat. No. 5,972,631), or by Fourier Transform Infrared Detection in Miniaturized Total Analysis Systems for Sucrose Analysis (Anal. Chem. 1997, 69, 2877-2881).

Purification of a Fatty Acid Fine Chemical, e.g. Linoleic Acid and Linolenic Acid.

The microorganism can be disrupted by sonication, grinding in a glass mill, liquid nitrogen and grinding, cooking, or via other applicable methods. After disruption centrifugation may follow. The sediment is resuspended in distilled water, heated for 10 minutes at 100° C., cooled on ice and recentrifuged, followed by extraction for one hour at 90° C. in 0.5 M sulfuric acid in methanol with 2% dimethoxypropane, which leads to hydrolyzed oil and lipid compounds, which give transmethylated lipids. These fatty acid methyl esters are extracted with petroleum ether and the solvent is evaporated lateron. (Analysis of the so obtained fatty acid ester(s) will be performed by GC analysis using a capillary column (Chrompack, WCOT Fused Silica, CP-Wax-52 CB, 25 micrometer, 0.32 mm) at a temperature gradient of between 170° C. and 240° C. for 20 minutes and 5 minutes at 240° C. The identity of the resulting fatty acid methyl esters can be determined using standards which are available from commercial sources (i.e. Sigma).)

TABLE R1

| SeqID | Target | Sequence | Metabolite | Source | Promotor | Method | Min | Max |
|---|---|---|---|---|---|---|---|---|
| 1 | non-targ | Ynl064c | myo- | ARA_LEAF | Big35S | GC | 28 | 50 |
| | | | inositol | | | | | |
| 1 | non-targ | Ynl064c | sucrose | ARA_LEAF | Big35S | GC | 25 | 31 |
| 1 | non- | Ynl064c | linoleic | ARA_LEAF | Big35S | GC | 15 | 25 |
| | targeted | | acid | | | | | |
| 1 | non- | Ynl064c | linolenic | ARA_LEAF | Big35S | GC | 13 | 24 |
| | targeted | | acid | | | | | |

Column 1 shows the SEQ ID NO, Column 2 shows the expression type (targeted or non-targeted), Column 3 shows the “gene name” (sequence), Column 4 shows the metabolite analyzed, Column 5 indicates the A. thaliana source tissue analyzed, Column 6 indicates the used promoter for expression, Column 7 indicates the analytical method. Columns 8 and 9 show the minimum and the maximum increase of the analyzed metabolite (in percent) in comparison to the wild type (ratio_by_WT, given as percent increase).

The term “non-tarp” in Column 2 which shows the expression type means “non-targeted”, i.e. the sequence of SEQ ID NO: 1 was not linked to a plastid, secretory or mitochondrial targeting sequence, or any targeting signal.

### Example 16

### Stress Phenotypic Evaluation Procedure

### Drought

In the cycling drought assay repetitive stress is applied to Arabidopsis plants without leading to desiccation. In a standard experiment soil is prepared as 1:1 (v/v) mixture of nutrient rich soil (GS90, Tantau, Wansdorf, Germany) and quartz sand. Pots (6 cm diameter) were filled with this mixture and placed into trays. Water was added to the trays to let the soil mixture take up appropriate amount of water for the sowing procedure (day 1) and subsequently T2 generation seeds of transgenic A. thaliana plants and their wild-type controls were sown in pots. Then the filled tray was covered with a transparent lid and transferred into a precooled (4° C.-5° C.) and darkened growth chamber. Stratification was established for a period of 3 days in the dark at 4° C.-5° C. or, alternatively, for 4 days in the dark at 4° C. Germination of seeds and growth was initiated at a growth condition of 20° C., 60% relative humidity, 16 h photoperiod and illumination with fluorescent light at 200 μmol/m2s or, alternatively at 220 μmol/m2s. Covers were removed 7-8 days after sowing. BASTA selection was done at day 10 or day 11 (9 or 10 days after sowing) by spraying pots with plantlets from the top. In the standard experiment, a 0.07% (v/v) solution of BASTA concentrate (183 g/l glufosinate-ammonium) in tap water was sprayed once or, alternatively, a 0.02% (v/v) solution of BASTA was sprayed three times. The wild-type control plants were sprayed with tap water only (instead of spraying with BASTA dissolved in tap water) but were otherwise treated identically. Plants were individualized 13-14 days after sowing by removing the surplus of seedlings and leaving one seedling in soil. Transgenic events and wild-type control plants were evenly distributed over the chamber.

The water supply throughout the experiment was limited and plants were subjected to cycles of drought and re-watering. Watering was carried out at day 1 (before sowing), day 14 or day 15, day 21 or day 22, and finally, day 27 or day 28. For measuring biomass production, plant fresh weight was determined one day after the final watering (day 28 or day 29) by cutting shoots and weighing them. Besides weighing, phenotypic information was added in case of plants that differ from the wild type control. Plants were in the stage prior to flowering and prior to growth of inflorescence when harvested. Significance values for the statistical significance of the biomass changes were calculated by applying the ‘student's’ t test (parameters: two-sided, unequal variance). In this experiment, cycling drought resistance or tolerance and biomass production was compared to wild-type plants. The results thereof are summarized in table R2

### Nitrogen Use Efficiency Screen

T1 or T2 plants are grown in potting soil under normal conditions except for the nutrient solution. The pots are watered from transplantation to maturation with a specific nutrient solution containing reduced N nitrogen (N) content, usually between 7 to 8 times less. The rest of the cultivation (plant maturation, seed harvest) is the same as for plants not grown under abiotic stress. Growth and yield parameters are recorded as detailed for growth under normal conditions.

### Salt Stress Screen

T1 or T2 plants are grown on a substrate made of coco fibers and particles of baked clay (Argex) (3 to 1 ratio). A normal nutrient solution is used during the first two weeks after transplanting the plantlets in the greenhouse. After the first two weeks, 25 mM of salt (NaCl) is added to the nutrient solution, until the plants are harvested. Growth and yield parameters are recorded as detailed for growth under normal conditions.

### Example 11

### Results of the Stress Phenotypic Evaluation of the Transgenic Plants

Biomass production was measured by weighing plant rosettes. Biomass increase was calculated as ratio of average weight for transgenic plants compared to the average weight of wild-type control plants from the same experiment. The maximum biomass increase ratio seen within the group of the five transgenic events was more than 1.49. The average ratio of aboveground biomass of transgenic versus wildtype control plants is shown in table R2 and was an increase in above ground biomass of more than 22%.

TABLE R2
Table R2: Biomass production of transgenic A. thaliana
developed under cycling drought growth conditions.

| Seq ID | Target | Sequence | Biomass Increase |
|---|---|---|---|
| 1 | Cytoplasmic | Ynl064c | 1.2248 |

### Example 17

### Engineering Arabidopsis Plants with an Increased Production of a Fine Chemical by (Over)Expressing a DnaJ-Like Chaperone Protein of the Sequence of any of the SEQ ID NOs of Table II, Preferably SEQ ID NO: 2 or 42 Using Tissue-Specific and/or Stress Inducible Promoters

Transgenic Arabidopsis plants are created as in example 9 to express the DnaJ-like chaperone gene under the control of a tissue-specific and/or stress inducible promoter.

T2 generation plants are produced and are grown under standard conditions. The fine chemical production is determined after a total time of 29 to 30 days starting with the sowing. The transgenic Arabidopsis plant produces more of one ore more of the fine chemicals listed in table FC then non-transgenic control plants.

1-15. (canceled)

16. A method for increasing content of any one or more fine chemicals listed in table FC in plants compared to control plants and for enhancing yield-related traits in plants under abiotic environmental stress conditions and/or non-stress conditions in plants relative to control plants, comprising increasing expression in a plant of a nucleic acid encoding a POI polypeptide and increasing the content of any one or more fine chemicals listed in table FC in plants compared to control plants and enhancing yield-related traits in plants under abiotic environmental stress conditions and/or non-stress conditions in plants relative to control plants, wherein said POI polypeptide is a DnaJ like chaperone.

17. A method for enhancing yield-related traits in plants under abiotic environmental stress conditions relative to control plants, comprising increasing expression in a plant of a nucleic acid encoding a POI polypeptide and enhancing yield-related traits in plants under abiotic environmental stress conditions relative to control plants, wherein said POI polypeptide is a DnaJ like chaperone.

18. A method for increasing content of any one or more fine chemicals listed in table FC in plants relative to control plants, comprising increasing expression in a plant of a nucleic acid encoding a POI polypeptide and increasing content of any one or more fine chemicals listed in table FC in plants relative to control plants, wherein said POI polypeptide is a DnaJ like chaperone.

19. The method of claim 16, wherein said increased expression is effected by introducing and expressing in a plant said nucleic acid encoding a POI polypeptide.

20. The method of claim 16, wherein the nucleic acid encoding a DnaJ like chaperone is selected from the group consisting of:
(i) a nucleic acid represented by SEQ ID NO: 1, 3, 5, 7, 9, 11, 13, 15, 17, 19, 21, 23, 25, 27, 29, 31, 33, 35, 37, 39 or 41;
(ii) the complement of a nucleic acid represented by SEQ ID NO: 1, 3, 5, 7, 9, 11, 13, 15, 17, 19, 21, 23, 25, 27, 29, 31, 33, 35, 37, 39 or 41;
(iii) a nucleic acid encoding a POI polypeptide having in increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence represented by SEQ ID NO: 2, 4, 6, 8, 10, 12, 14, 16, 18, 20, 22, 24, 26, 28, 30, 32, 34, 36, 38, 40 or 42 and additionally comprising one or more domains having in increasing order of preference at least 50%, 55%, 60%, 65%, 70%, 75%, 80%, 85%, 90%, 95%, 96%, 97%, 98%, 99% or 100% sequence identity to any one or more of the PFAM domains PF00226, PF01556 and PF00684, and preferably to the conserved domain starting with amino acid 6 up to amino acid 67 and/or to the conserved domain starting with amino acid 143 up to amino acid 208 and/or to the conserved domain starting with amino acid 265 up to amino acid 348 in SEQ ID NO: 2, and further preferably conferring enhanced yield-related traits relative to control plants under abiotic environmental stress conditions and/or non-stress conditions, and/or increased fine chemical content of one or more fine chemicals as listed in table FC;
(iv) a nucleic acid encoding the polypeptide as represented by SEQ ID NO: 2, 4, 6, 8, 10, 12, 14, 16, 18, 20, 22, 24, 26, 28, 30, 32, 34, 36, 38, 40 or 42 preferably as a result of the degeneracy of the genetic code, said nucleic acid can be derived from a polypeptide sequence as represented by SEQ ID NO: 2, 4, 6, 8, 10, 12, 14, 16, 18, 20, 22, 24, 26, 28, 30, 32, 34, 36, 38, 40 or 42, and further preferably conferring enhanced yield-related traits relative to control plants under abiotic environmental stress conditions and/or non-stress conditions, and/or increased fine chemical content of one or more fine chemicals as listed in table FC;
(v) a nucleic acid encoding a POI polypeptide comprising one or more, preferably all three of the consensus patterns of SEQ ID NO: 45, 46 and 47, and further preferably conferring enhanced yield-related traits relative to control plants under abiotic environmental stress conditions and/or non-stress conditions, and/or increased fine chemical content of one or more fine chemicals as listed in table FC; and
(vi) a nucleic acid which hybridizes with the nucleic acid of (ii) under high stringency hybridization conditions and preferably confers enhanced yield-related traits relative to control plants under abiotic environmental stress conditions and/or non-stress conditions, and/or increased fine chemical content of one or more fine chemicals as listed in table FC.

21. The method of claim 16, wherein said enhanced yield-related traits comprise increased biomass and/or increased seed yield relative to control plants.

22. The method of claim 16, wherein said enhanced yield-related traits are obtained under conditions of drought, salt stress or nitrogen deficiency, preferably drought.

23. The method of claim 16, wherein said increased content of one or more fine chemicals is obtained under non-stress conditions.

24. The method of claim 16, wherein said POI polypeptide comprises:
(i) one or more, preferably two, and more preferably all three of the following PFAM domains PF00226, PF01556 and PF00684, and at least one, preferably any two, more preferably all three of the consensus patterns of SEQ ID NO:45, 46 and 47; and/or
(ii) conserved domain starting with amino acid 6 up to amino acid 67 and/or a conserved domain starting with amino acid 143 up to amino acid 208 and/or a conserved domain starting with amino acid 265 up to amino acid 348 in SEQ ID NO: 2.

25. A plant expression construct comprising:
(a) the nucleic acid encoding a DnaJ-like chaperone as defined in claim 20;
(b) one or more control sequences capable of driving expression of the nucleic acid of (a), wherein at least one control sequence is a constitutive promoter operably linked to the nucleic acid of (a); and optionally
(c) a transcription termination sequence.

26. An expression cassette comprising the nucleic acid as defined in claim 20 and operably linked to a non-native, constitutive promoter.

27. A method for increasing the content of any one or more fine chemicals listed in table FC in plants relative to control plants and/or increasing yield-related traits of a plant under stress conditions, preferably under abiotic environmental stress conditions, and/or non-stress conditions, preferably under conditions of limited water availability, more preferably under conditions of drought relative to a control plant, comprising utilizing a construct comprising:
(i) a nucleic acid encoding the POI polypeptide as defined in claim 24;
(ii) one or more control sequences capable of driving expression of the nucleic acid of (i); and optionally
(iii) a transcription termination sequence.

28. The method of claim 19, wherein the POI encoding nucleic acid is operably linked to one or more control sequences, wherein one of said control sequences is a constitutive promoter.

29. Harvestable parts of a plant obtainable by the method of claim 16, wherein said harvestable parts comprise a recombinant nucleic acid encoding said POI polypeptide in a plant expression cassette or a plant expression construct, wherein the harvestable parts have an increased content of one or more fine chemicals listed in table FC compared to harvestable parts from control plants, and wherein said harvestable parts are preferably shoot biomass and/or seeds.

30. Harvestable parts of a plant obtainable by the method of claim 16, wherein said harvestable parts comprise a construct or an expression cassette comprising said nucleic acid encoding a POI polypeptide, and wherein said harvestable parts are preferably shoot biomass and/or seeds.

31. Products derived from a plant obtainable by the method of claim 16 and/or from harvestable parts of said plant, wherein the products comprise a construct or an expression cassette comprising said nucleic acid encoding a POI polypeptide.

32. A method for increasing the content of any one or more fine chemicals listed in table FC in plants relative to control plants and/or increasing yield-related traits of a plant under stress conditions, preferably under abiotic environmental stress conditions, and/or non-stress conditions, preferably under conditions of limited water availability, more preferably under conditions of drought relative to a control plant, comprising utilizing the nucleic acid encoding a DnaJ-like chaperone as defined in claim 20.

33. A method for the production of a product with increased content of any one or more fine chemicals listed in table FC relative to a product from a control plant, comprising:
(a) growing a plant obtainable by the method of claim 16; and
(b) producing a product from or by:
(i) said plant; or
(ii) parts, including seeds, of said plant,
wherein said product has increased content of any one or more fine chemicals listed in table FC relative to a product from a control plant.

34. The method of claim 33, wherein the product comprises a recombinant nucleic acid encoding the DnaJ-like chaperone.

35. A plant transformed with the construct of claim 25 or an expression cassette comprising said construct, wherein the plant has increased yield-related traits under abiotic stress conditions and/or increased content of any one or more fine chemicals listed in table FC under abiotic environmental stress conditions and/or non-stress conditions compared to a control plant.

36. An agricultural product comprising the nucleic acid as defined in claim 20, or an expression cassette or a construct comprising said nucleic acid, wherein the agricultural product has an increased content of any one or more fine chemicals listed in table FC compared to an agricultural product produced from a control plant.

37. A recombinant chromosomal DNA comprising the construct of claim 25 or an expression cassette comprising said construct.

38. The construct of claim 25, or an expression cassette comprising said construct, or a recombinant chromosomal DNA comprising said construct or said expression cassette, wherein said construct, said expression cassette or said recombinant chromosome is comprised in a plant cell.

39. The method of claim 16, wherein the plant is selected from the group consisting of maize, wheat, rice, soybean, cotton, oilseed rape including canola, sugarcane, sugar beet and alfalfa.

40. The method of claim 16, wherein the plant is a sugarcane plant with increased biomass and/or increased sucrose content of the stems.

41. A host cell comprising the construct of claim 25 or an expression cassette comprising said construct, wherein the host cell is a microorganism.

42. A process for the production of any one or more fine chemicals listed in table FC, comprising:
(a) increasing or generating the activity of a DnaJ-like chaperone non-targeted in a nonhuman organism or a part thereof, preferably a microorganism, a plant cell, a plant or a part thereof, as compared to a corresponding non-transformed wild type non-human organism or a part thereof;
(b) growing the non-human organism or a part thereof under conditions which permit the production of any one or more fine chemicals listed in table FC or a composition comprising any one or more fine chemicals listed in table FC in said non-human organism or in the culture medium surrounding said non-human organism; and
(c) producing one or more fine chemicals listed in table FC or a composition comprising any one or more fine chemicals listed in table FC.

43. The method of claim 16, wherein the fine chemical is sucrose, myo-inositol, linoleic acid, linolenic acid, or a combination of any of sucrose, myo-inositol, linoleic acid, linolenic acid.
